SEQUENCE LISTING (85 sequences)

SEQ ID NO: 1 - DNA, 4043 bp, Aedes aegypti
  source 1..4043: mol_type = unassigned DNA
tccggcgagctggactgtgaccgggtacgaaaatcggcacatggcgaccgtgacgcagacggagtgcgggtgacggagatgttctcctcgtccggccagtgtctgtgtctgctggaacgcttcggaaagtagatgcaacaaatgtcgtatcctgtttgagggattgaattattggggggcgaaagtggggaaagaaaatgtaaaatagaacaaattatttattgtttatagaaaagtgtaatgctgttaagaggttagtatactcaatgtctttccatcaaatacggttagaaattgttcattctatggagattgcacgacgccacgagtttgattttaccttttgatgtttgtcacaaacagcactaggtactgtaattttgaggtgatgcaaataaattttgaaccatcctgctgaacacaaatttattagacgtatttcgacgtttggtgagctcgtttccatacttgcctatcttacgttacttcaatgaacaactaaatacacatttgtatgcgcttatcccctaaagacgggtgataatgtcaactgtttagctaatttccaaaacaatcaaaccttgatacgagataactgactttgcatctctcaaactacgattaatagaaagctatagcaaattatgtacttagatagcattggaaagatgacgctttccgctacctcataacgccattggctttatctatatgactgttcatagctggagagatggatttgaaggtctaattctaagttatcgcatatcaaggtatcgttgtgctggaaaatctgatctgacagtatcgaaatagcgcaactctttgtacccaataatggaaagttctgatttattgtatttataaaaacgttgtctatttgttccgatttcaggtcgtcaaatcacttgctagtaaataacgtctccatagcaattatcattattatatactaaatgaaacgagctcaccaattagatagtttcaaacagttatacagttgcttcaaacaacatacatacatgccttgataagtaccgtgcgccaaatcgagctcgcaacatgagtatgaaaagccactagtaacacattgataatcagcatagaatttaaaataaaataatattttatactgtggatatcttcataacactgcacgctgatataaaattcagaactacaaaaggatcgttgaaattttacgtaactcaacagagtaagggagggggtcttccgaaatgctacggtctatacacaaaatttaaatttttcacacaaaagccgttacgtggaggagggaggggttctaaaattggcaaactttgcgtcacgtaatatttgaatcatcccaaagaaaataaacttctatgctgattataacgcgcgcagaacaatggttgcggtgagagacattaacaggcttgtttgtggtgctgaaatagaacttgtcatcaatatgttttagctggttaaactatacaatcattacgtagcctgaaaatcacccttgaaaaggccgaatatatgacgaaaagacacactctccaactcaaaaggcaaactcaacgtggtcgtgcacaacctccaatagcagtacctgtcggagccgtttggcaacggccagctaccaaaatacgctcgcaatggcatgcaagctacagagagataagtgtttatcagatcattttgggcaccgaaaccgaccgatgtcggaaacgattgaagagatataatctctggtttgtagattgtaggatggttggttgaagatccggtttcccggattttttcggatggatgttgcttgttgatgattctgctgtcgtcgtttttttccggtggcagatggaacagcctcacttcggctttcgaaacacaatcttcaaagttaagtactactgctgttttgcattttttaaattttccctctgaaacttgctttgcactattggtttcatcacttgtttgcactttcacactctttaggaacgctgtctcacaagtagagctcacgtggtagccccagaatggctgcatgtcgctattatcgttaaacagcatttgcactgtggtcattatgttgtttgtattagagttcgtcgcgttcgtggaattgggaaggagaagatacagaattactcaaatgaaaccattccacgggagaatacatttcaggtttaatcttattcttctaggacgaaagcccatcgacagagtcttgcaggcttccgtcgatcgactttcacccgtggatgctaaggaagcttataatgacctcaacattctccgcgcacaggttggctctattctgtttcacagtttccggtgcagttgtgacgaactcagacgaatacccacgattgtatgtccaacctcattttttatctttgtaaactaacgtcgaaaaatctagatactacatttctgctttgcttcatcttacactaatcactagtttgaacttgcgggttttccgttatgctttgtaaatatgcgatgctttagagttttcttcgttccgattcttctttgcattcgattgcttcttccgtcgaatcgatctgatcttcgtggtttattcttgtttcggttcgacctttgccgcagcgcagtgggtcgtgctgatcgtgtaaaaagtctatcatccggactggcgcgtcgtactgcgcaactctacaccgtcgaacatgttcagattgtgcaatcgtgagtattcattgaccacggcttgacctgcgaggcagagaagaacagttggatttttcggatattggtacgacccgggggccgcgttgtcatcagttgcatgaatcgttggtccaagttcgacgaaacgatatggacatcggtgtttcggtggaccaagatcgacgacacgatcttggtcatgagtgtttttcggtggacctagagatattgcaacgaccggagtggaatacgacggtacgatgttggtttgtactgttctggacgctagttacttcattgatacgataaagtttacattcgtcatatctcttgcttttcttgaatccaaatgcttaggacgtgtaaccttcacaaaccgtcataaatcagtttcgattcactacatgttgtagttattcaggctcttataattgaatattcaaaaatcgaattttctattttatcttgatcagaggatataactcgcttaaaatgcacaattttattagcgacaccatgtggatttgttttaattgaaacctctatcttctcatatgtatcacgatataaaatgctcattttattgactgtttaacgataaacttgcgacgatcgacgcaccaccgacctaattccattgtggaagcaaggggcactgcaataccgaaatgtgaagtaaatttcaaatctgctattatagacgatgatctaatactcttgaatggtcttaaacgtgagttgtatttcaagaagttatgacgattcgattttggggccattatgaccccaaaacccagccaacgtaacttttattagtacagacagaaggtcaagcgtgcaagtctttcatccgtgtgtcaataaggccatcagttgaaaccgtgtcaattaaccctccagttaacccttttaacttttaccaggacaaaccaatgacttcgtgcgcaaattccaccactcgttgtctcaggccttgagttgttgtttgataagaatgggggatgtcaagtcggggagcgtagcccaacaggctacggaaactgcatgatggcagtgtttgatccagggcactgttgggaatagactccgtcgaccgaagatcccagatgtcctgaaactcaataataagcgttagcagttacaaaatgggagcacccaggaagtgagtgacacccgatcgatacctcggaaacagtcccaaccggtaagaacccccataccttcgtcaatccgttggcgcgctttattgacgtctccgtcggcgcctttcagtatcacgtacatcaggggcatcatctcccaggggtatcgcagcttctccaggagccattgagatcgtttgacaagttcgtcgt

SEQ ID NO: 2 - DNA, 225 bp, Drosophila melanogaster
  source 1..225: mol_type = unassigned DNA
cagatcttcgtcaagaccctgaccggcaagaccatcaccctggaggtggagccgagcgataccatcgagaacgtgaaggccaagatccaggacaaggagggcatcccgccggatcagcagcgcctgatcttcgccggacgccagctggaggatggccgcaccctgagcgactacaacatccagaaggagagcaccctgcacctggtgctgcgcctgcgcggtggt

SEQ ID NO: 3 - DNA, 1011 bp, synthetic construct
  misc_feature 1..1011: note = synthetic tetracycline responsive activator
  source 1..1011: mol_type = other DNA
gtcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggg

SEQ ID NO: 4 - DNA, 1011 bp, Dictyosoma burgeri; identical to SEQ ID NO: 3
  source 1..1011: mol_type = unassigned DNA

SEQ ID NO: 5 - DNA, 151 bp, Aedes aegypti
  source 1..151: mol_type = unassigned DNA
gatacgacatttgttgcatctactttccgaagcgttccagcagacacagacactggccggacgaggagaacatctccgtcacccgcactccgtctgcgtcacggtcgccatgtgccgattttcgtacccggtcacagtccagctcgccgga

SEQ ID NO: 6 - DNA, 458 bp, Aedes aegypti
  source 1..458: mol_type = unassigned DNA
ctcaacattctccgcgcacaggttggctctattctgtttcacagtttccggtgcagttgtgacgaactcagacgaatacccacgattgtatgtccaacctcattttttatctttgtaaactaacgtcgaaaaatctagatactacatttctgctttgcttcatcttacactaatcactagtttgaacttgcgggttttccgttatgctttgtaaatatgcgatgctttagagttttcttcgttccgattcttctttgcattcgattgcttcttccgtcgaatcgatctgatcttcgtggtttattcttgtttcggttcgacctttgccgcagcgcagtgggtcgtgctgatcgtgtaaaaagtctatcatccggactggcgcgtcgtactgcgcaactctacaccgtcgaacatgttcagattgtgcaatcgtgagtattcattgaccacggcttgac

SEQ ID NO: 7 - DNA, 469 bp, Aedes aegypti
  source 1..469: mol_type = unassigned DNA
tgcaaatgctgtttaacgataatagcgacatgcagccattctggggctaccacgtgagctctacttgtgagacagcgttcctaaagagtgtgaaagtgcaaacaagtgatgaaaccaatagtgcaaagcaagtttcagagggaaaatttaaaaaatgcaaaacagcagtagtacttaactttgaagattgtgtttcgaaagccgaagtgaggctgttccatctgccaccggaaaaaaacgacgacagcagaatcatcaacaagcaacatccatccgaaaaaatccgggaaaccggatcttcaaccaaccatcctacaatctacaaaccagagattatatctcttcaatcgtttccgacatcggtcggtttcggtgcccaaaatgatctgataaacacttatctctctgtagcttgcatgccattgcgagcgtattttggtagctggccgttgccaaacggctccgacag

SEQ ID NO: 8 - DNA, 886 bp, Aedes aegypti
  source 1..886: mol_type = unassigned DNA
tacttatcaaggcatgtatgtatgttgtttgaagcaactgtataactgtttgaaactatctaattggtgagctcgtttcatttagtatataataatgataattgctatggagacgttatttactagcaagtgatttgacgacctgaaatcggaacaaatagacaacgtttttataaatacaataaatcagaactttccattattgggtacaaagagttgcgctatttcgatactgtcagatcagattttccagcacaacgataccttgatatgcgataacttagaattagaccttcaaatccatctctccagctatgaacagtcatatagataaagccaatggcgttatgaggtagcggaaagcgtcatctttccaatgctatctaagtacataatttgctatagctttctattaatcgtagtttgagagatgcaaagtcagttatctcgtatcaaggtttgattgttttggaaattagctaaacagttgacattatcacccgtctttaggggataagcgcatacaaatgtgtatttagttgttcattgaagtaacgtaagataggcaagtatggaaacgagctcaccaaacgtcgaaatacgtctaataaatttgtgttcagcaggatggttcaaaatttatttgcatcacctcaaaattacagtacctagtgctgtttgtgacaaacatcaaaaggtaaaatcaaactcgtggcgtcgtgcaatctccatagaatgaacaatttctaaccgtatttgatggaaagacattgagtatactaacctcttaacagcattacacttttctataaacaataaataatttgttctattttacattttctttccccactttcgccccccaataattcaatccctcaaacag

SEQ ID NO: 9 - DNA, 558 bp, Aedes aegypti
  source 1..558: mol_type = unassigned DNA
gtactgctattggaggttgtgcacgaccacgttgagtttgccttttgagttggagagtgtgtcttttcgtcatatattcggccttttcaagggtgattttcaggctacgtaatgattgtatagtttaaccagctaaaacatattgatgacaagttctatttcagcaccacaaacaagcctgttaatgtctctcaccgcaaccattgttctgcgcgcgttataatcagcatagaagtttattttctttgggatgattcaaatattacgtgacgcaaagtttgccaattttagaacccctccctcctccacgtaacggcttttgtgtgaaaaatttaaattttgtgtatagaccgtagcatttcggaagaccccctcccttactctgttgagttacgtaaaatttcaacgatccttttgtagttctgaattttatatcagcgtgcagtgttatgaagatatccacagtataaaatattattttattttaaattctatgctgattatcaatgtgttactagtggcttttcatactcatgttgcgagctcgatttggcgcac

SEQ ID NO: 10 - DNA, 209 bp, Aedes aegypti
  source 1..209: mol_type = unassigned DNA
gtcattataagcttccttagcatccacgggtgaaagtcgatcgacggaagcctgcaagactctgtcgatgggctttcgtcctagaagaataagattaaacctgaaatgtattctcccgtggaatggtttcatttgagtaattctgtatcttctccttcccaattccacgaacgcgacgaactctaatacaaacaacataatgaccacag

SEQ ID NO: 11 - DNA, 584 bp, Aedes aegypti
  source 1..584: mol_type = unassigned DNA
ggtcggtggtgcgtcgatcgtcgcaagtttatcgttaaacagtcaataaaatgagcattttatatcgtgatacatatgagaagatagaggtttcaattaaaacaaatccacatggtgtcgctaataaaattgtgcattttaagcgagttatatcctctgatcaagataaaatagaaaattcgatttttgaatattcaattataagagcctgaataactacaacatgtagtgaatcgaaactgatttatgacggtttgtgaaggttacacgtcctaagcatttggattcaagaaaagcaagagatatgacgaatgtaaactttatcgtatcaatgaagtaactagcgtccagaacagtacaaaccaacatcgtaccgtcgtattccactccggtcgttgcaatatctctaggtccaccgaaaaacactcatgaccaagatcgtgtcgtcgatcttggtccaccgaaacaccgatgtccatatcgtttcgtcgaacttggaccaacgattcatgcaactgatgacaacgcggcccccgggtcgtaccaatatccgaaaaatccaactgttcttctctgcctcgcag

SEQ ID NO: 12 - DNA, 591 bp, Aedes aegypti
  source 1..591: mol_type = unassigned DNA
gtatgggggttcttaccggttgggactgtttccgaggtatcgatcgggtgtcactcacttcctgggtgctcccattttgtaactgctaacgcttattattgagtttcaggacatctgggatcttcggtcgacggagtctattcccaacagtgccctggatcaaacactgccatcatgcagtttccgtagcctgttgggctacgctccccgacttgacatcccccattcttatcaaacaacaactcaaggcctgagacaacgagtggtggaatttgcgcacgaagtcattggtttgtcctggtaaaagttaaaagggttaactggagggttaattgacacggtttcaactgatggccttattgacacacggatgaaagacttgcacgcttgaccttctgtctgtactaataaaagttacgttggctgggttttggggtcataatggccccaaaatcgaatcgtcataacttcttgaaatacaactcacgtttaagaccattcaagagtattagatcatcgtctataatagcagatttgaaatttacttcacatttcggtattgcagtgccccttgcttccacaatggaatta

SEQ ID NO: 13 - DNA, 135 bp, Aedes aegypti
  source 1..135: mol_type = unassigned DNA
acgacgaacttgtcaaacgatctcaatggctcctggagaagctgcgatacccctgggagatgatgcccctgatgtacgtgatactgaaaggcgccgacggagacgtcaataaagcgcgccaacggattgacgaag

SEQ ID NO: 14 - DNA, 296 bp, synthetic construct
  misc_feature 1..296: note = Synthetic sequence with 7 TetO sequence motifs from Tn10
  source 1..296: mol_type = other DNA
tcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtc

SEQ ID NO: 15 - DNA, 184 bp, Trichoplusia ni
  source 1..184: mol_type = unassigned DNA
taagaggcgcggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataat

SEQ ID NO: 16 - DNA, 309 bp, Trichoplusia ni
  source 1..309: mol_type = unassigned DNA
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 17 - DNA, 509 bp, Autographa californica multiple nucleopolyhedrovirus
  source 1..509: mol_type = unassigned DNA
gctttacgagtagaattctacgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaac

SEQ ID NO: 18 - DNA, 630 bp, Autographa californica multiple nucleopolyhedrovirus
  source 1..630: mol_type = unassigned DNA
gttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttaccgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaag

SEQ ID NO: 19 - DNA, 63 bp, synthetic construct
  misc_feature 1..63: note = Synthetic sequence
  source 1..63: mol_type = other DNA
gtaagttttcccgttcttttctgggttcttcccttttgctcatccttgctgcactaccttcag

SEQ ID NO: 20 - DNA, 42 bp, synthetic construct
  misc_feature 1..42: note = Synthetic sequence
  source 1..42: mol_type = other DNA
agagtcgatcccaccccacccaagaagaagcgcaaaccggtc

SEQ ID NO: 21 - DNA, 33 bp, synthetic construct
  misc_feature 1..33: note = Synthetic sequence
  source 1..33: mol_type = other DNA
cgacccaagaaaaagcggaaggtggaggacccg

SEQ ID NO: 22 - DNA, 228 bp, Simian virus 40
  source 1..228: mol_type = unassigned DNA
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatc

SEQ ID NO: 23 - DNA, 130 bp, Drosophila melanogaster
  source 1..130: mol_type = unassigned DNA
gcagattgtttagcttgttcagctgcgcttgtttatttgcttagctttcgcttagcgacgtgttcactttgcttgtttgaattgaattgtcgctccgtagacgaagcgcctctatttatactccggcgct

SEQ ID NO: 24 - DNA, 858 bp, Escherichia coli
  source 1..858: mol_type = unassigned DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 25 - DNA, 589 bp, Escherichia coli
  source 1..589: mol_type = unassigned DNA
ttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatgg

SEQ ID NO: 26 - DNA, 12741 bp, synthetic construct
  misc_feature 1..12741: note = Synthetic vector construct
  source 1..12741: mol_type = other DNA
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatctaccgggtgtatcatagcgatgcggccactcgagaaattgaactggctttacgagtagaattctacgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttaccgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagacaattctaattggggtaagttttcccgttcttttctgggttcttcccttttgctcatccttgctgcactaccttcaggtgcaagttgagattcaggccaccatgggtaccgctagagtcgatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggacctaggtaagaggcgcgcctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgacaccaccgcgcaggcgcagcaccaggtgcagggtgctctccttctggatgttgtagtcgctcagggtgcggccatcctccagctggcgtccggcgaagatcaggcgctgctgatccggcgggatgccctccttgtcctggatcttggccttcacgttctcgatggtatcgctcggctccacctccagggtgatggtcttgccggtcagggtcttgacgaagatctgtccggcgagctggactgtgaccgggtacgaaaatcggcacatggcgaccgtgacgcagacggagtgcgggtgacggagatgttctcctcgtccggccagtgtctgtgtctgctggaacgcttcggaaagtagatgcaacaaatgtcgtatcctgtttgagggattgaattattggggggcgaaagtggggaaagaaaatgtaaaatagaacaaattatttattgtttatagaaaagtgtaatgctgttaagaggttagtatactcaatgtctttccatcaaatacggttagaaattgttcattctatggagattgcacgacgccacgagtttgattttaccttttgatgtttgtcacaaacagcactaggtactgtaattttgaggtgatgcaaataaattttgaaccatcctgctgaacacaaatttattagacgtatttcgacgtttggtgagctcgtttccatacttgcctatcttacgttacttcaatgaacaactaaatacacatttgtatgcgcttatcccctaaagacgggtgataatgtcaactgtttagctaatttccaaaacaatcaaaccttgatacgagataactgactttgcatctctcaaactacgattaatagaaagctatagcaaattatgtacttagatagcattggaaagatgacgctttccgctacctcataacgccattggctttatctatatgactgttcatagctggagagatggatttgaaggtctaattctaagttatcgcatatcaaggtatcgttgtgctggaaaatctgatctgacagtatcgaaatagcgcaactctttgtacccaataatggaaagttctgatttattgtatttataaaaacgttgtctatttgttccgatttcaggtcgtcaaatcacttgctagtaaataacgtctccatagcaattatcattattatatactaaatgaaacgagctcaccaattagatagtttcaaacagttatacagttgcttcaaacaacatacatacatgccttgataagtaccgtgcgccaaatcgagctcgcaacatgagtatgaaaagccactagtaacacattgataatcagcatagaatttaaaataaaataatattttatactgtggatatcttcataacactgcacgctgatataaaattcagaactacaaaaggatcgttgaaattttacgtaactcaacagagtaagggagggggtcttccgaaatgctacggtctatacacaaaatttaaatttttcacacaaaagccgttacgtggaggagggaggggttctaaaattggcaaactttgcgtcacgtaatatttgaatcatcccaaagaaaataaacttctatgctgattataacgcgcgcagaacaatggttgcggtgagagacattaacaggcttgtttgtggtgctgaaatagaacttgtcatcaatatgttttagctggttaaactatacaatcattacgtagcctgaaaatcacccttgaaaaggccgaatatatgacgaaaagacacactctccaactcaaaaggcaaactcaacgtggtcgtgcacaacctccaatagcagtacctgtcggagccgtttggcaacggccagctaccaaaatacgctcgcaatggcatgcaagctacagagagataagtgtttatcagatcattttgggcaccgaaaccgaccgatgtcggaaacgattgaagagatataatctctggtttgtagattgtaggatggttggttgaagatccggtttcccggattttttcggatggatgttgcttgttgatgattctgctgtcgtcgtttttttccggtggcagatggaacagcctcacttcggctttcgaaacacaatcttcaaagttaagtactactgctgttttgcattttttaaattttccctctgaaacttgctttgcactattggtttcatcacttgtttgcactttcacactctttaggaacgctgtctcacaagtagagctcacgtggtagccccagaatggctgcatgtcgctattatcgttaaacagcatttgcactgtggtcattatgttgtttgtattagagttcgtcgcgttcgtggaattgggaaggagaagatacagaattactcaaatgaaaccattccacgggagaatacatttcaggtttaatcttattcttctaggacgaaagcccatcgacagagtcttgcaggcttccgtcgatcgactttcacccgtggatgctaaggaagcttataatgacctcaacattctccgcgcacaggttggctctattctgtttcacagtttccggtgcagttgtgacgaactcagacgaatacccacgattgtatgtccaacctcattttttatctttgtaaactaacgtcgaaaaatctagatactacatttctgctttgcttcatcttacactaatcactagtttgaacttgcgggttttccgttatgctttgtaaatatgcgatgctttagagttttcttcgttccgattcttctttgcattcgattgcttcttccgtcgaatcgatctgatcttcgtggtttattcttgtttcggttcgacctttgccgcagcgcagtgggtcgtgctgatcgtgtaaaaagtctatcatccggactggcgcgtcgtactgcgcaactctacaccgtcgaacatgttcagattgtgcaatcgtgagtattcattgaccacggcttgacctgcgaggcagagaagaacagttggatttttcggatattggtacgacccgggggccgcgttgtcatcagttgcatgaatcgttggtccaagttcgacgaaacgatatggacatcggtgtttcggtggaccaagatcgacgacacgatcttggtcatgagtgtttttcggtggacctagagatattgcaacgaccggagtggaatacgacggtacgatgttggtttgtactgttctggacgctagttacttcattgatacgataaagtttacattcgtcatatctcttgcttttcttgaatccaaatgcttaggacgtgtaaccttcacaaaccgtcataaatcagtttcgattcactacatgttgtagttattcaggctcttataattgaatattcaaaaatcgaattttctattttatcttgatcagaggatataactcgcttaaaatgcacaattttattagcgacaccatgtggatttgttttaattgaaacctctatcttctcatatgtatcacgatataaaatgctcattttattgactgtttaacgataaacttgcgacgatcgacgcaccaccgacctaattccattgtggaagcaaggggcactgcaataccgaaatgtgaagtaaatttcaaatctgctattatagacgatgatctaatactcttgaatggtcttaaacgtgagttgtatttcaagaagttatgacgattcgattttggggccattatgaccccaaaacccagccaacgtaacttttattagtacagacagaaggtcaagcgtgcaagtctttcatccgtgtgtcaataaggccatcagttgaaaccgtgtcaattaaccctccagttaacccttttaacttttaccaggacaaaccaatgacttcgtgcgcaaattccaccactcgttgtctcaggccttgagttgttgtttgataagaatgggggatgtcaagtcggggagcgtagcccaacaggctacggaaactgcatgatggcagtgtttgatccagggcactgttgggaatagactccgtcgaccgaagatcccagatgtcctgaaactcaataataagcgttagcagttacaaaatgggagcacccaggaagtgagtgacacccgatcgatacctcggaaacagtcccaaccggtaagaacccccataccttcgtcaatccgttggcgcgctttattgacgtctccgtcggcgcctttcagtatcacgtacatcaggggcatcatctcccaggggtatcgcagcttctccaggagccattgagatcgtttgacaagttcgtcgtccatggtggcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggctagcagatctcatatgattaagaggcgcggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacgctcgcgcgacttggtttgccattctttagcgcgcgtcgcgtcacacagcttggccacaatgtggatgtcgacttaa

SEQ ID NO: 27 - DNA, 1014 bp, Ceratitis capitata
  source 1..1014: mol_type = unassigned DNA
atggtcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcctacagccgcgcccgcaccaagaacaactacggcagcaccatcgagggcctgctggatctgccggatgatgatgccccggaggaggcgggcctggccgccccgcgcctgagcttcctgccggccggacacacccgccgcctgtcgaccgccccgccgaccgacgtgagcctgggcgatgagctgcacctggatggcgaggatgtggcgatggcccacgccgatgccctggacgacttcgacctggacatgctgggcgatggcgatagcccgggaccgggattcaccccgcacgatagcgccccctacggcgccctggatatggccgatttcgagttcgagcagatgttcaccgacgccctgggcatcgatgagtacggcggc

SEQ ID NO: 28 - DNA, 1008 bp, Ceratitis capitata
  source 1..1008: mol_type = unassigned DNA
atggtcagccgcctggacaagagcaaggtgatcaacagcgccctggagctgctgaacgaagtgggcatcgagggcctgaccacccgcaagctggcccagaagctgggcgtggaacagccgaccctgtactggcacgtgaagaacaagcgcgccctgctggacgccctggccatcgaaatgctggatcgccaccacacccacttctgcccgctggagggcgagagctggcaggatttcctgcgcaacaacgccaagagcttccgctgcgccctgctgtcgcaccgcgatggcgccaaggtgcacctgggcacccgcccgaccgagaagcagtacgagaccctggagaaccagctggccttcctgtgccagcagggcttcagcctggagaacgccctgtacgccctgagcgccgtgggccacttcaccctgggctgtgtgctggaggatcaggagcaccaggtggccaaggaggagcgcgagaccccgaccaccgatagcatgccgccgctgctgcgccaggccatcgagctgttcgatcaccagggcgccgagccggccttcctgttcggcctggagctgatcatctgcggcctggaaaagcagctgaagtgcgagagcggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcggg

SEQ ID NO: 29 - protein, 337 aa, Ceratitis capitata
VSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSGPAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 30 - protein, 238 aa, Dictyosoma burgeri
MASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFLRSRPKKKRKVEDP

SEQ ID NO: 31 - protein, 20 aa, synthetic construct
  REGION 1..20: note = Artificial leader peptide
MGTARVDPTPPKKKRKPVAT

SEQ ID NO: 32 - protein, 76 aa, Ceratitis capitata
MDDELVKRSQWLLEKLRYPWEMMPLMYVILKGADGDVNKARQRIDEGQAVVNEYSRLHNLNMFDGVELRSTTRQSG

SEQ ID NO: 33 - protein, 665 aa, synthetic construct
  REGION 1..665: note = Fusion of Drosophila melanogaster Ubiquitin with Ceratitis capitata tTAV
MDDELVKRSQWLLEKLRYPWEMMPLMYVILKGADGDVNKARQRIDEVQMLFNDNSDMQPFWGYHVSSTCETAFLKSVKVQTSDETNSAKQVSEGKFKKCKTAVVLNFEDCVSKAEVRLFHLPPEKNDDSRIINKQHPSEKIRETGSSTNHPTIYKPEIISLQSFPTSVGFGAQNDLINTYLSVACMPLRAYFGSWPLPNGSDRIRHLLHLLSEAFQQTQTLAGRGEHLRHPHSVCVTVAMCRFSYPVTVQLAGQIFVKTLTGKTITLEVEPSDTIENVKAKIQDKEGIPPDQQRLIFAGRQLEDGRTLSDYNIQKESTLHLVLRLRGGVSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSGPAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 34 - protein, 337 aa, Ceratitis capitata; identical to SEQ ID NO: 29

SEQ ID NO: 35 - protein, 103 aa, Ceratitis capitata
MDDELVKRSQWLLEKLRYPWEMMPLMYVILKGADGDVNKARQRIDEGYDICCIYFPKRSSRHRHWPDEENISVTRTPSASRSPCADFRTRSQSSSPDRSSSRP

SEQ ID NO: 36 - DNA, 1627 bp, synthetic construct
  misc_feature 1..1627: note = Insertion of synthetic contruct in Aedes aegypti chromosome
  source 1..1627: mol_type = other DNA
cttcaaagtctcatccccattgccataaagcctattcagtttccccaaaagtgtactgaaacagtgattattttaaacctccgcctatagttaaatttcggtgcgacattccacgataaatacatttcaggcagaatttgacgtcatagtcccagtatttcagcgataagactcatttgtacttccgtgagatgtttctataatatgaaaacactgataaataattaaatttaaaaaaaaaatccattagataaaattttacgctgttgctgcgcacgaaacacagttgctggtttgaaaagttatcaaaatgcgttgcattgttattcaatgaatggaatactcaagtagacttgttagatgtttcaaagatgctgtatttagaaagaataaacacatcttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattacagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatgaaaaaagagaacacccggcacggtaaaatgtcaaaaaagtggacttgcaatttcatttacagtgaaacctccatgagtcgatattgaagggaccatcgactcatggaaatatcgagtcatggaacaacaatcctttggaaagctgtttctagggaccatcatagtaaccatgaaattttgtttttagtatggttccatgagtcgatatcgagtcatggaacatcgactcatggaggtatcactgtactatcgttcttgcttgacccaatctcaccgctatttttgatgttttaaacactgtaccgagaaaaatgatttttttaaggaaaaatcaaatagattccggaaaatacgtgtgaagtgtaatgaaaagactttcaaccttctactaatagaacgatagaggttaaagtacatgcgtgatactttgaacaggagaaatttaatgttgtaaattttctctagtttcaaaatagtttattgatatagtaaacaaatactactatttctagaaatgtgtattgtgataaattacgtgtaataatatgttccctaa

SEQ ID NO: 37 - DNA, 1399 bp, synthetic construct
  misc_feature 1..1399: note = Synthetic construct inserted into the Aedes aegypti chromosome
  source 1..1399: mol_type = other DNA
gaccctaaattcaaaaacagataccaggcactctctctatatgtttcctccagattacgaagattgataatattttgaaagaataataaattgaaaaaaatcattatgttcacatcgatgagtgataaaaagccgtattaaaaaaaattacgatgatatttcaaatgaataataaattcactagagcagcattttttagtcaatgtgcaaaattattttgaaaaagcttattgatataaccatctaactctactttgtgtgggaatcatttcacatttgccggtcaaaaactcaaaaggcctgttttaataagcattttccatagctttctggtgtacggaatagaggtcaaaagtgaggaagcgtccgaaataagaaccatgtaaaggccacaagtttctattaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattacagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatataaaattatgcatgatgcggaagcgtaatctttacccacctttgaaaattcagtgtttctgcttaatgaagctgagttattacacaaaattattttatgtgctctgtgcagagttatacttatgagaccttaaataactgttatatgactcagaacggtattttcatagcttagaacgccaacaaatagatgtttaaagctaataaataggattatataaaaattcccattctttcatccaacatcatttcataaatcaaatttttcaaagaagctgtgcatcagttgtatttcgtggaacggatctggtgtagtgattagcacac

SEQ ID NO: 38 - DNA, 21 bp, synthetic construct
  misc_feature 1..21: note = Synthetic Primer
  source 1..21: mol_type = other DNA
ctgttgctgcgcacgaaacac

SEQ ID NO: 39 - DNA, 23 bp, synthetic construct
  misc_feature 1..23: note = Synthetic primer
  source 1..23: mol_type = other DNA
gtcagtcagaaacaactttggca

SEQ ID NO: 40 - DNA, 23 bp, synthetic construct
  misc_feature 1..23: note = Synthetic primer
  source 1..23: mol_type = other DNA
gcttcattaagcagaaacactga

SEQ ID NO: 41 - DNA, 23 bp, synthetic construct
  misc_feature 1..23: note = Synthetic Primer
  source 1..23: mol_type = other DNA
tcagtgtttctgcttaatgaagc

SEQ ID NO: 42 - DNA, 404 bp, Aedes aegypti
  source 1..404: mol_type = unassigned DNA
cttcaaagtctcatccccattgccataaagcctattcagtttccccaaaagtgtactgaaacagtgattattttaaacctccgcctatagttaaatttcggtgcgacattccacgataaatacatttcaggcagaatttgacgtcatagtcccagtatttcagcgataagactcatttgtacttccgtgagatgtttctataatatgaaaacactgataaataattaaatttaaaaaaaaaatccattagataaaattttacgctgttgctgcgcacgaaacacagttgctggtttgaaaagttatcaaaatgcgttgcattgttattcaatgaatggaatactcaagtagacttgttagatgtttcaaagatgctgtatttagaaagaataaacacatcttaa

SEQ ID NO: 43 - DNA, 558 bp, Aedes aegypti
  source 1..558: mol_type = unassigned DNA
tgaaaaaagagaacacccggcacggtaaaatgtcaaaaaagtggacttgcaatttcatttacagtgaaacctccatgagtcgatattgaagggaccatcgactcatggaaatatcgagtcatggaacaacaatcctttggaaagctgtttctagggaccatcatagtaaccatgaaattttgtttttagtatggttccatgagtcgatatcgagtcatggaacatcgactcatggaggtatcactgtactatcgttcttgcttgacccaatctcaccgctatttttgatgttttaaacactgtaccgagaaaaatgatttttttaaggaaaaatcaaatagattccggaaaatacgtgtgaagtgtaatgaaaagactttcaaccttctactaatagaacgatagaggttaaagtacatgcgtgatactttgaacaggagaaatttaatgttgtaaattttctctagtttcaaaatagtttattgatatagtaaacaaatactactatttctagaaatgtgtattgtgataaattacgtgtaataatatgttccctaa

SEQ ID NO: 44 - DNA, 406 bp, Aedes aegypti
  source 1..406: mol_type = unassigned DNA
gaccctaaattcaaaaacagataccaggcactctctctatatgtttcctccagattacgaagattgataatattttgaaagaataataaattgaaaaaaatcattatgttcacatcgatgagtgataaaaagccgtattaaaaaaaattacgatgatatttcaaatgaataataaattcactagagcagcattttttagtcaatgtgcaaaattattttgaaaaagcttattgatataaccatctaactctactttgtgtgggaatcatttcacatttgccggtcaaaaactcaaaaggcctgttttaataagcattttccatagctttctggtgtacggaatagaggtcaaaagtgaggaagcgtccgaaataagaaccatgtaaaggccacaagtttctattaa

SEQ ID NO: 45 - DNA, 328 bp, Aedes aegypti
  source 1..328: mol_type = unassigned DNA
tataaaattatgcatgatgcggaagcgtaatctttacccacctttgaaaattcagtgtttctgcttaatgaagctgagttattacacaaaattattttatgtgctctgtgcagagttatacttatgagaccttaaataactgttatatgactcagaacggtattttcatagcttagaacgccaacaaatagatgtttaaagctaataaataggattatataaaaattcccattctttcatccaacatcatttcataaatcaaatttttcaaagaagctgtgcatcagttgtatttcgtggaacggatctggtgtagtgattagcacac

SEQ ID NO: 46 - DNA, 2880 bp, Aedes aegypti
  source 1..2880: mol_type = unassigned DNA
aaataggaaaagtttttcatgggggtattcttctagaaagttgaagatacaatgacaaggaatgtatctagtactaaacatattgttatttttattaccatgtggttcatgtaacagttgtcagttcagcgccattttcaacttgcatgacaaattgtgacacgtttcacgtctttaattgactcgcaaaaaataaatgtgttttaaatcaaatttccatcagtaagctataatttttagtattattacaagctgctaacgataaaccagtattttgttttcatttcatatgaaattttcgatggtctatcttaccccaaaatatatttgtacctggggtaagtgggacctatcaaaacaaaaaccagaatcaaaacctttcgtacacgtttcatacatttagctagagagtagcactaaaacattcaaacaaatgatttttatcaaaaaagatgaacctcaaattacgtaattgcataaaagggagaaaaaaagtgttattattctttattatttaattattttttatttttgaaatacgacttcaaaattgaacaaacactcagctgaaatttgaaatgtatcatgagaacgattacttttctatgttgtttgaactttatttgttatttctaccaaattaagtagtgatggaaaacaattccatcccataaggtggttttctgccatgcatataaataataacaaaacatatttataatttcgtcaattattgattgtttatgagctacatttttattaacaacttataaatgatatattttgaacatgtttaattcctctttacgcatttattgaggaattttcagttaatattaaatatgaggtgacatactattaaattgagggtttcttcctaaaacgtaacgtattttatggttgatcccttatgtacatcaaatatgtacttaaaattaaaaatctattactgtaatagttgacttactgatgtggattgacgcatgaaactggatgtgtcccacttgccccgcttagagaggtaattgcgtccattcgctcattctaaaactttcttctaaggttttcacaatttcgaaattattcgattagtttcatgcacacaccagcaaatatgttgccaaaacgtgattgtgttgttggaattgaaaaatattgacatttggaaattttattgaacaatttgtttgaactatcgtttttttttcgttcccacttgccccgcggtaccttaacttgtttttgaaaacatgatttcaaaaccattcacaactgaaaaatattgacaaaaaacgatgcaaacaagaaaaaaatatctaagatgtttcacaagtatgataaacccacttaaaagaaagattatactgaaaattgaagagctatgagaaaaaatatgtaaacccaacatttatcgtcaattcttggtttttccctcaaattgtcttcaaagtctcattcccattgctataaagcccattcagtttccccaaaggtgtactgaaacagtgattattttaaaccttcgcaaatagttaaatttcggtgcgacattccacgatcaataaatttcaggcagaagttgacgtcataattccagtatttcagcgatccaactcatttgtacttccgtgagatgtttctataatataaaaacactgataaataatcaaatttagaaaaaaaaacacctttttgataaaaatttacggtgttgctgcgcacgaaacacagttgctggtttgagaagttgtcaaaatgcgttgtattgttattcaatgcacggaatactcaagtagacttgtttgatgtttcagagatgctgtatttagaaagaataaacacatcttaatgaaaaaagagaacacccggcaccgtaaaatgtcaaaaaagtggacttggaattttatttactatcgttcttgcttgatccaatctcacccccatttccaatgttttagacatcgtgccgaaacaaatgatttttttgtgggaaaatcaaacagattccggaaaatacctgtgatgtgtaatgaaaagactttcaacattctactaatacaacgatagaggctagagtacatgcgtgatactttgacaggagaaatttaatgttgaaaattttatttagtttcaacatgcaagtttattgctgtagtacacaaaaactactatttctaaaaatgtatattgttatgcattatgtgtaataatatgttccctaacatatgaattattaattttagtaatatctgaaatatttcacaaaacatatttttccgttttgacccaatctcacccccatcgacggtattcagttctttcaaattatatgaatacattaaaatcagatctctaccatcatttctttaactcattcggccgaatgaccctttcatcttaatgatttatcggtctgatgtgttttgtcctaacgaacttctgcttaacgactcaacaccgtgaacttctgagtaatccacgaagattgtatttaatatttttatttatttatttatttatttttttttttttttttgaagcactgcggaaataatccctaggtgaaaatgaaatgctagataaatacttatggtggtaacctccattagggaaatacccccagacaaccagaagtcgcatgaaagctcacgtaataactcgttaattcctacaaattacatcaaacccgcaaaacacggtgcataaaatcgccagattgatgagtttcctcgcgaaaaacgatttttttttctgggttcatttgtacattttgtttcgttccgtacactcgtacaaagtaagtcgaattaatccgtagaagctgtcaaatcagcatttgttatcataagttaagtcgcatatgaccacaggttagttcgg

SEQ ID NO: 47 - DNA, 17 bp, synthetic construct
  misc_feature 1..17: note = Synthetic Oligonucleotide
  source 1..17: mol_type = other DNA
catgccgacgcgctaga

SEQ ID NO: 48 - DNA, 27 bp, synthetic construct
  misc_feature 1..27: note = Synthetic Oligonucleotide
  source 1..27: mol_type = other DNA
ggtaaacatctgctcaaactcgaagtc

SEQ ID NO: 49 - DNA, 24 bp, synthetic construct
  misc_feature 1..24: note = Synthetic Oligonucleotide
  misc_feature 1: note = FAM fluorescent label
  misc_feature 24: note = BHQ-1 quencher
  source 1..24: mol_type = other DNA
tcgatctggacatgttgggggacg

SEQ ID NO: 50 - DNA, 25 bp, Aedes aegypti
  source 1..25: mol_type = unassigned DNA
ctgcagtagtgatgaagatgaacca

SEQ ID NO: 51 - DNA, 25 bp, Aedes aegypti
  source 1..25: mol_type = unassigned DNA
gggcgaaaatgccgtattgtactca

SEQ ID NO: 52 - DNA, 27 bp, Aedes aegypti
  misc_feature 1: note = HEX Fluorescent Label
  misc_feature 27: note = BHQ-1 Quencher
  source 1..27: mol_type = unassigned DNA
agacaccagtcggacttgcaaaatctg

SEQ ID NO: 53 - DNA, 23 bp, Aedes aegypti
  source 1..23: mol_type = unassigned DNA
gcttcattaagcagaaacactga

SEQ ID NO: 54 - DNA, 28 bp, Aedes aegypti
  source 1..28: mol_type = unassigned DNA
catctaactctactttgtgtgggaatca

SEQ ID NO: 55 - DNA, 22 bp, Aedes aegypti
  source 1..22: mol_type = unassigned DNA
tgacaagcacgcctcacgggag

SEQ ID NO: 56 - DNA, 21 bp, synthetic construct
  misc_feature 1..21: note = Oligonucleotide Primer
  source 1..21: mol_type = other DNA
ctgttgctgcgcacgaaacac

SEQ ID NO: 57 - DNA, 24 bp, synthetic construct
  misc_feature 1..24: note = Oligonucleotide Primer
  source 1..24: mol_type = other DNA
gtgccaaagttgtttctgactgac

SEQ ID NO: 58 - DNA, 24 bp, synthetic construct
  misc_feature 1..24: note = Oligonucleotide Primer
  source 1..24: mol_type = other DNA
gatggtccctagaaacagctttcc

SEQ ID NO: 59 - DNA, 21 bp, synthetic construct
  misc_feature 1..21: note = Oligonucleotide Primer
  source 1..21: mol_type = other DNA
ctgttgctgcgcacgaaacac

SEQ ID NO: 60 - DNA, 25 bp, synthetic construct
  misc_feature 1..25: note = Oligonucleotide Primer
  source 1..25: mol_type = other DNA
tcgatcaactaactgaaatcgatga

SEQ ID NO: 61 - DNA, 27 bp, synthetic construct
  misc_feature 1..27: note = Oligonucleotide Primer
  source 1..27: mol_type = other DNA
cctaagaccgttaacatttcaagtgac

SEQ ID NO: 62 - DNA, 23 bp, synthetic construct
  misc_feature 1..23: note = Oligonucleotide Primer
  source 1..23: mol_type = other DNA
cttcgagagtaagcggaaactcc

SEQ ID NO: 63 - DNA, 25 bp, synthetic construct
  misc_feature 1..25: note = Oligonucleotide Primer
  source 1..25: mol_type = other DNA
agtattagcatccgaagctcatgac

SEQ ID NO: 64 - DNA, 23 bp, synthetic construct
  misc_feature 1..23: note = Oligonucleotide Primer
  source 1..23: mol_type = other DNA
gcttcattaagcagaaacactga

SEQ ID NO: 65 - DNA, 24 bp, synthetic construct
  misc_feature 1..24: note = Oligonucleotide Primer
  source 1..24: mol_type = other DNA
cagaccgataaaacacatgcgtca

SEQ ID NO: 66 - DNA, 22 bp, synthetic construct
  misc_feature 1..22: note = Oligonucleotide Primer
  source 1..22: mol_type = other DNA
tcgactcatggaggtttcactg

SEQ ID NO: 67 - DNA, 23 bp, synthetic construct
  misc_feature 1..23: note = Oligonucleotide Primer
  source 1..23: mol_type = other DNA
atgcgttgcattgttattcaatg

SEQ ID NO: 68 - DNA, 25 bp, synthetic construct
  misc_feature 1..25: note = Oligonucleotide Primer
  source 1..25: mol_type = other DNA
aaatatcagcctcaaataagcactt

SEQ ID NO: 69 - DNA, 28 bp, synthetic construct
  misc_feature 1..28: note = Oligonucleotide Primer
  source 1..28: mol_type = other DNA
ctatagctttctggtgtacggaatagag

SEQ ID NO: 70 - DNA, 28 bp, synthetic construct
  misc_feature 1..28: note = Oligonucleotide Primer
  source 1..28: mol_type = other DNA
ggtctcataagtataactctgcacagag

SEQ ID NO: 71 - DNA, 24 bp, synthetic construct
  misc_feature 1..24: note = Oligonucleotide Primer
  source 1..24: mol_type = other DNA
tgcagtagtgatgaagatgaacca

SEQ ID NO: 72 - DNA, 22 bp, synthetic construct
  misc_feature 1..22: note = Oligonucleotide Primer
  source 1..22: mol_type = other DNA
cgaaaatgccgtattgtactca

SEQ ID NO: 73 - DNA, 402 bp, Aedes aegypti
  source 1..402: mol_type = unassigned DNA
cttcaaagtctcatccccattgccataaagcctattcagtttccccaaaagtgtactgaaacagtgattattttaaacctccgcctatagttaaatttcggtgcgacattccacgataaatacatttcaggcagaatttgacgtcatagtcccagtatttcagcgataagactcatttgtacttccgtgagatgtttctataatatgaaaacactgataaataattaaatttaaaaaaaaaatccattagataaaattttacgctgttgctgcgcacgaaacacagttgctggtttgaaaagttatcaaaatgcgttgcattgttattcaatgaatggaatactcaagtagacttgttagatgtttcaaagatgctgtatttagaaagaataaacacatctt

SEQ ID NO: 74 - DNA, 560 bp, Aedes aegypti
  source 1..560: mol_type = unassigned DNA
aatgaaaaaagagaacacccggcacggtaaaatgtcaaaaaagtggacttgcaatttcatttacagtgaaacctccatgagtcgatattgaagggaccatcgactcatggaaatatcgagtcatggaacaacaatcctttggaaagctgtttctagggaccatcatagtaaccatgaaattttgtttttagtatggttccatgagtcgatatcgagtcatggaacatcgactcatggaggtatcactgtactatcgttcttgcttgacccaatctcaccgctatttttgatgttttaaacactgtaccgagaaaaatgatttttttaaggaaaaatcaaatagattccggaaaatacgtgtgaagtgtaatgaaaagactttcaaccttctactaatagaacgatagaggttaaagtacatgcgtgatactttgaacaggagaaatttaatgttgtaaattttctctagtttcaaaatagtttattgatatagtaaacaaatactactatttctagaaatgtgtattgtgataaattacgtgtaataatatgttccctaa

SEQ ID NO: 75 - DNA, 404 bp, Aedes aegypti
  source 1..404: mol_type = unassigned DNA
gaccctaaattcaaaaacagataccaggcactctctctatatgtttcctccagattacgaagattgataatattttgaaagaataataaattgaaaaaaatcattatgttcacatcgatgagtgataaaaagccgtattaaaaaaaattacgatgatatttcaaatgaataataaattcactagagcagcattttttagtcaatgtgcaaaattattttgaaaaagcttattgatataaccatctaactctactttgtgtgggaatcatttcacatttgccggtcaaaaactcaaaaggcctgttttaataagcattttccatagctttctggtgtacggaatagaggtcaaaagtgaggaagcgtccgaaataagaaccatgtaaaggccacaagtttctatt

SEQ ID NO: 76 - DNA, 206 bp, Aedes aegypti
  source 1..206: mol_type = unassigned DNA
aatataaaattatgcatgatgcggaagcgtaatctttacccacctttgaaaattcagtgtttctgcttaatgaagctgagttattacacaaaattattttatgtgctctgtgcagagttatacttatgagaccttaaataactgttatatgactcagaacggtattttcatagcttagaacgccaacaaatagatgtttaaagcta

SEQ ID NO: 77 - DNA, 23 bp, synthetic construct
  misc_feature 1..23: note = Oligonucleotide primer
  source 1..23: mol_type = other DNA
aaatgaaattgcaagtccacttt

SEQ ID NO: 78 - DNA, 23 bp, synthetic construct
  misc_feature 1..23: note = Oligonucleotide primer
  source 1..23: mol_type = other DNA
gcgtcaattttacgcagactatc

SEQ ID NO: 79 - DNA, 23 bp, synthetic construct
  misc_feature 1..23: note = Oligonucleotide primer
  misc_feature 1: note = FAM Fluorescent Label
  misc_feature 23: note = BHQ-1 Quencher
  source 1..23: mol_type = other DNA
acacccggcacggtaaaatgtca

SEQ ID NO: 80 - DNA, 24 bp, synthetic construct
  misc_feature 1..24: note = Oligonucleotide primer
  source 1..24: mol_type = other DNA
agcagaaacactgaattttcaaag

SEQ ID NO: 81 - DNA, 23 bp, synthetic construct
  misc_feature 1..23: note = Oligonucleotide primer
  source 1..23: mol_type = other DNA
gcgtcaattttacgcagactatc

SEQ ID NO: 82 - DNA, 27 bp, synthetic construct
  misc_feature 1..27: note = Oligonucleotide primer
  misc_feature 1: note = FAM Fluorescent label
  misc_feature 27: note = BHQ-1 Quencher
  source 1..27: mol_type = other DNA
atgatgcggaagcgtaatctttaccca

SEQ ID NO: 83 - DNA, 24 bp, synthetic construct
  misc_feature 1..24: note = Oligonucleotide primer
  source 1..24: mol_type = other DNA
tgcagtagtgatgaagatgaacca

SEQ ID NO: 84 - DNA, 22 bp, synthetic construct
  misc_feature 1..22: note = Oligonucleotide primer
  source 1..22: mol_type = other DNA
cgaaaatgccgtattgtactca

SEQ ID NO: 85 - DNA, 27 bp, synthetic construct
  misc_feature 1..27: note = Oligonucleotide primer
  misc_feature 1: note = HEX Fluorescent label
  misc_feature 27: note = BHQ-1 Quencher
  source 1..27: mol_type = other DNA
agacaccagtcggacttgcaaaatctg

(19) United States
(12) Patent Application Publication
(10) Pub. No.: US 2024/0093230 A1
(43) Pub. Date: Mar. 21, 2024

(54) SELF-LIMITING, SEX-SPECIFIC GENE AND METHODS OF USING

(71) Applicant: Oxitec Ltd., Abingdon (GB)

(72) Inventors: Luke ALPHEY, Pirbright (GB); Tarig DAFA'ALLA, Abingdon (GB); Amandine COLLADO, Abingdon (GB); Simon WARNER, Abingdon (GB); Kelly MATZEN, Abingdon (GB); Sian SPINNER, Abingdon (GB)

(73) Assignee: Oxitec Ltd., Abingdon (GB)

(21) Appl. No.: 18/489,609

(22) Filed: Oct. 18, 2023

Related U.S. Application Data
(62) Division of application No. 16/324,078, filed on Feb. 7, 2019, now Pat. No. 11,827,876.
(60) Provisional application No. 62/374,415, filed on Aug. 12, 2016.
(60) Provisional application No. 62/420,270, filed on Nov. 10, 2016.

(51) Int. Cl. C12N 15/85; A01K 67/033

(52) U.S. Cl. CPC C12N 15/8509; A01K 67/0339; A01K 2227/706; A01K 2267/02; C12N 2830/003; C12N 2840/44

(57) ABSTRACT

The invention provides a splice control module for sex-specific splicing and expression of a gene of interest. In certain embodiments, a dsx-based splice control module is used to express a lethal gene in an insect that is spliced in a sex-specific manner to impart lethality to female insects but not male insects.

### CROSS-REFERENCE TO RELATED APPLICATIONS

This application is a divisional of U.S. application Ser. No. 16/324,078, filed on Feb. 7, 2019, which is a U.S. national phase of International Patent Application No. PCT/IB2017/001128, filed Aug. 9, 2017, which claims the benefit of priority to U.S. provisional patent applications 62/374,415, filed Aug. 12, 2016, entitled “A SELF-LIMITING, SEX-SPECIFIC GENE AND METHODS OF USING,” 62/420,270, filed Nov. 10, 2016, entitled “A SELF-LIMITING, SEX-SPECIFIC GENE AND METHODS OF USING” and the contents of which are hereby incorporated by reference in their entirety for all purposes.

### REFERENCE TO AN ELECTRONIC SEQUENCE LISTING

The contents of the electronic sequence listing (166372001610SEQLIST.xml; Size: 135,743 bytes; and Date of Creation: Oct. 17, 2023) is herein incorporated by reference in its entirety.

### BACKGROUND OF THE INVENTION

Alternative splicing plays a key role in the regulation of gene expression in many developmental processes ranging from sex determination to apoptosis (Black, D. L. (2003) Annu. Rev. Biochem. 72, 291-336), and defects in alternative splicing have been linked to many human disorders (Caceres, J. F. & Kornblihtt, A R. (2002) Trends Genet. 18, 186-193). In general, alternative splicing is regulated by proteins that associate with the pre-mRNA and function to either enhance or repress the ability of the spliceosome to recognize the splice site(s) flanking the regulated exon (Smith, C. W. & Valcarcel, J. (2000) Trends Biochem. Sci. 25, 381-388).

Alternative splicing involves the removal of one or more introns and ligation of the flanking exons. This reaction is catalyzed by the spliceosome, a macromolecular machine composed of five RNAs, including small nuclear RNA and protein particles (snRNPs) which assemble with pre-mRNA to achieve RNA splicing, by removing introns from eukaryotic nuclear RNAs, thereby producing mRNA which is then translated to protein in ribosomes (Jurica, M. S. & Moore, M. J. (2003) Mol. Cell 12, 5-14; Smith, C. W. & Valcarcel, J. (2000) Trends Biochem. Sci. 25, 381-388). Alternative splicing generates multiple mRNAs from a single gene, thus increasing proteome diversity (Graveley, B. R. (2001) Trends Genet. 17, 100-107).

Whether a particular alternative exon will be included or excluded from a mature RNA in each cell is thought to be determined by the relative concentration of a number of positive and negative splicing regulators and the interactions of these factors with the pre-mRNA and components of the spliceosome (Smith, C. W. & Valcarcel, J. (2000) Trends Biochem. Sci. 25, 381-388).

Dengue fever is a viral disease primarily transmitted by the mosquito, Aedes aegypti, with an estimated incidence of 390 million infections annually (Bhatt et al., 2013). With no specific drugs available and a limited distribution of the licensed vaccine, Dengvaxia● (Villar et al, 2015, Constenia & Clark, 2016), efforts to reduce transmission depend predominantly on insecticide based vector control (WHO-TDR, 2009). Aedes aegypti also transmits other dangerous diseases such as yellow fever, chikungunya, and Zika. With the potential for the spread of insecticide resistance, the development of transgenic vectors may provide an effective method to limit the transmission of the disease by reducing the density or vectoral capacity of the vector population.

We have developed and tested a self-limiting technology that confers a repressible phenotype whereby, in the absence of a tetracycline analogue, all mosquitoes carrying a copy of the transgene die at an early larval stage due to the accumulation of tTAV protein produced by a positive feedback loop. Male mosquitoes, which do not bite or transmit disease, are selected and released to mate with wild females and therefore, the progeny, which inherit the self-limiting gene do not survive to adulthood due to the lack of tetracycline in the environment. In generating mosquitoes for release, the larvae and pupae are grown in the presence of tetracycline, wherein the mosquito pupae can mature to adulthood. However, in order to select only males, the pupae must be sorted by sex before eclosion.

Currently, the sex separation in Aedes mosquitoes is being done with a manual/mechanical procedure. While the procedure is very effective, it is extremely labour-intensive and human error can result in sexing errors. It is an inefficient method in medium to large-scale operational programs. We have pioneered in the development of mechanical sex sorters and methods for sorting larvae from pupae to facilitate the sex sort at scale, but these too require people and quality control to ensure efficient and accurate male production. Early and non-labour-intensive elimination of females could further enhance the cost saving benefit as potentially twice as many males could be produced from the same rearing environment as is currently possible.

There is a need in the art for a self-selecting separation procedure to increase the accuracy and efficiency of male/female separation.

### BRIEF SUMMARY OF THE INVENTION

The invention provides a splice control module for differentially expressing a gene of interest in an organism.

The invention provides a doublesex (dsx) splice control module polynucleotide comprising, from 5′ to 3′:
a. an exon 4 of dsx;
b. a truncated intron 4 of dsx comprising a 5′ terminal fragment of the dsx intron 4 and a 3′ fragment of the dsx intron 4;
c. an exon 5a of dsx;
d. an intron 5 of dsx;
e. a modified exon 5b of dsx;

f a truncated intron 6 of dsx comprising a 5′ terminal fragment of the dsx intron 6 and a 3′ fragment of the dsx intron 6; and
g. a 5′ fragment of exon 6.

In some embodiments, the dsx splicing is derived from Aedes aegypti (Aeadsx).

In some embodiments, the dsx splice control module has a modified exon 5b in which an open reading frame is created for the entire exon. In some embodiments, the modified exon 5b comprises at least one substitution, insertion, and/or deletion to form an open reading frame for the entire exon.

The invention provides a dsx splice control module wherein splicing occurs on a sex-specific basis when introduced into an insect. In some embodiments, the insect is of the order selected from the group consisting of Diptera or Calliphoridae. In some embodiments, the insect is a dipteran selected from the group consisting of Medfly (Ceratitis capitata), Mexfly (Anastrepha ludens), Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata) Caribbean fruit fly (Anastrepha suspensa) or West Indian fruit fly (Anastrepha obliqua).

In some embodiments, the dipteran insect is a mosquito of a genera selected from the group consisting of Stegomyia, Aedes, Anopheles and Culex. In specific embodiments, the mosquito is a species selected from the group consisting of Stegomyia aegyptae (also known as Aedes aegypti), Stegomyia albopicta (also known as Aedes albopictus), Anopheles stephensi, Anopheles albimanus and Anopheles gambiae.

In other embodiments, the insect is a Calliphoridae insect selected from the group consisting of New world screwworm (Cochliomyia hominivorax), Old world screwworm (Chrysomya bezziana) and Australian sheep blowfly (Lucilia cuprina). In other embodiments, the insect is a Lepidoptera insect selected from the group consisting of codling moth (Cydia pomonella), silk worm (Bombyx mori), pink bollworm (Pectinophora gossypiella), diamondback moth (Plutella xylostella), the Gypsy moth (Lymantria dispar), Navel Orange Worm (Amyelois transitella), Peach Twig Borer (Anarsia lineatella) rice stem borer (Tryporyza incertulas), and noctuid moths (e.g., Heliothinae).

In other embodiments, the insect is a Coleoptera insect selected from the group consisting of Japanese beetle (Popillajaponica), white-fringed beetle (Graphognatus spp), boll weevil (Anthonomous grandis), corn root worm (Diabrotica spp.) and Colorado potato beetle (Leptinotarsa decernlineata).

In certain specific embodiments, the insect is the mosquito is Aedes aegypti.

The invention also provides a gene expression system comprising a polynucleotide comprising a doublesex (dsx) splice control module of the invention and a polynucleotide encoding heterologous protein. In some embodiments, the dsx splice control module is derived from Aedes aegypti (Aeadsx).

In some embodiments, the gene expression system of the invention further comprising a polynucleotide encoding a gene that is deleterious, lethal or sterilizing operably linked to 3′ of said splice control module. In some embodiments, the gene is a synthetic tetracycline repressive transcriptional activator protein (tTAV). In some embodiments, the gene expression system further comprises a polynucleotide sequence encoding a Fusion Leader Polypeptide (e.g., ubiquitin) fused in frame to the 5′ end of said polynucleotide encoding said tTAV. In some embodiments, the gene expression system further comprising a 5′ untranslated region (5′UTR) operably linked 5′ of said splice control module. In some embodiments, the 5′UTR comprises a promoter operable in an insect. In some embodiments, the promoter is a Drosophila melanogaster minimal HSP70 promoter (DmHsp70). In some embodiments, the 5′UTR further comprises a tetracycline responsive operator (e.g., TetOx7). In some embodiments, the gene expression system further comprising a 3′ untranslated region (3′UTR) operably linked 3′ of said tTAV. In some embodiments, the 3′UTR is an SV40 3′UTR.

The invention also provides an expression vector plasmid comprising a gene expression system of the invention. In some embodiments, the expression vector plasmid further comprises a polynucleotide encoding a fluorescent marker protein (e.g., DsRed2). In some embodiments, the polynucleotide encoding said fluorescent marker protein is operably linked to a promoter (e.g., an IE1 promoter). In some embodiments, the expression vector plasmid further comprises a piggyBac transposable element ends to direct incorporation of said expression vector plasmid into the chromosome of an organism.

The invention also provides a genetically engineered insect comprising a gene expression system incorporated into a chromosome of said insect, said gene expression system comprising a polynucleotide construct comprising:
a. a doublesex (dsx) splice control module wherein said splice control module comprises the components from 5′ to 3′:
b. an exon 4;
c. a truncated intron 4 of dsx comprising a 5′ terminal fragment of the dsx intron 4 and a 3′ fragment of the dsx intron 4;
d. an exon 5a;
e. an intron 5 of dsx;
f. a modified exon 5b of said dsx;
g. a truncated intron 6 of dsx comprising a 5′ terminal fragment of the dsx intron 6 and a 3′ fragment of the dsx intron 6; and
h. a 5′ fragment of exon 6;
i. a polynucleotide encoding ubiquitin fused in frame to the 5′ end of a polynucleotide encoding tTAV positioned 3′ of said splice control module; and
j. a 5′UTR positioned 5′ of said splice control module wherein said 5′UTR comprises a tetracycline responsive operator (TetO x7) and a promoter.

In some embodiments, the genetically engineered insect is a mosquito, such as one of a genera selected from the group consisting of Aedes, Anopheles, and Culex. In some embodiments, the mosquito is Aedes aegypti.

In some embodiments, the genetically engineered insect further comprises a polynucleotide encoding a fluorescent protein (e.g., DsRed2). In some embodiments, the fluorescent protein is operably linked to a promoter (e.g., an IE1 promoter).

The invention also provides a method of producing genetically engineered insects comprising modifying an insect's chromosome by inserting a gene expression system, wherein said gene expression system comprises:
a. a doublesex (dsx) splice control module wherein said splice control module comprises the components from 5′ to 3′:
b. an exon 4;
c. a truncated intron 4 of dsx comprising a 5′ terminal fragment of the dsx intron 4 and a 3′ fragment of the dsx intron 4;
d. an exon 5a;
e. an intron 5 of dsx;
f. a modified exon 5b of said dsx;
g. a truncated intron 6 of dsx comprising a 5′ terminal fragment of the dsx intron 6 and a 3′ fragment of the dsx intron 6; and
h. a 5′ fragment of exon 6;
i. a polynucleotide encoding ubiquitin fused in frame to the 5′ end of a polynucleotide encoding tTAV positioned 3′ of said splice control module; and
j. a 5′UTR positioned 5′ of said splice control module wherein said 5′UTR comprises a tetracycline responsive operator (TetO x7) and a promoter.

In some embodiments of the method of the invention, the insect is a mosquito of a genus selected from the group consisting of Aedes, Anopheles, and Culex. In some embodiments, the mosquito is Aedes aegypti.

In some embodiments of the method of the invention, the gene expression system further comprises a polynucleotide encoding a fluorescent protein (e.g., DsRed2). In some embodiments, the fluorescent protein is operably linked to a promoter (e.g., an IE1 promoter).

The invention also provides a method of selectively rearing male genetically engineered insects comprising, rearing a genetically engineered insect of the invention in the absence of tetracycline.

The invention also provides a genetically engineered male insect produced by the method of the invention.

The invention also provides a method of reducing a wild insect population comprising contacting said wild insect population with a plurality of the male genetically engineered insects of the invention wherein said male genetically engineered insects mate with wild female insects of the same species. In some embodiments, the insect is a mosquito of a genus selected from the group consisting of Aedes, Anopheles, and Culex. In some embodiments, the mosquito is Aedes aegypti.

### BRIEF DESCRIPTION OF THE DRAWINGS

FIG. 1 shows two gene cassettes are present in the rDNA of DSX-tTAV-Red. One expresses the fluorescent marker, DsRed2 and the other expresses the tTAV protein female-specifically. Exons are expressed as E4, E5a, E5b, and E6. Hr5/IE1 are the promoter/Enhancer from the Baculovirus Autographica californica nucleopolyhedrosisvirus (AcNPV), DmHSP70 is the HSP70 gene from Drosophila melanogaster.

FIG. 2 shows a plasmid map for DSX-tTAV-Red. The rDNA is shown by the thin black line running along the inside of the plasmid schematic. Components not designated as rDNA are not incorporated into the insect genome. A list and description of rDNA components are provided in Table 1.

FIG. 3 shows the splicing behaviour of the Aeadsx splice control module in DSX-tTAV-Red strains. The Aeadsx splice module consists of Aeadsx exons 4, 5a, 5b and 6, together with fragments of Aeadsx introns 4 and 5. The F1 transcript contains Aeadsx exon 4 joined to Aeadsx exon 5a and part of Aeadsx intron 5 (serves as a 3′UTR with an internal termination and polyadenylation (polyA) signal). This transcript has a short open reading frame (ORF) that starts immediately upstream to Aeadsx exon 4 and ends at a stop codon in Aeadsx exon 5a. F2 transcript contains Aeadsx exon 4, Aeadsx a modified exon 5b, a truncated Aeadsx exon 6, Ubiquitin and SV40 3′UTR. This transcript has a long ORF, starts upstream to Aeadsx exon 4 and ends immediately after the 3′ end of tTAV encoding sequence. Transcript M contains Aeadsx exon 4, Aeadsx exon 6, Ubiquitin, tTAV and SV40 3′UTR. The ORF in this transcript starts, as in the other two transcripts, upstream to Aeadsx exon 4 and ends in Ubiquitin sequence (in a frame different than the ORF coding for Ubiquitin protein). The arrow indicates the position of the start codon and the red stop sign indicates that of the stop codon.

FIG. 4 shows Images of DSX-tTAV-Red-O, OX513A and WT life stages expressing the DsRed2 marker under white light and fluorescent light. Panel A: wild type (wt) pupae and larva, DSX-tTAV-Red-O pupae and larva under white light; Panel B: the same pupae and larvae shown in A under fluorescent light; Panel C: OX513A larvae under white light; Panel D: the same larvae shown in C under fluorescent light.

FIG. 5 shows the proportion of functional DSX-tTAV-Red adults surviving from on-off doxycycline rearing of DSX-tTAV-Red-O and DSX-tTAV-Red-S. Percentages are means of individuals becoming functional adults, based on the number of fluorescent pupae collected per strain. 95% confidence intervals are displayed in parentheses for female samples. Male samples were pooled (see methods section).

FIG. 6 shows functional DSX-tTAV-Red adult female eclosion. Percentages are the means of fluorescent female pupae eclosing into functional adults. 95% confidence intervals are displayed. Of the fluorescent pupae collected from hemizygous crossed individuals, the ratio of hemizygotes to homozygotes is expected to be 2:1 (based on Mendelian genetics). Any adult eclosion over 50% indicates survival of homozygotes under permissive conditions.

FIG. 7 shows a summary of the criteria for the strain selection that resulted in DSX-tTAV-Red-O and DSX-tTAV-Red-S.

FIG. 8 is a representative gel showing PCR products using primers SS2326 and TD225)Mod-666-sal to amplify across the genomic DNA-transgene rDNA boundary in DSX-tTAV-Red-S. Expected amplicon size: 221 bp. Samples 13, 15, 37, and B6 represent individual mosquitoes screened for the presence of the DSX-tTAV-Red-S transgene insertion. Sample c-Fve' is a known DSX-tTAV-Red-S individual and ‘H₂O’ is a no-DNA negative control sample. M indicates molecular weight markers.

FIG. 9 presents a schematic figure showing detection methods for DSX-tTAV-Red rDNA in Aedes aegypti genomic DNA. Primer 1 anneals to DNA in the region flanking the insertion site of the DSX-tTAV-Red rDNA. Primer 2 anneals to DNA within the DSX-tTAV-Red rDNA. A Taqman probe annealing to the amplicon is also depicted, where ‘*’ represents a generic fluorophore and ‘Q’ represents a generic quencher in the Taqman probe. Primer 3 anneals to DNA in the region flanking the other end of the insertion site.

FIG. 10 shows relative copy number of the DSX-tTAV-Red rDNA detected in DSX-tTAV-Red-O homozygous individual mosquitoes (horn, in red), DSX-tTAV-Red-O hemizygous individuals (het, in blue) and wild-type individuals (WT). A no-DNA control reaction (NTC) was also carried out. These data were generated using the primers and probe outlined in Table 10. Relative copy number was calculated by first normalising Ct (cycle threshold) values to Ct values obtained for an endogenous Aedes aegypti gene (IAP1), and then normalising Ct values to DSX-tTAV-Red rDNA Ct values obtained for an DSX-tTAV-Red rDNA homozygous individual.

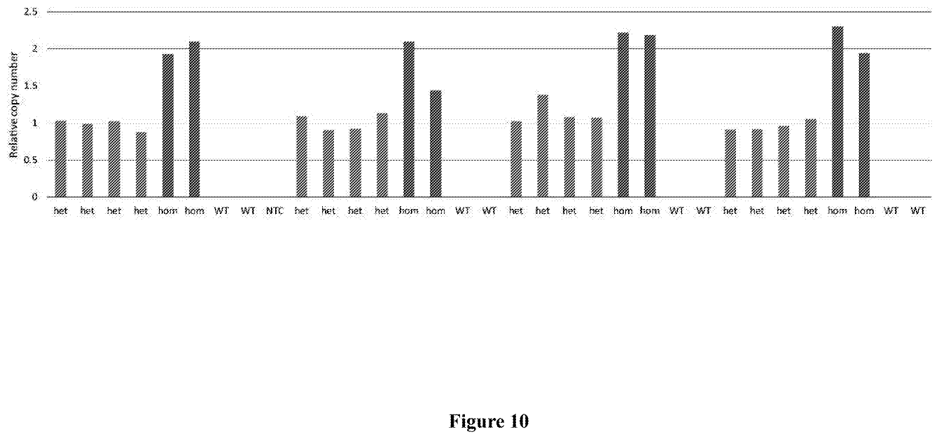

### DETAILED DESCRIPTION OF THE INVENTION

### Definitions

This description contains citations to various journal articles, patent applications and patents. These are herein incorporated by reference as if each was set forth herein in its entirety.

The term “penetrance,” as used herein, refers to the proportion of individuals carrying a particular variant of a gene that also express the phenotypic trait associated with that variant. Thus, “penetrance”, in relation to the present invention, refers to the proportion of transformed organisms which express the lethal phenotype.

The term “construct,” as used herein, refers to an artificially constructed segment of DNA for insertion into a host organism, for genetically modifying the host organism. At least a portion of the construct is inserted into the host organism's genome and alters the phenotype of the host organism. The construct may form part of a vector or be the vector.

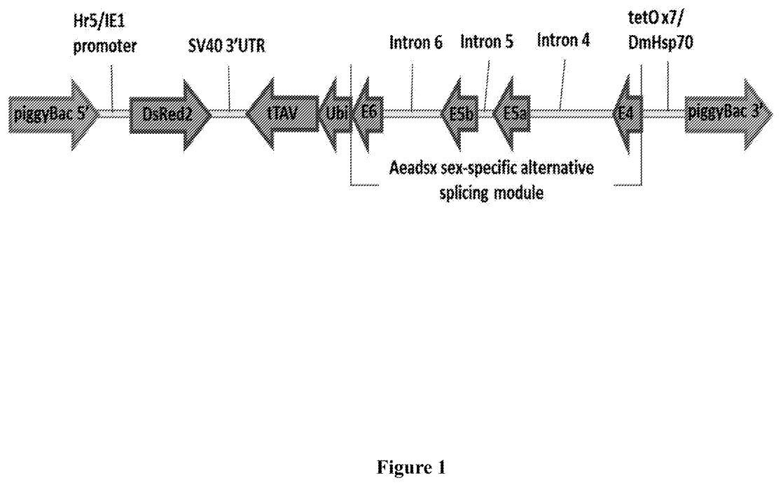

The term “transgene,” as used herein, refers to the polynucleotide sequence comprising a first and a second gene expression system to be inserted into a host organism's genome, to alter the host organism's phenotype. The portion of the plasmid vector containing the genes to be expressed (as shown in FIG. 1, for example) is referred to herein as the transfer DNA or recombinant DNA (rDNA).

The term “gene expression system,” as used herein, refers to a gene to be expressed together with any genes and DNA sequences which are required for expression of said gene to be expressed.

The term “splice control sequence,” as used herein, refers to a DNA sequence associated with a gene, wherein the DNA sequence, together with a spliceosome, mediates alternative splicing of a RNA product of said gene. Preferably, the splice control sequence, together with the spliceosome, mediates splicing of a RNA transcript of the associated gene to produce an mRNA coding for a functional protein and mediates alternative splicing of said RNA transcript to produce at least one alternative mRNA coding for a non-functional protein. A “splice control module” may contain multiple splice control sequences that join multiple exons to form a polypeptide encoding nucleic acid.

The term “transactivation activity,” as used herein, refers to the activity of an activating transcription factor, which results in an increased expression of a gene. The activating transcription factor may bind a promoter or operator operably linked to said gene, thereby activating the promoter and, consequently, enhancing the expression of said gene. Alternatively, the activating transcription factor may bind an enhancer associated with said promoter, thereby promoting the activity of said promoter via said enhancer.

The term “lethal gene,” as used herein, refers to a gene whose expression product has a lethal effect, in sufficient quantity, on the organism within which the lethal gene is expressed.

The term “lethal effect,” as used herein, refers to a deleterious or sterilising effect, such as an effect capable of killing the organism per se or its offspring, or capable of reducing or destroying the function of certain tissues thereof, of which the reproductive tissues are particularly preferred, so that the organism or its offspring are sterile. Therefore, some lethal effects, such as poisons, will kill the organism or tissue in a short time-frame relative to their life-span, whilst others may simply reduce the organism's ability to function, for instance reproductively.

The term “tTAV gene variant,” as used herein, refers to a polynucleotide encoding the functional tTA protein but which differ in the sequence of nucleotides. These nucleotides may encode different tTA protein sequences, such as, for example, tTAV2 and tTAV3.

The term “promoter,” as used herein, refers to a DNA sequence, generally directly upstream to the coding sequence, required for basal and/or regulated transcription of a gene. In particular, a promoter is sufficient to allow initiation of transcription, generally having a transcription initiation start site and a binding site for the RNA polymerase transcription complex.

The term “minimal promoter,” as used herein, refers to a promoter as defined above, generally having a transcription initiation start site and a binding site for the polymerase complex, and further generally having sufficient additional sequence to permit these two to be effective. Other sequences, such as that which determines tissue specificity, for example, may be lacking.

The term “exogenous control factor,” as used herein, refers to a substance which is not found naturally in the host organism and which is not found in a host organism's natural habitat, or an environmental condition not found in a host organism's natural habitat. Thus, the presence of the exogenous control factor is controlled by the manipulator of a transformed host organism in order to control expression of the gene expression system.

The term “tetO element,” as used herein, refers to one or more tetO operator units positioned in series. The term, for example, “tetOx(number),” as used herein, refers to a tetO element consisting of the indicated number of tetO operator units. Thus, references to “tetOx7” indicates a tetO element consisting of seven tetO operator units. Similarly, references to “tetOx14” refers to a tetO element consisting of 14 tetO operator units, and so on.

The term “tra intron,” as used herein, refers to a splice control sequence wherein alternative splicing of the RNA transcript is regulated by the tra protein, for instance binding thereof, alone or in combination (i.e., when complexed) with TRA2.

Where reference to a particular nucleotide or protein sequence is made, it will be understood that this includes reference to any mutant or variant thereof, having substantially equivalent biological activity thereto. Preferably, the mutant or variant has at least 85%, preferably at least 90%, preferably at least 95%, preferably at least 99%, preferably at least 99.9%, and most preferably at least 99.99% sequence identity with the reference sequences.

However, it will be understood that despite the above sequence homology, certain elements, in particular the flanking nucleotides and splice branch site must be retained, for efficient functioning of the system. In other words, whilst portions may be deleted or otherwise altered, alternative splicing functionality or activity, to at least 30%, preferably 50%, preferably 70%, more preferably 90%, and most preferably 95% compared to the wild type should be retained. This could be increased compared to the wild type, as well, by suitably engineering the sites that bind alternative splicing factors or interact with the spliceosome, for instance.

As used herein, “splice control module” means a polynucleotide construct in that is incorporated into a vector that, when introduced into an insect, undergoes differential splicing (e.g., stage-specific, sex-specific, tissue-specific, germline-specific, etc.) and thus creates a different transcript in females than males if the splice control module confers differential splicing in a sex-specific manner.

As used herein, doublesex (dsx) refers to a gene in both male and female insects, such as Diptera that is subject to alternative splicing.

As used herein, “5′UTR,” refers to an untranslated region of an RNA transcript that is 5′ of the translated portion of the transcript and often contains a promoter sequence.

As used herein, “3′UTR,” refers to an untranslated region of an RNA transcript that is 3′ of the translated portion of the transcript and often contains a polyadenylation sequence.

### A. Overview of the Technology

The invention provides constructs and methods for differentially expressing proteins in insects in a sex-specific manner such that either a male insect or a female insect will express the protein and the other will not. The constructs of the invention have been engineered with a splice control module that is spliced differently in male insects than female insects. The splice control module may be operably linked to a heterologous protein-encoding polynucleotide such that the heterologous protein of interest is expressed in a sex-specific manner when introduced into an insect species. The constructs of the invention also may contain other elements for regulating expression in an insect, for identifying insects that have an integrated construct in their genome, and for selecting transformed cells, for example as will be described more fully below.

i. The Splice Control Module

Thus, in a first aspect, the present invention provides a splice control module polynucleotide sequence which provides for differential splicing (e.g., sex-specific, stage-specific, germline-specific, tissue-specific, etc.) in an organism. In particular, the invention provides a splice control module which provides for sufficient female-specificity of the expression of a gene of interest to be useful. In certain embodiments of the invention, the gene of interest is a gene that imparts a deleterious, lethal or sterilizing effect. For convenience, the description will refer to a lethal effect, however, it will be understood that the splice module may be used on other genes of interest as described in further detail below.

Expression of the dominant lethal genes of the transgene may be sex-specific, or be a combination of sex-specific and stage-specific, germline-specific or tissue-specific, due to the presence of at least one splice control module in each gene expression system operably linked to a gene of interest to be differentially expressed. In some embodiments, the sex-specific expression is female-specific. The splice control module in each gene expression sequence allows an additional level of control of protein expression, in addition to the promoter.

The gene of the splice control module comprises a coding sequence for a protein or polypeptide, i.e., at least one exon, and preferably two or more exons, capable of encoding a polypeptide, such as a protein or fragment thereof. Preferably, the different exons are differentially spliced together to provide alternative mRNAs. Preferably, said alternative spliced mRNAs have different coding potential, i.e., encode different proteins or polypeptide sequences. Thus, the expression of the coding sequence is regulated by alternative splicing.

Each splice control module in the system comprises at least one splice acceptor site and at least one splice donor site. The number of donor and acceptor sites may vary, depending on the number of segments of sequence that are to be spliced together.

In some embodiments, the splice control module regulates the alternative splicing by means of both intronic and exonic nucleotides. It will be understood that in alternative splicing, sequences may be intronic under some circumstances (i.e., in some alternative splicing variants where introns are spliced out), but exonic under other. In other embodiments, the splice control module is an intronic splice control module. In other words, it is preferred that said splice control sequence is substantially derived from polynucleotides that form part of an intron and are thus excised from the primary transcript by splicing, such that these nucleotides are not retained in the mature mRNA sequence.

As mentioned above, exonic sequences may be involved in the mediation of the control of alternative splicing, but it is preferred that at least some intronic control sequences are involved in the mediation of the alternative splicing.

The splice control module may be removed from the pre-RNA, by splicing or retained so as to encode a fusion protein of at least a portion of the gene of interest to be differentially expressed. Preferably, the splice control module does not result in a frameshift in the splice variant produced. Preferably, this is a splice variant encoding a full-length functional protein.

Interaction of the splice control module with cellular splicing machinery, e.g., the spliceosome, leads to or mediates the removal of a series of, preferably, at least 50 consecutive nucleotides from the primary transcript and ligation (splicing) together of nucleotide sequences that were not consecutive in the primary transcript (because they, or their complement if the antisense sequence is considered, were not consecutive in the original template sequence from which the primary transcript was transcribed). Said series of at least 50 consecutive nucleotides comprises an intron. This mediation acts preferably in a sex-specific, more preferably, female-specific, manner such that equivalent primary transcripts in different sexes, and optionally also in different stages, tissue types, etc., tend to remove introns of different size or sequence, or in some cases may remove an intron in one case but not another. This phenomenon, the removal of introns of different size or sequence in different circumstances, or the differential removal of introns of a given size or sequence, in different circumstances, is known as alternative splicing. Alternative splicing is a well-known phenomenon in nature, and many instances are known.

Where mediation of alternative splicing is sex-specific, it is preferred that the splice variant encoding a functional protein to be expressed in an organism is the F1 splice variant, i.e., a splice variant where the F denotes it is found only or predominantly in females, although this is not essential.

When exonic nucleotides are to be removed, then these must be removed in multiples of three (entire codons), if it is desired to avoid to avoid a frameshift, but as a single nucleotide or multiples of two (that are not also multiples of three) if it is desired to induce a frameshift. It will be appreciated that if only one or certain multiples of two nucleotides are removed, then this could lead to a completely different protein sequence being encoded at or around the splice junction of the mRNA.

This is particularly the case in an embodiment of the system where cassette exons are used to interrupt an open reading frame in some splice variants but not others, such as in, for example, tra, especially Cctra (see below).

Correspondingly for configurations where all or part of a functional open reading frame is on a cassette exon, it is preferred that this cassette exon is included in transcripts found only or predominantly in females, and preferably such transcripts are, individually or in combination, the most abundant variants found in females, although this is not essential.

In one preferred embodiment, sequences are included in a hybrid or recombinant sequence or construct which are derived from naturally occurring intronic sequences which are themselves subject to alternative splicing, in their native or original context. Therefore, an intronic sequence may be considered as one that forms part of an intron in at least one alternative splicing variant of the natural analogue. Thus, sequences corresponding to single contiguous stretches of naturally occurring intronic sequence are envisioned, but also hybrids of such sequences, including hybrids from two different naturally occurring intronic sequences, and also sequences with deletions or insertions relative to single contiguous stretches of naturally occurring intronic sequence, and hybrids thereof. Said sequences derived from naturally occurring intronic sequences may themselves be associated, in the invention, with sequences not themselves part of any naturally occurring intron. If such sequences are transcribed, and preferably retained in the mature RNA in at least one splice variant, they may then be considered exonic.

It will also be appreciated that reference to a “frame shift” could also refer to the direct coding of a stop codon, which is also likely to lead to a non-functioning protein as would a disruption of the spliced mRNA sequence caused by insertion or deletion of nucleotides. Production from different splice variants of two or more different proteins or polypeptide sequences of differential function is also envisioned, in addition to the production of two or more different proteins or polypeptide sequences of which one or more has no predicted or discernable function. Also envisioned is the production from different splice variants of two or more different proteins or polypeptide sequences of similar function, but differing subcellular location, stability or capacity to bind to or associate with other proteins or nucleic acids.

Preferred examples of this include a modified dsx intron. In this instance, it may be preferable to delete, as we have done in the Examples, sizable amounts from alternatively spliced introns, e.g., 90% or more of an intron in some cases, whilst still retaining the alternative splicing function. Thus, whilst large deletions are envisioned, it is also envisaged that smaller, e.g., even single nucleotide insertions, substitutions or deletions are also preferred.

ii. Examples of Splice Modules
a. Tra Sequences

As mentioned above, in some embodiments the manner or mechanism of alternative splicing is sex-specific, preferably female-specific, and any suitable splice control sequence may be used. In some embodiments, at least one splice control module is derived from a tra intron. The Ceratitis capitata tra intron from the transformer gene was initially characterised by Pane et al. (2002) Development 129:3715-3725. In insects, for instance, the tra protein is differentially expressed in different sexes. In particular, the tra protein is known to be present largely in females and, therefore, mediates alternative splicing in such a way that a coding sequence is expressed in a sex-specific manner, i.e., that in some cases a protein is expressed only in females or at a much higher level in females than in males or, alternatively, in other cases a protein is expressed only in males, or at a much higher level in males than in females. The mechanism for achieving this sex-specific alternative splicing mediated by the tra protein or the TRA/TRA-2 complex is known and is discussed, for instance, in Pane et al. (2002) Development 129:3715-3725.

It will be appreciated that homologues of the Ceratitis capitata tra intron from the transformer gene exist in other species, and these can be easily identified in said species and also in their various genera. Thus, when reference is made to tra it will be appreciated that this also relates to tra homologues in other species. Thus, in some embodiments each of the alternative splicing mechanisms is independently derived from the Ceratitis capitata tra intron (Cctra), or from another ortholog or homolog. In some embodiments, the ortholog or homologue is from an arthropod, such as an insect of the order Diptera, such as a tephritid. In other embodiments, the ortholog or homologue is from the genus Cochliomyia, Glossina, Lucilia, Musca, Ceratitis, Bactrocera, Anastrepha or Rhagoletis. In other embodiments, the ortholog or homolog is from Ceratitis rosa, or Bactrovera zonata. In further embodiments, the ortholog or homolog is from B. zonata, and this ortholog or homolog is referred to herein as Bztra (GenBank accession number BzTra 10397268). Orthologs may also be from the Order Hymenoptera, or Coleoptera. Examples, include, but are not limited to Apis cerana, Apis dorsata, Apis florea, Apis mellifera, Atta cephalotes, Bombus impatiens, Bombus terrestris, Camponotus floridanus, Euglossa hemichlora, Harpegnathos saltator, Linepithema humile, Melipona compressipes, Megachile rotundata, Nasonia giraulti, Nasonia longicornis, Nasonia vitripennis, Pogonomyrmex barbatus, Solenopsis invicta, and Tribolium castaneum.

The splicing pattern in Cctra in particular is well conserved, with those transcripts found in males containing additional exonic material relative to the F1 transcript, such that these transcripts do not encode full-length, functional tra protein. By contrast, the F1 transcript does encode full-length, functional tra protein; this transcript is substantially female-specific at most life-cycle stages, though it is speculated that very early embryos of both sexes may contain a small amount of this transcript. We describe the sequence spliced out of the F1 transcript, but not the male-specific or non-sex-specific transcripts, as the tra intron, or even the tra F1 intron. Thus the version of this sequence found in the Cctra gene is the Cctra intron.

Thus the tra gene is regulated in part by sex-specific alternative splicing, while its key product, the tra protein, is itself involved in alternative splicing. In insects, sex-specific alternative splicing mediated by the tra protein, or a complex comprising the tra and TRA2 proteins, include Dipteran splice control sequences derived from the doublesex (dsx) gene and also the tra intron itself, although this would exclude the tra intron from Drosophila (Dmtra), which is principally mediated by the Sxl gene product in Drosophila, rather than tra or the TRA/TRA2 complex. Outside of Drosophila, the Sxl gene product is not differentially expressed in the different sexes. Sxl is not thought to act in the mediation of sex-specific alternative splicing in non-Drosophilid insects.

By “derived” it will be understood that, using reference to the tra intron, this refers to sequences that approximate to or replicate exactly the tra intron, as described in the art, in this case by Pane et al. (2002), supra. However, it will be appreciated that, as these are intronic sequences, that some nucleotides can be added or deleted or substituted without a substantial loss in function.

If more than one splice control module is incorporated into a gene expression system of the invention, the splice control module may be the same or different. In some embodiments, it is preferred that the splice control modules are derived from different species in order to reduce the risk of recombination. Thus, in some embodiments, one of the first and second splice control sequences is Cctra and the other is derived from a different species. In one embodiment, one of the first and second splice control sequences is Cctra and the other is Bztra (GenBank accession number BzTra KJ397268).

In a particular embodiment, the first splice control sequence is Cctra and the second splice control sequence is Bztra (GenBank accession number BzTra KJ397268). The exact length of the splice control sequence derived from the Ira intron is not essential, provided that it is capable of mediating alternative splicing. In this regard, it is thought that around 55 to 60 nucleotides is the minimum length for a modified Ira intron, although the wild type Ira intron (F1 splice variant) from C. capitata is in the region of 1345 nucleotides long.

b. Actin-4

In other embodiments, at least one of the splice control sequences is derived from the alternative splicing mechanism of the Actin-4 gene derived from an arthropod, preferably a tephritid. In embodiments wherein more than one splice sequence is derived from Actin-4, they may be derived from the same or from different tephritid species. In some embodiments, each Actin-4 gene is independently derived from a species of the Ceratitis, the Bactrocera, the Anastrepha or the Rhagoletis genera. In other embodiments, the first and second Actin-4 genes are independently derived from Ceratitis capitata, Bactrocera oleae, Ceratitis rosa or Bactrocera zonata. In some embodiments, at least one of the first and second Actin-4 genes is derived from Ceratitis capitata. In embodiments wherein more than one splice control sequence is derived from Actin-4, the splice control sequences may be derived from the same species. However, it is preferred that the splice control sequences are derived from different species in order to reduce recombination.

c. Doublesex

In some embodiments, at least one of the splice control sequences comprises at least a fragment of the doublesex (dsx) gene derived from an arthropod, such as a tephritid. In some embodiments, more than one splice control sequence (e.g., both the first and second splice control sequences) is derived from dsx, and the dsx genes are derived from the same or different species. In some embodiments, the dsx gene is derived from a species of the Order Diptera, such as, but not limited to those of the genus Aedes, Anopheles, Cochliomyia, Culex, Drosophila, Glossina, Lucilia, Lutzomyia, Ceratitis, Bactrocera, Anastrepha, Mayetiola, Megaselia, Musca, Phlebotomus and Rhagoletis. In some embodiments, the dsx genes are independently derived from Aedes aegypti, Anopheles spp., Anopheles gambiae, Anastrepha spp., Ceratitis capitata, Bactrocera oleae, Bactrocera dorsalis, Bactrocera zonata, Bactrocera correcta, Bactrocera tryoni, Ceratitis rosa, Cochliomyia homnivorax, Cochliomyia macellaria, Culex quinquefasciatus, Drosophila Americana, Drosophila erecta, Drosophila hydei, Drosophila mauritania, Drosophila melanogaster, Drosophila sechellia, Drosophila simulans, Drosophila virilis, Glossina morsitans, Lucilia cuprina, Lucilia sericata, Lutzomyia longipalpis, Mayetiola destructor, Megaselia scalaris, Musca domestica, and Phlebotomus papatasi.

In some embodiments, the dsx gene is derived from a species of the Order Phtiraptera, such as, for example, pediculus humanus corporis.

In some embodiments, the dsx gene is derived from a species of the Order Hemiptera, including such species as, but not limited to Acyrthosiphon pisum and Rhodnius prolixus.

In some embodiments, the dsx gene is derived from a species of the Order Hymenoptera, including, but not limited to insects of the genera Apis, Atta, Bombus, Camponotus, Euglossa, Harpegnathos, Linepithema, Melipona, Megachile, Nasonia, Pogonomyrmex, and Solenopsis. Examples of suitable species, include, but are not limited to Apis cerana, Apis dorsata, Apis florea, Apis mellifera, Atta cephalotes, Bombus impatiens, Bombus terrestris, Camponotus floridanus, Euglossa hemichlora, Harpegnathos saltator, Linepithema humile, Melipona compressipes, Megachile rotundata, Nasonia giraulti, Nasonia longicornis, Nasonia vitripennis, Pogonomyrmex barbatus, Solenopsis invicta.

In some embodiments, the dsx gene is derived from a species of the Order Lepidoptera, including but not limited to insects of the genera Antheraea, Bombyx, Danaus, Heliconius, and Ostrinia. Examples of suitable species, include, but are not limited to Antheraea assama, Antheraea mylitta, Bombyx mori, Danaus plexippus, Heliconius Melpomene, Plutella xylostella, Pectinophora gossypiella and Ostrinia scapulalis.

In some embodiments, the dsx gene is derived from a species of the Order Coleoptera, including, but not limited to insects of the genera Dendroctonus, Onthophagus, and Tribolium. Examples of suitable species, include, but are not limited to Dendroctonus ponderosae, Onthophagus sagittarius, Onthophagus taurus, and Tribolium castaneum.

In some embodiments, the dsx gene is derived from a species of the Order Strepsiptera, including, but not limited to insects of the genus Mengenilla (e.g., Mengenilla moldrzyki).

In some embodiments, at least one of the first and second dsx genes is derived from the same insect, such as, for example, Aedes aegypti. In embodiments wherein more than one splice control sequence is derived from dsx, the splice control sequences may be derived from the same species. In other embodiments, the splice control sequences are derived from different species.

In one embodiment, the present invention provides a doublesex (dsx) splice control module polynucleotide wherein the splice control module comprises (from 5′ to 3′): at least a portion of an exon 4 of dsx, preferably the entire exon (an example is shown for Aedes aegypti as SEQ ID NO:13), a truncated intron 4 of dsx comprising at least a 5′ terminal fragment of the dsx intron 4 that contains at least a portion of the 5′ end of intron 4 (an example is shown for Aedes aegypti as SEQ ID NO:12) and a 3′ fragment of the dsx intron 4 that contains at least a portion of the 3′ end of intron 4 (an example is shown for Aedes aegypti as SEQ ID NO:11), at least a portion of an exon 5a of dsx, preferably the entire exon 5a (an example is shown for Aedes aegypti as SEQ ID NO:6), at least a portion of an intron 5 of dsx, preferably the entire intron 5 (an example is shown for Aedes aegypti as SEQ ID NO:10), a modified exon 5b of dsx (an example is shown for Aedes aegypti as SEQ ID NO:7), a truncated intron 6 that contains at least a portion of the 5′ end of intron 6 of dsx (an example is shown for Aedes aegypti as SEQ ID NO:9) linked to at least a portion of the 3′ fragment of intron 6 of dsx (an example is shown for Aedes aegypti as SEQ ID NO:8) forming a truncated intron 6, and at least a portion of the 5′ region of an exon 6 of dsx (an example is shown for Aedes aegypti as SEQ ID NO:5).

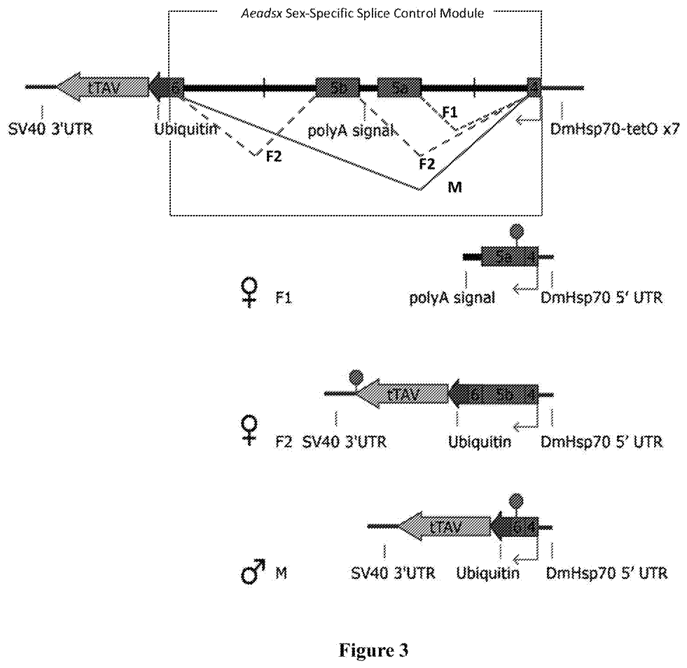

The dsx splice control module allows a sex-specific splicing of the module to a polypeptide encoding polynucleotide such that the polypeptide is expressed in a sex-specific manner. In a specific example, two principle transcripts are made in female Aedes: Transcript F1 contains exon 4, 5a, and intron 5, which acts as a 3′ UTR and contains a polyadenylation signal; Transcript F2 contains exon 4, exon 5b and truncated exon 6 together with the heterologous gene of interest in frame with the rest of the transcript and with the translation start site 5′ to exon 4. In the male Aedes, the splice form contains exon 4 and exon 6, but the heterologous gene of interest is out of frame with the translation start site 5′ to exon 4 (FIG. 3).

While in some embodiments it is envisaged that the splice control modules are derived from the same gene or intron of origin, in other embodiments the splice control modules are derived from different genes or introns of origin. For example, in some embodiments, one of the splice control modules is derived from the tra intron and the other splice control module is derived from the Actin-4 gene or the dsx gene.

Preferably, the splice control module is 3′ to the start codon. Where the splice control module is 3′ to the start codon, it is preferred that it is also 5′ to the first in-frame stop codon (that is 3′ to and in frame with the start codon), so that alternative splicing yields transcripts that encode different protein or polypeptide sequences. Thus in a preferred embodiment, the construct or polynucleotide sequence comprises the following elements in 5′ to 3′ order, with respect to the sense strand or primary transcript: transcription start, translation start, intron capable of alternative splicing, coding sequence for all or part of a protein, stop codon.

iii. Splicing

Introns typically consist of the following features (given here as the sense DNA sequence 5′ to 3′); in RNA thymine (T) will be replaced by uracil (U)):
a. 5′ end (known as the splice “donor”): GT (or possibly GC)
b. 3′ end (known as the splice “acceptor”): AG
c. Upstream/5′ of the acceptor (known as the “branch point”): A-polypyrimidine tract, i.e. AYYYYY . . . Yn

The terminal nucleotides of exons immediately adjacent to the 5′ intronic splice “donor” and the 3′ intronic splice “acceptor” are typically G.

In some embodiments, the splice control module is immediately adjacent, in the 3′ direction, the start codon, so that the G of the ATG is 5′ to the start (5′ end) of the splice control module. This may be advantageous as it allows the G of the ATG start codon to be the 5′ G flanking sequence to the splice control module.

Alternatively, the splice control module is 3′ to the start codon but within 10,000 exonic bp, 9,000 exonic bp, 8,000 exonic bp, 7,000 exonic bp, 6,000 exonic bp, 5,000 exonic bp, 4,000 exonic bp, exonic 3,000 bp, exonic 2000, bp, or 1000 exonic bp, preferably 500 exonic bp, preferably 300 exonic bp, preferably 200 exonic bp, preferably 150 exonic bp, preferably 100 exonic bp, more preferably 75 exonic bp, more preferably 50 exonic bp, more preferably 30 exonic bp, more preferably 20 exonic bp, and most preferably 10 or even 5, 4, 3, 2, or 1 exonic bp.

Preferably, branch points are included in each splice control sequence, as described above. A branch point is the sequence to which the splice donor is initially joined which shows that splicing occurs in two stages, in which the 5′ exon is separated and then is joined to the 3′ exon.

The sequences provided can tolerate some sequence variation and still splice correctly. There are a few nucleotides known to be important. These are the ones required for all splicing. The initial GU and the final AG of the intron are particularly important and therefore preferred, as discussed elsewhere, though ˜5% of introns start GC instead. This consensus sequence is preferred, although it applies to all splicing, not specifically to alternative splicing.

iv. Heterologous Genes of Interest

The system is capable of expressing at least one protein of interest, i.e., said functional protein to be expressed in an organism. Said at least one protein of interest may have a therapeutic effect or may, be a marker (for instance DsRed, Green Fluorescent Protein (GFP) or one or more of their mutants or variants), or other markers that are well known in the art such as drug resistance genes. Other proteins of interest may be, for example, proteins that have a deleterious, lethal or sterilizing effect. Alternatively, a gene of interest may encode an RNA molecule that has an inhibitory effect. Further proteins to be expressed in the organism are, or course envisaged, in combination with said functional protein, preferably a lethal gene as discussed below.

It is preferred that the expression of the heterologous polynucleotide sequence leads to a phenotypic consequence in the organism. In some embodiments, the functional protein is not beta-galactosidase, but can be associated with visible markers (including fluorescence), viability, fertility, fecundity, fitness, flight ability, vision, and behavioural differences. It will be appreciated, of course, that, in some embodiments, the expression systems are typically conditional, with the phenotype being expressed only under some, for instance restrictive, conditions.

The at least one heterologous polynucleotide sequence to be expressed in an organism is a heterologous sequence. By “heterologous,” it would be understood that this refers to a sequence that would not, in the wild type, be normally found in association with, or linked to, at least one element or component of the at least one splice control sequence. For example, where the splice control sequence is derived from a particular organism, and the heterologous polynucleotide is a coding sequence for a protein or polypeptide, i.e., is a polynucleotide sequence encoding a functional protein, then the coding sequence could be derived, in part or in whole, from a gene from the same organism, provided that that the origin of at least some part of the transcribed polynucleotide sequence was not the same as the origin of the at least one splice control sequence. Alternatively, the coding sequence could be from a different organism and, in this context, could be thought of as “exogenous”. The heterologous polynucleotide could also be thought of as “recombinant,” in that the coding sequence for a protein or polypeptide are derived from different locations, either within the same genome (i.e., the genome of a single species or sub-species) or from different genomes genomes from different species or subspecies), or synthetic sources.

Heterologous can refer to a sequence other than the splice control sequence and can, therefore, relate to the fact the promoter, and other sequences such as 5′ UTR and/or 3′UTR can be heterologous to the polynucleotide sequence to be expressed in the organism, provided that said polynucleotide sequence is not found in association or operably linked to the promoter, 5′ UTR and/or 3′UTR, in the wild type, i.e., the natural context of said polynucleotide sequence, if any.

It will be understood that heterologous also applies to “designer” or hybrid sequences that are not derived from a particular organism but are based on a number of components from different organisms, as this would also satisfy the requirement that the sequence and at least one component of the splice control sequence are not linked or found in association in the wild type, even if one part or element of the hybrid sequence is so found, as long as at least one part or element is not. It will also be understood that synthetic versions of naturally occurring sequences are envisioned. Such synthetic sequences are also considered as heterologous, unless they are of identical sequence to a sequence which would, in the wild type or natural context, be normally found in association with, or linked to, at least one element or component of the at least one splice control sequence.

This applies equally to where the heterologous polynucleotide is a polynucleotide for interference RNA.

In one embodiment, where the polynucleotide sequence to be expressed comprises a coding sequence for a protein or polypeptide, it will be understood that reference to expression in an organism refers to the provision of one or more transcribed RNA sequences, preferably mature mRNAs, but this may, preferably, also refer to translated polypeptides in said organism.

a. Lethal Genes

In some embodiments, the functional protein to be expressed in an organism has a lethal, deleterious or sterilizing effect. Where reference is made herein to a lethal effect, it will be appreciated that this extends to a deleterious or sterilizing effect, such as an effect capable of killing the organism per se or its offspring, or capable of reducing or destroying the function of certain tissues thereof, of which the reproductive tissues are particularly preferred, so that the organism or its offspring are sterile. Therefore, some lethal effects, such as poisons, will kill the organism or tissue in a short time-frame relative to their life-span, whilst others may simply reduce the organism's ability to function, for instance reproductively.

A lethal effect resulting in sterilization is particularly preferred, as this allows the organism to compete in the natural environment (“in the wild”) with wild-type organisms, but the sterile insect cannot then produce viable offspring. In this way, the present invention achieve a similar or better result to techniques such as the Sterile Insect Technique (SIT) in insects, without the problems associated with SIT, such as the cost, danger to the user, and reduced competitiveness of the irradiated organism.

Preferably, the system comprises at least one positive feedback mechanism, namely at least one functional protein to be differentially expressed, via alternative splicing, and at least one promoter therefor, wherein a product of a gene to be expressed serves as a positive transcriptional control factor for the at least one promoter, and whereby the product, or the expression of the product, is controllable. Preferably, an enhancer is associated with the promoter, the gene product serving to enhance activity of the promoter via the enhancer.

The present invention allows for selective control of the expression of the first and/or second dominant lethal genes, thereby providing selective control of the expression of a lethal phenotype. It will therefore be appreciated that each of the lethal genes encodes a functional protein, such as described in WO2005/012534.

Each of the lethal genes has a lethal effect which is conditional. An example of suitable conditions includes temperature, so that the lethal is expressed at one temperature but not, or to a lesser degree, at another temperature. Another example of a suitable condition is the presence or absence of a substance, whereby the lethal is expressed in either the presence or absence of the substance, but not both. It is preferred that the effect of the lethal gene is conditional and is not expressed under permissive conditions requiring the presence of a substance which is absent from the natural environment of the organism, such that the lethal effect of the lethal system occurs in the natural environment of the organism.

Each lethal genetic system may act on specific cells or tissues or impose its effect on the whole organism. Systems that are not strictly lethal but impose a substantial fitness cost are also envisioned, for example leading to blindness, flightlessness (for organisms that could normally fly), or sterility. Systems that interfere with sex determination are also envisioned, for example transforming or tending to transform all or part of an organism from one sexual type to another.

In some embodiments, the product of at least one of the lethal genes is preferably an apoptosis-inducing factor, such as the AIF protein described for instance in Candé et al. (2002) J. Cell Science 115:4727-4734) or homologues thereof. AIF homologues are found in mammals and even in invertebrates, including insects, nematodes, fungi, and plants, meaning that the AIF gene has been conserved throughout the eukaryotic kingdom. In other embodiments, the product of at least one of the lethal genes is Hid, the protein product of the head involution defective gene of Drosophila melanogaster, or Reaper (Rpr), the product of the reaper gene of Drosophila, or mutants thereof. Use of Hid was described by Heinrich and Scott (2000) Proc. Natl Acad. Sci USA 97:8229-8232). Use of a mutant derivative, HidAla5 was described by Horn and Wimmer (2003) Nature Biotechnology 21:64-70). Use of a mutant derivative of Rpr, RprKR, is described herein (see also White et al. (1996); Science 271(5250):805-807; Wing et al. (2001) Mech. Dev. 102(1-2):193-203; and Olson et al. (2003) J. Biol. Chem. 278(45):44758-44768. Both Rpr and Hid are pro-apoptotic proteins, thought to bind to IAP1. IAP1 is a well-conserved anti-apoptotic protein. Hid and Rpr are therefore expected to work across a wide phylogenetic range (Huang et al. (2002); Vemooy et al. (2000) J. Cell Biol. 150(2):F69-76) even though their own sequence is not well conserved.

Nipp1Dm, the Drosophila homologue of mammalian Nipp1 (Parker et al. (2002) Biochemical Journal 368:789-797; Bennett et al., (2003) Genetics 164:235-245) are utilized in some embodiments. Nipp1Dm is another example of a protein with lethal effect if expressed at a suitable level, as would be understood by the skilled person. Indeed, many other examples of proteins with a lethal effect will be known to the person skilled in the art.

In other embodiments, the lethal genes is tTA or a tTAV gene variant, where tTA denotes ‘tetracycline repressible Trans-Activator’ and V denotes ‘Variant.’ tTAV is an analogue of tTA, wherein the sequence of tTA has been modified to enhance the compatibility with the desired insect species. Variants of tTAV are possible, encoding the tTA protein, such that the tTAV gene products have the same functionality as the tTA gene product. Thus, the variants of the tTAV gene comprise modified nucleotide sequences as compared to the tTA nucleotide sequence and to each other, but encode proteins with the same function. Thus, tTAV gene variants can be used in the place of tTA. In some embodiments the tTA Variant proteins contain amino acid substitutions, additions or deletions. Any combination of lethal genes may be used, and, in some embodiments, the lethal genes are the same while, in other embodiments, the lethal genes are different. The improved penetrance of the lethal effect and the earlier onset of lethality is achieved by an accumulation of lethal product.

In particular embodiments, each of the first and second lethal genes is independently tTA or a tTAV gene variant. In some embodiments, each of the first and second lethal gene is independently one of tTAV (SEQ ID NO:3), tTAV2 (SEQ ID NO:27) and tTAV3 (SEQ ID NO:28). In other embodiments, the first and second lethal genes are the same. In further embodiments, one of the first and second lethal genes is tTAV (SEQ ID NO:3) and the other gene is tTAV3 (SEQ ID NO:28). However, any combination of tTAV variants may be used; thus, in some embodiments, one of the first and second genes is tTAV (SEQ ID NO:3) and the other is tTAV2 (SEQ ID NO:27), while, in a further embodiment, one of the first and second genes is tTAV2 (SEQ ID NO:27) and the other gene is tTAV3 (SEQ ID NO:28). In other embodiments, the first lethal gene is tTAV (SEQ ID NO:3) and the second lethal gene is tTAV3 (SEQ ID NO:28).

b. RNAi

The polynucleotide sequence to be expressed may comprise polynucleotides for interference RNA (RNAi). In some embodiments, where the polynucleotide sequence to be expressed comprises polynucleotides for interference RNA, it will also be understood that reference to expression in an organism refers to the interaction of the polynucleotides for interference RNA, or transcripts thereof, in the RNAi pathway, for instance by binding of Dicer (RNA Pol III-like enzyme) or formation of small interfering RNA (siRNA). Such sequences are capable of providing, for instance, one or more stretches of double-stranded RNA (dsRNA), preferably in the form of a primary transcript, which in turn is capable of processing by the Dicer. Such stretches include, for instance, stretches of single-stranded RNA that can form loops, such as those found in short-hairpin RNA (shRNA), or with longer regions that are substantially self-complementary.

Indeed, it is particularly preferred that the polynucleotides for interference RNA comprise siRNA sequences and are, therefore, preferably 20-25 nucleotides long, especially where the organism is mammalian.

In insects and nematodes especially, it is preferred to provide portion of dsRNA, for instance by hairpin formation, which can then be processed by the Dicer system. Mammalian cells generally produce an interferon response against long dsRNA sequences, so for mammalian cells it is more common to provide shorter sequences, such as siRNAs. Antisense sequences or sequences having homology to microRNAs that are naturally occurring RNA molecules targeting protein 3′ UTRs are also envisaged as sequences for RNAi according to an embodiment of the present invention.

Thus, where the system is DNA, the polynucleotides for interference RNA are deoxyribonucleotides that, when transcribed into pre-RNA ribonucleotides, provide a stretch of dsRNA, as discussed above.

Polynucleotides for interference RNA are particularly preferred when said polynucleotides are positioned to minimise interference with alternative splicing. This may be achieved by distal positioning of these polynucleotides from the alternative splice control sequences, preferably 3′ to the control sequences. In another preferred embodiment, substantially self-complementary regions may be separated from each other by one or more splice control sequences, such as an intron, that mediate alternative splicing. Preferably, the self-complementary regions are arranged as a series of two or more inverted repeats, each inverted repeat separated by splice control sequence, preferably an intron, as defined elsewhere.

In this configuration, different alternatively spliced transcripts may have their substantially self-complementary regions separated by different lengths of non-self-complementary sequence in the mature (post-alternative-splicing) transcript. It will be appreciated that regions that are substantially self-complementary are those that are capable of forming hairpins, for instance, as portions of the sequence are capable of base-pairing with other portions of the sequence. These two portions do not have to be exactly complementary to each other, as there can be some mismatching or toleration of stretches in each portion that do not base-pair with each other. Such stretches may not have an equivalent in the other portion, such that symmetry is lost and “bulges” form, as is known with base-pair complementation in general.

In another preferred embodiment, one or more segment of sequence substantially complementary to another section of the primary transcript is positioned, relative to the at least one splice control sequence, so that it is not included in all of the transcripts produced by alternative splicing of the primary transcript. By this method, some transcripts are produced that tend to produce dsRNA while others do not; by mediation of the alternative splicing, e.g., sex-specific mediation, stage-specific mediation, germline-specific mediation, tissue-specific mediation, and combinations thereof, dsRNA may be produced in a sex-specific, stage-specific, germline-specific or tissue-specific manner, or combinations thereof

v. Fusion Leaders

In some embodiments it will be desirable to have the functional protein of interest free of the Splice Control Module protein sequence. In some embodiments, the Splice Control Module is operatively linked to a polypeptide-encoding polynucleotide that stimulates proteolytic cleave of a translated polypeptide (“Fusion Leader Sequences” for the polynucleotide and “Fusion Leader Polypeptide” for the encoded polypeptide). An example of such a Fusion Leader Sequence is ubiquitin encoding polynucleotide. Such a Fusion Leader Sequence may be operatively linked in frame to the 3′ end of the Splice Control Module and operatively linked in frame to the protein encoding gene of interest (i.e., from 5′ to 3′: Splice Control Module-Fusion Leader Sequence-Gene of interest). In such a case, the Splice Control Module/Fusion Leader Polypeptide is cleaved from the protein of interest by specific proteases in the cell. Aside from ubiquitin, any other similar fusion may be made in place of ubiquitin that would have the effect of stimulating a cleavage of the N-terminal Splice Control Module.

vi. Promoters and 5′UTRs

Each lethal gene is operably linked to a promoter, wherein said promoter is capable of being activated by an activating transcription factor or trans-activating encoded by a gene also included in at least one of the gene expression systems. It is preferred that any combination of promoter and Splice Control Module is envisaged. The promoter is preferably specific to a particular protein having a short temporal or confined spatial effect, for example a cell-autonomous effect.

The promoter may be a large or complex promoter, but these often suffer the disadvantage of being poorly or patchily utilised when introduced into non-host insects. Accordingly, in some embodiments, it is preferred to employ minimal promoters. It will be appreciated that minimal promoters may be obtained directly from known sources of promoters, or derived from larger naturally occurring, or otherwise known, promoters. Suitable minimal promoters and how to obtain them will be readily apparent to those skilled in the art. For example, suitable minimal promoters include a minimal promoter derived from Hsp70, a P minimal promoter, a CMV minimal promoter, an Acf5C-based minimal promoter, a BmA3 promoter fragment, and an Adh core promoter (Bieschke, E. et al. (1998) Mol. Gen. Genet., 258:571-579). Not all minimal promoters will necessarily work in all species of insect, but it is readily apparent to those skilled in the art as to how to ensure that the promoter is active. It is preferred that at least one of the operably-linked promoters present in the invention is active during early development of the host organism, and particularly preferably during embryonic stages, in order to ensure that the lethal gene is expressed during early development of the organism.

In some embodiments, the promoter can be activated by environmental conditions, for instance the presence or absence of a particular factor such as tetracycline in the tet system described herein, such that the expression of the gene of interest can be easily manipulated by the skilled person. Alternatively, a preferred example of a suitable promoter is the hsp70 heat shock promoter, allowing the user to control expression by variation of the environmental temperature to which the hosts are exposed in a lab or in the field, for instance. Another preferred example of temperature control is described in Fryxell and Miller (1995) J. Econ. Entomol. 88:1221-1232.

In some embodiments, the promoter is the sryα embryo-specific promoter (Horn and Wimmer (2003) Nat. Biotechnol. 21(1):64-70) from Drosophila melanogaster, or its homologues, or promoters from other embryo-specific or embryo-active genes, such as that of the Drosophila gene slow as molasses (slam), or its homologues from other species.

Alternatively, the promoter may be specific for a broader class of proteins or a specific protein that has a long-term and/or wide system effect, such as a hormone, positive or negative growth factor, morphogen or other secreted or cell-surface signaling molecule. This would allow, for instance, a broader expression pattern so that a combination of a morphogen promoter with a stage-specific alternative splicing mechanism could result in the morphogen being expressed only once a certain life-cycle stage was reached, but the effect of the morphogen would still be felt (i.e., the morphogen can still act and have an effect) beyond that life-cycle stage. Preferred examples would be the morphogen/signaling molecules Hedgehog, Wingless/WNTs, TGFβ/BMPs, EGF and their homologues, which are well-known evolutionarily-conserved signaling molecules.

It is also envisaged that a promoter that is activated by a range of protein factors, for instance transactivators, or which has a broad systemic effect, such as a hormone or morphogen, could be used in combination with an alternative splicing mechanism to achieve a tissue and sex-specific control or sex and stage-specific control, or other combinations of stage-, tissue, germ-line- and sex-specific control.

It is also envisaged that more than one promoter, and optionally an enhancer therefor, can be used in the present system, either as alternative means for initiating transcription of the same protein or by virtue of the fact that the genetic system comprises more than one gene expression system (i.e., more than one gene and its accompanying promoter).

In some embodiments, at least one of the promoters is the minimal promoter is a heat shock promoter, such as Hsp70. In other embodiments, at least one of the promoters is the sryα embryo-specific promoter (Horn and Wimmer (2003) Nat. Biotechnol. 21(1):64-70) from Drosophila melanogaster, or its homologues, or promoters from other embryo-specific or embryo-active genes, such as that of the Drosophila gene slow as molasses (slam), or its homologues from other species.

In some embodiments, at least one of the promoters is a minimal promoter. In some embodiments, each of the promoters is independently Baculovirus Autographica californica nucleopolyhedrosisvirus (AcNPV) promoter IE1, Hsp70, Hsp73 or sryα. In preferred embodiments, one of the first and second promoters is Hsp70 and the other is sryα. In one embodiment, the first promoter is Hsp70 and the second promoter is sryα. Each gene expression system further comprises a gene encoding an activating transcription factor, wherein each activating transcription factor activates the expression of a lethal gene of the transgene. Thus, each gene encoding an activating transcription factor is able to be expressed by the host organism, to produce a functional protein. In particular, each activating transcription factor is capable of activating at least one promoter, wherein the promoter is operably linked to a lethal gene. Consequently, when an activating transcription factor activates a promoter, the expression of the lethal gene operably linked to the promoter is up-regulated. Each activating transcription factor may act on either the first or the second promoter, or each activating transcription factor may act on both the first and the second promoter. It is preferred that, when more than one activating transcription factor is expressed, more than one promoter is activated. Thus, when both the first and the second activating transcription factors are expressed, both the first and the second promoters are activated. The gene products serving as activating transcription factors may act in any suitable manner. For example, the activating transcription factors may bind to an enhancer located in proximity to the at least one promoter, thereby serving to enhance polymerase binding at the promoter. Other mechanisms may be employed, such as repressor countering mechanisms, such as the blocking of an inhibitor of transcription or translation. Transcription inhibitors may be blocked, for example, by the use of hairpin RNA's or ribozymes to block translation of the mRNA encoding the inhibitor, or the product may bind the inhibitor directly, thereby preventing inhibition of transcription or translation.

vii. Repressible Elements

Preferably, the polynucleotide expression system is a recombinant dominant lethal genetic system, the lethal effect of which is conditional. Suitable conditions include temperature, so that the system is expressed at one temperature but not, or to a lesser degree, at another temperature, for example. The lethal genetic system may act on specific cells or tissues or impose its effect on the whole organism. It will be understood that all such systems and consequences are encompassed by the term lethal as used herein. Similarly, “killing”, and similar terms refer to the effective expression of the lethal system and thereby the imposition of a deleterious or sex-distorting phenotype, for example death.

More preferably, the polynucleotide expression system is a recombinant dominant lethal genetic system, the lethal effect of which is conditional and is not expressed under permissive conditions requiring the presence of a substance which is absent from the natural environment of the organism, such that the lethal effect of the lethal system occurs in the natural environment of the organism.

In some embodiments, the coding sequences encode a lethal linked to a system such as the tet system described in WO 01/39599 and/or WO2005/012534.

Indeed it is preferred that the expression of said lethal gene is under the control of a repressible transactivator protein. It is also preferred that the gene whose expression is regulated by alternative splicing encode a transactivator protein such as tTA. This is not incompatible with the regulated protein being a lethal. Indeed, it is particularly preferred that it is both. In this regard, we particularly prefer that the system includes a positive feedback system as taught in WO2005/012534.

Preferably, the lethal effect of the dominant lethal system is conditionally suppressible.

Thus, in some embodiments wherein one or more of the dominant, lethal genes is tTA or a tTAV gene variant, an enhancer is a tetO element, comprising one or more tetO operator units. Upstream of a promoter, in either orientation, tetO is capable of enhancing levels of transcription from a promoter in close proximity thereto, when bound by the product of the tTA gene or a tTAV gene variant. In some embodiments, each enhancer is independently one of tetOx1, tetOx2, tetOx3, tetOx4, tetOx5, tetOx6, tetOx7, tetOx8, tetOx9, tetOx10, tetOx11, tetOx12, tetOx13, tetOx14, tetOx15, tetOx16, tetOx17, tetOx18, tetOx19, tetOx20 and tetOx21. In some embodiments, each enhancer is independently one of tetOx1, tetOx14 and tetOx21. In embodiments comprising more than one enhancer, the first enhancer is the same as or different from the second enhancer. An example of the TetOx7 element is shown in SEQ ID NO:14.

viii. Other Elements

In some embodiments, the system comprises other upstream, 5′ factors and/or downstream 3′ factors for controlling expression. Examples include enhancers such as the fat-body enhancers from the Drosophila yolk protein genes, and the homology region (hr) enhancers from baculoviruses, for example AcNPV Hr5. It will also be appreciated that the RNA products will include suitable 5′ and 3′ UTRs, for instance.

It will be understood that reference is made to start and stop codons between which the polynucleotide sequence to be expressed in an organism is defined, but that this does not exclude positioning of the at least one splice control sequence, elements thereof, or other sequences, such as introns, in this region. In fact, it will be apparent form the present description that the splice control sequence, can, in some embodiments, be positioned in this region.

Furthermore, the splice control sequence, for instance, can overlap with the start codon at least, in the sense that the G of the ATG can be, in some embodiments, be the initial 5′ G of the splice control sequence. Thus, the term “between” can be thought of as referring to from the beginning (3′ to the initial nucleotide, i.e., A) of the start codon, preferably 3′ to the second nucleotide of the start codon (i.e., T), up to the 5′ side of the first nucleotide of the stop codon. Alternatively, as will be apparent by a simple reading of a polynucleotide sequence, the stop codon may also be included.

ix. Vectors in General and Incorporate Elements Permitting Replication

In embodiments of the invention, the system is or comprises a plasmid. As mentioned above, this can be either DNA, RNA or a mixture of both. If the system comprises RNA, then it may be preferable to reverse-translate the RNA into DNA by means of a Reverse Transcriptase. If reverse transcription is required, then the system may also comprise a coding sequence for the RT protein and a suitable promoter therefor. Alternatively, the RTase and promoter therefore may be provided on a separate system, such as a virus. In this case, the system would only be activated following infection with that virus. The need to include suitable cis-acting sequences for the reverse transcriptase or RNA-dependent RNA polymerase would be apparent to the person skilled in the art.

However, it is particularly preferred that the system is predominantly DNA and more preferably consists only of DNA, at least with respect to the sequences to be expressed in the organism.

B. Introduction of Constructs into Organisms

Methods of introduction or transformation of the gene system constructs and induction of expression are well known in the art with respect to the relevant organism. It will be appreciated that the system or construct is preferably administered as a plasmid, but generally tested after integrating into the genome. Administration can be by known methods in the art, such as parenterally, intra-venous intra-muscularly, orally, transdermally, delivered across a mucous membrane, and so forth. Injection into embryos is particularly preferred. The plasmid may be linearised before or during administration, and not all of the plasmid may be integrated into the genome. Where only part of the plasmid is integrated into the genome, it is preferred that this part include the at least one splice control module capable of mediating alternative splicing.

Plasmid vectors may be introduced into the desired host cells by methods known in the art, such as, for example by transfection, electroporation, microinjection, transduction, cell fusion, DEAE dextran, calcium phosphate precipitation, lipofection (lysosome fusion), use of a gene gun, or a DNA vector transporter (see, e.g., Wu et al., (1992) J. Biol. Chem. 267:963; Wu et al. (1988) J. Biol. Chem. 263:14621; and Canadian Patent Application No. 2,012,311 to Hartmut et al.). The plasmid vector may be integrated into the host chromosome by any means known. Well-known methods of locus-specific insertion may be used, including, homologous recombination and recombinase-mediated genome insertion. In another embodiment, locus-specific insertion may be carried out by recombinase-site specific gene insertion. In one example piggyBac sequences may be incorporated into the vector to drive insertion of the vector into the host cell chromosome. Other technologies such as CRISPRs, TALENs, AttP/AttB recombination may also be employed.

### C. Genetically Engineered Insects

Suitable organisms under which the present system can be used include non-human mammals such as mice, rats and farm animals. Also preferred are fish, such as salmon and trout. Plants are also preferred, but it is particularly preferred that the host organism is an insect, preferably a Dipteran or tephritid.

The vectors of the invention may be used to create transgenic insects in a wide variety of genera and species. The insects that may be transformed with a vector of the invention include, but are not limited to those in the Order Diptera, especially higher Diptera, such as, for example, a tephritid fruit fly, such as Medfly (Ceratitis capitata), Mexfly (Anastrepha ludens), Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata) Caribbean fruit fly (Anastrepha suspensa) or West Indian fruit fly (Anastrepha obliqua). It is also particularly preferred that the host organism is a mosquito, preferably from the genera Stegomyia, Aedes, Anopheles or Culex. Particularly preferred are Stegomyia aegyptae, also known as Aedes aegypti, Stegornyia albopicta (also known as Aedes albopictus), Anopheles stephensi, Anopheles albimanus and Anopheles gambiae.

Within Diptera, another group which may be modified using a vector of the invention is Calliphoridae, such as, for example the New world screwworm (Cochliomyia hominivorax), and Old world screwworm (Chrysomya bezziana). Other dipteran species include Australian sheep blowfly (Lucilia cuprina), Agrornyza frontella (alfalfa blotch leafminer), Agromyza spp. (leaf miner flies), Chrysops spp. (deer flies), Contarinia spp. (Gall midges), Dasineura spp. (gall midges), Dasineura brassicae (cabbage gall midge), Delia spp., Delia platura (seedcorn maggot), Drosophila spp. (vinegar flies), Fannia spp. (filth flies), Fannia canicularis (little house fly), Fannia scalaris (latrine fly), Gasterophilus intestinalis (horse bot fly), Gracillia perseae, Haematobia irritans (horn fly), Hylemyia spp. (root maggots), Hypoderma lineation (common cattle grub), Liriomyza spp. (leafminer flies), Liriomyza brassica (serpentine leafminer), Melophagus ovinus (sheep ked), Musca spp. (muscid flies), Musca autumnalis (face fly), Musca domestica (house fly), Oestrus ovis (sheep bot fly), Oscinella frit (grass fly), Pegomyia betae (beet leafminer), Phorbia spp., Psila rosae (carrot rust fly), Rhagoletis pomonella (apple maggot), Sitodiplosis mosellana (orange wheat blossom midge), Stomoxys calcarans (stable fly), Tabanus spp. (horse flies) and Tipula spp. (crane flies).

Lepidoptera may likewise be modified using a vector of the invention. Examples of these include, but are not limited to Achoea Janata, Adoxophyes spp., Adoxophyes orana, Agrotis spp. (cutworms), Agrotis ipsilon (black cutworm), Alabama argillacea (cotton leafwonn), Amorbia cuneana, Amyelosis transitella (navel orangeworm), Anacamptodes defectaria, Anomis sabulifera (jute looper), Anticarsia gemmatalis (velvetbean caterpillar), Archips argyrospila (fruittree leafroller), Archips rosana (rose leaf roller), Argyrotaenia spp. (tortricid moths), Argyrotaenia citrine (orange torrid), Autograph gamma, Bongos crunodes, Bourbon cinnabar (rice leaf folder), Bucculatrix thurberiella (cotton leafperforator), Caloptilia spp. (leaf miners), Capua reticulana, Carposina niponensis (peach fruit moth), Chilo spp., Chlurnetia transversa (mango shoot borer), Choristoneura rosaceana (obliquebanded leafroller), Chrysodeixis spp., Cnaphalocerus medinalis (grass leafroller), Colias spp., Conpomorpha cramerella, Cossus cossus (carpenter moth), Crambus spp. (Sod webworms), Cydia funebrana (plum fruit moth), Cydia molesta (oriental fruit moth), Cydia nignicana (pea moth), Darna diducta, Diaphania spp. (stem borers), Diatraea spp. (stalk borers), Diatraea saccharalis (sugarcane borer), Diatraca graniosella (southwester corn borer), Earias spp. (bollworms), Earias insulata (Egyptian bollworm), Earias vitella (rough northern bollworm), Ecdytopopha aurantianum, Elasmopatpus lignosellus (lesser cornstalk borer), Epiphysias postruttana (light brown apple moth), Ephestia spp. (flour moths), Ephestia cautella (almond moth), Ephestia elutella (tobbaco moth), Ephestia kuehniella (Mediterranean flour moth), Epimeces spp., Epinotia aporema, Erionota thrax (banana skipper), Eupoecilia ambiguella (grape berry moth), Euxoa auxiliaris (army cutworm), Feltia spp. (cutworms), Gortyna spp. (stemborers), Grapholita molesta (oriental fruit moth), Hedylepta indicata (bean leaf webber), Helicoverpa spp. (noctuid moths), Helicoverpa armigera (cotton bollworm), Helicoverpa zea (bollworm/corn earworm), Heliothis spp. (noctuid moths), Heliothis virescens (tobacco budworm), Hellula undalis (cabbage webworm), Indarbela spp. (root borers), Keiferia lycopersicella (tomato pinworm), Leucinodes orbonalis (eggplant fruit borer), Leucoptera malifoliella, Lithocollectis spp., Lobesia botrana (grape fruit moth), Loxagrotis spp. (noctuid moths), Loxagrotis albicosta (western bean cutworm), Lyonetia clerkella (apple leaf miner), Mahasena corbetti (oil palm bagworm), Malacosoma spp. (tent caterpillars), Mamestra brassicae (cabbage armyworm), Maruca testulalis (bean pod borer), Metisa plana (bagworm), Mythimna unipuncta (true armyworm), Neoleucinodes elegantalis (small tomato borer), Nymphula depunctalis (rice caseworm), Operophthera brumata (winter moth), Ostrinia nubilalis (European corn borer), Oxydia vesulia, Pandemis ccrasana (common currant tortrix), Pandemis heparana (brown apple tortrix), Papilio demodocus, Peridroma spp. (cutworms), Peridroma sancta (variegated cutworm), Perileucoptera coffeella (white coffee leafminer), Phthorimaea operculella (potato tuber moth), Phyllocnisitis citrella, Phyllonorycter spp. (leaf miners), Pieris rapae (imported cabbageworm), Plathypena scabra, Plodia interpunctella (Indian meal moth), Polychrosis viteana (grape berry moth), Prays endocarpa, Prays oleae (olive moth), Pseudaletia spp. (noctuid moths), Pseudaletia unipunctata (armyworm), Pseudoplusia includens (soybean looper), Rachiplusia nu, Scirpophaga incertulas, Sesamia spp. (stemborers), Sesamia infercns (pink rice stem borer), Sesamia nonagrioides, Setora nitens, Sitotroga cerealella (Angoumois grain moth), Sparganothis ptlleriana, Spodoptera spp. (armyworms), Spodoptera exigua (beet armyworm), Spodoptera fugiperda (fall armyworm), Spodoptera littoralis (cotton leafworm), Spodoptera oridania (southern armyworm), Synanthedon spp. (root borers), Thecla basilides, Thermisia geniniatalis, Tineola bisselliella (webbing clothes moth), Trichoplusia ni (cabbage looper), Tuta absoluta, Yponomeuta spp., Zeuzera coffeae (red branch borer), Zeuzera pyrina (leopard moth), Cydia pomonella (codling moth), Bombyx niori (silk worm), Pectinophora gossypiella (pink bollworm), Plutella xylostella (diamondback moth), Lymantria dispar (Gypsy moth), Amyelois transitella (Navel Orange Worm), Anarsia lineatella (Peach Twig Borer), Tryporyza incertulas (rice stem borer), and Heliothinae spp. (noctuid moths).

Among Coleoptera, examples include, but are not limited to Acanthoscelides spp. (weevils), Acanthoscclides obtectus (common bean weevil), Agrilus planipennis (emerald ash borer), Agriotes spp. (wireworms), Anoplophora glabripennis (Asian longhorned beetle), Anthonomus spp. (weevils), Anthonomus grandis (boll weevil), Aphidius spp., Apion spp. (weevils), Apogonia spp. (grubs), Ataenius spretulus (Black Turgrass Ataenius), Atomaria linearis (pygmy marigold beetle), Aulacophore spp., Bothynoderes punctiventris (beet root weevil), Bruchus spp. (weevils), Bruchus pisorum (pea weevil), Cacoesia spp., Callosobruchus maculatus (southern cow pea weevil), Carpophilus hemipteras (dried fruit beetle), Cassida vittata, Cerosterna spp, Cerotoma spp. (chrysomeids), Cerotoma trifurcata (bean leaf beetle), Ceutorhynchus spp. (weevils), Ceutorhynchus assimilis (cabbage secdpod weevil), Ceutorhynchus napi (cabbage curculio), Chaetocnema spp. (chrysomelids), Colaspis spp. (soil beetles), Conoderus scalaris, Conoderus stigmosus, Conotrachelus nenuphar (plum curculio), Cotinus nitidis (Green June beetle), Crioceris asparagi (asparagus beetle), Cryptolestes ferrugineus (rusty grain beetle), Cryptolestes pusillus (flat grain beetle), Cryptolestes turcicus (Turkish grain beetle), Ctenicera spp. (wireworms), Curculio spp. (weevils), Cyclocephala spp. (grubs), Cylindrocpturus adspersus (sunflower stem weevil), Deporaus niarginatus (mango leaf-cutting weevil), Dermestcs lardarius (larder beetle), Dermestes maculates (hide beetle), Diabrotica spp. (chrysolemids), Epilachna varivestis (Mexican bean beetle), Faustinus cubae, Hylobius pales (pales weevil), Hypera spp. (weevils), Hypera postica (alfalfa weevil), Hyperdoes spp. (Hyperodes weevil), Hypothenemus hampei (coffee berry beetle), Ips spp. (engravers), Lasiodernia serriconie (cigarette beetle), Leptinotarsa decemlineata (Colorado potato beetle), Liogenys futscus, Liogenys suturalis, Lissorhoptrus oryzophilus (rice water weevil), Lyctus spp. (wood beetles/powder post beetles), Maecolaspis joliveti, Megascelis spp., Melanotus communis, Meligethes spp., Meligethes aeneus (blossom beetle), Melolontha mclolontha (common European cockchafer), Oberea brevis, Oberea linearis, Oryctes rhinoceros (date palm beetle), Oryzaephilus mercator (merchant grain beetle), Oryzaephilus surinamensis (sawtoothed grain beetle), Otiorhynchus spp. (weevils), Oulenia nielanopus (cereal leaf beetle), Oulenia oryzae, Pantomorus spp. (weevils), Phyllophaga spp. (May/June beetle), Phyllophaga cuyabana, Phyllotreta spp. (chrysomelids), Phynchites spp., Popillia japonica (Japanese beetle), Prostephanus truncates (larger grain borer), Rhizopertha dominica (lesser grain borer), Rhizotrogus spp. (Eurpoean chafer), Rhynchophorus spp. (weevils), Scolytus spp. (wood beetles), Shenophorus spp. (Billbug), Sitona lineatus (pea leaf weevil), Sitophilus spp. (grain weevils), Sitophilus granaries (granary weevil), Sitophilus oryzae (rice weevil), Stegobiuni paniceuni (drugstore beetle), Tribolium spp. (flour beetles), Tribolium castaneum (red flour beetle), Tribolium confusum (confused flour beetle), Trogoderma variabile (warehouse beetle) and Zabrus tenebioides.

Further, Hemiptera may also be modified with a vector of the invention. Non-limiting examples of Hemiptera that may be so modified, include: Acrosternum hilare (green stink bug), Blissus leucopterus (chinch bug), Calocoris norvegicus (potato mind), Cimex hemipterus (tropical bed bug), Cimex lectularius (bed bug), Dichelops nielacanthus (Dallas), Dagbertus fasciatus, Dichelops furcatus, Dysdercus suturellus (cotton stainer), Edessa meditabunda, Eurygaster maura (cereal bug), Euschistus heron, Euschistus servus (brown stink bug), Helopeltis antonii, Helopeltis theivora (tea blight plantbug), Lagynotomus spp. (stink bugs), Leptocorisa oratorius, Leptocorisa varicomis, Lygus spp. (plant bugs), Lygus hesperus (western tarnished plant bug), Maconellicoccus hirsutus, Neurocolpus longirostris, Nezara viridula (southern green stink bug), Paratrioza cockerelli, Phytocoris spp. (plant bugs), Phytocoris californicus, Phytocoris relativus, Piezodorus Poecilocapsus lineatus (fourlined plant bug), Psallus vaccinicola, Pseudacysta perseae, Scaptocoris castanea, Triatoma spp. (bloodsucking conenose bugs/kissing bugs) and glassy-winged sharpshooters (Honialodisca vitripennis).

Further other insects which may be modified with a vector of the invention. Homoptera, such as Acrythosiphon pisum (pea aphid), Adelges spp. (adelgids), Aleurodes proletella (cabbage whitefly), Aleurodicus disperses, Aleurothrixus floccosus (woolly whitefly), Aluacaspis spp., Amrasca bigutella bigutella, Aphrophora spp. (leafhoppers), Aonidiella aurantii (California red scale), Aphis spp. (aphids), Aphis gossypii (cotton aphid), Aphis fabae (aphid), Aphis pomi (apple aphid), Aulacorthum solani (foxglove aphid), Bemisia spp. (whiteflies), Bemisia argentifolii, Bemisia tabaci (sweetpotato whitefly), Brachycolus noxius (Russian aphid), Brachycorynella asparagi (asparagus aphid), Brevennia rehi, Brevicoryne brassicae (cabbage aphid), Ceroplastes spp. (scales), Ceroplastes rubens (red wax scale), Chionaspis spp. (scales), Chrysomphalus spp. (scales), Coccus spp. (scales), Dysaphis plantaginea (rosy apple aphid), Empoasca spp. (leafhoppers), Eriosoma lanigerum (woolly apple aphid), kerya purchasi (cottony cushion scale), Idioscopus nitidulus (mango leafhopper), Laodelphax striatellus (smaller brown planthopper), Lepidosaphes spp., Macrosiphum spp., Macrosiphum euphorbiae (potato aphid), Macrosiphum granarium (English grain aphid), Macrosiphum rosae (rose aphid), Macrosteles quadrilineatus (aster leafhopper), Mahanarva frimbiolata, Metopolophium dirhoduin (rose grain aphid), Micas longicornis, Myzus persicae (green peach aphid), Nephotettix spp. (leafhoppers), Nephotettix cinctipes (green leafhopper), Nilaparvata lugens (brown planthopper), Parlatoria pergandii (chaff scale), Parlatoria ziziphi (ebony scale), Peregrinus maidis (corn delphacid), Philaenus spp. (spittlebugs), Phylloxera vitifoliae (grape phylloxera), Physokermes piceae (spruce bud scale), Planococcus spp. (mealybugs), Pseudococcus spp. (mealybugs), Pseudococcus brevipes (pine apple mealybug), Quadraspidiotus perniciosus (San Jose scale), Rhapalosiphum spp. (aphids), Rhapalosiphum maida (corn leaf aphid), Rhapalosiphum padi (oat bird-cherry aphid), Saissetia spp. (scales), Saissctia oleae (black scale), Schizaphis graminum (greenbug), Sitobion avenae (English grain aphid), Sogatella furcifera (white-backed planthopper), Therioaphis spp. (aphids), Touineyella spp. (scales), Toxoptera spp. (aphids), Trialeurodes spp. (whiteflies), Trialeurodes vaporariorum (greenhouse whitefly), Trialeurodes abutiloneus (bandedwing whitefly), Unaspis spp. (scales), Unaspis yanonensis (arrowhead scale) and Zulia entreriana.

In some embodiments, the insect is not a Drosophilid such as Drosophila melanogaster. Thus, in some embodiments, expression in Drosophilids, is excluded. In other embodiments, the splice control sequence is not derived from the tra intron of a Drosophilid, especially Drosophila melanogaster.

Species of mosquitoes that may be modified by the constructs of the invention include, but are not limited to mosquitoes of a genera selected from the group consisting of Anopheles sp., Culex sp., Aedes sp., and Toxorhynchites sp. In particular embodiments, the mosquitoes are selected from Anopheles fluviatilis, Culex quinquefasciatus, Anopheles strode, Anopheles pseudopuncti, Aedes aegypti, Anopheles shannoni, Anopheles apicirnaculata, Aedes rubrithorax, Anopheles argyritarsis, Anopheles neoniacultpal, Anopheles flurninensis, Aedes alboannulatus, Aedes albopictus, Anopheles puncarnaculata, Anopheles anornolophyllus, Anopheles vestitipennis, Anopheles albinianus, Toxorhynchites brewpalpie, Toxorhynchites splendens, Toxorhynchites ambionensis, Toxorhynchites rutilus, and Toxorhynchites moctezumai. In specific embodiments, the mosquitoes are selected from the group consisting of Aedes aegypti, Aedes rubrithorax, Aedes albopictus, and Aedes alboannulatus.

### D. Specific Embodiments

In a specific embodiment, a dsx splice control module is used for sex-specific expression in an insect. In this embodiment, the dsx splice control module is derived from Aedes aegypti and incorporates both introns and exons from the Aedes aegypti dsx (Aeadsx). In a preferred embodiment, the Aeadsx splice control module comprises, Exon 4, Intron 5, Exon 5a, Intron 5, Exon 5b, Intron 6 and Exon 6 of the dsx. In a particularly preferred embodiment, portions of the Introns and Exons are used (preserving the splice donor and splice acceptor sites of each) but that are truncated to reduce the size of the overall splice control module.

In some embodiments, the entire sequence of Exon 4 (135 bp) is used, but smaller fragments may be used provided the sequence retains the splice donor site. The Aeadsx Exon 4 is shown as SEQ ID NO:13.

In some embodiments, the entire sequence of Intron 4 is used, but smaller fragments may be used provided the sequence retains the splice donor site. The Aeadsx Intron 4 is large, so it is advantageous to truncate the intron by removing a portion of the middle of the intron such that nucleotides including the splice donor at the 5′ end of the intron are preserved and linked to a 3′ portion of the intron that contains the splice acceptor site. An example of the 5′ end of Intron 4 is shown as SEQ ID NO:12, and an example of the 3′ portion of Intron 4 is shown as SEQ ID NO:11. These are joined to provide a truncated, functional Intron 4.

In some embodiments, the entire sequence of Exon 5a (a female-specific exon) is used, but smaller fragments may be used provided the sequence retains the splice donor site. The Aeadsx Exon 5a is shown as SEQ ID NO:6.

In some embodiments, the entire sequence of Intron 5 (209 bp) is used, but smaller fragments may be used provided the sequence retains the splice donor site. The Aeadsx Intron 5 is shown as SEQ ID NO:10.

In some embodiments, a protein encoding portion of Exon 5b (a female-specific exon) is used. In the native exon, only 56 nucleotides are protein encoding. In some embodiments, the Exon 5b is engineered to open the reading frame so that the entire sequence is protein encoding. This may be done by any manipulation that puts the sequence in frame and retains as much native primary amino acid sequence as will be functional. In one such manipulation (relative to the native Aedes aegypti dsx gene (shown as SEQ ID NO:7) a total of 5 nucleotide insertions are made, a single nucleotide is deleted and a single nucleotide change was made to obtain a protein encoding Exon 5b.

In some embodiments, the entire sequence of Intron 6 is used, but smaller fragments may be used provided the sequence retains the splice donor site. The Aeadsx Intron 6 is large, so it is advantageous to truncate the intron by removing a portion of the middle of the intron such that nucleotides including the splice donor at the 5′ end of the intron are preserved and linked to a 3′ portion of the intron that contains the splice acceptor site. An example of the 5′ end of Intron 6 is shown as SEQ ID NO:9, and an example of the 3′ portion of Intron 6 is shown as SEQ ID NO:8. These are joined to provide a truncated, functional Intron 6.

In some embodiments, the entire sequence of Exon 6 (a shared exon) is used, but smaller fragments may be used provided the sequence retains the splice acceptor site. In other embodiments, only a 5′ portion of the Exon 6 is used. An example of a 5′ portion of Exon 6 that may be used is shown as SEQ ID NO:5.

In a specific example, the Splice Control Module is an Aeadsx comprising Exon 4 (SEQ ID NO:13), a truncated Intron 4 (composed of SEQ ID NO:12 and SEQ ID NO:13), Exon 5a (SEQ ID NO:6), Intron 5 (SEQ ID NO:10), a modified Exon 5b (SEQ ID NO:7), a truncated Intron 6 (composed of SEQ ID NO:9 and SEQ ID NO:8) and Exon 6 (SEQ ID NO:5).

As with all nucleotide sequences discussed herein, it is preferred that a certain degree of sequence homology is envisaged, unless otherwise apparent. Thus, it is preferred that the elements of the Aeadsx splice control module has at least 80%, 85%, 90%, 95%, 99% or 99.9% sequence homology with the reference sequences SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13. A suitable algorithm such as BLAST may be used to ascertain sequence homology. If large amounts of sequence are deleted compared to the wild type, then the sequence comparison may be over the full length of the wild type or over aligned sequences of similar homology.

In a specific embodiment of the invention, the control factor is the tTA gene product or an analogue thereof, and wherein one or more tetO operator units is operably linked with the promoter and is the enhancer, tTA or its analogue serving to enhance activity of the promoter via tetO. It is preferred that functional protein encodes the tTAV or tTAV product and preferably, the promoter is substantially inactive in the absence of the positive transcriptional control factor. Suitable, preferably minimal, promoters for this system can be selected from: hsp70, a P minimal promoter, a CMV minimal promoter, an Act5C-based minimal promoter, a BmA3 promoter fragment, a promoter fragment from hunchback, an Adh core promoter, and an Act5C minimal promoter, or combinations thereof. In some embodiments, the functional protein itself a transcriptional transactivator, such as the tTAV system.

### E. Methods of Biological Control

In a further aspect, there is also provided a method of population control of an organism in a natural environment therefor, comprising:
i) breeding a stock of the organism, the organism carrying a gene expression system comprising a system according to the present invention which is a dominant lethal genetic system,
ii) distributing the said stock animals into the environment at a locus for population control; and
iii) achieving population control through early stage lethality by expression of the lethal system in offspring that result from interbreeding of the said stock individuals with individuals of the opposite sex of the wild population.

Preferably, the early stage lethality is embryonic or before sexual maturity, preferably early in development, most preferably in the early larval or embryonic life stages.

Preferably, the lethal effect of the lethal system is conditional and occurs in the said natural environment via the expression of a lethal gene, the expression of said lethal gene being under the control of a repressible transactivator protein, the said breeding being under permissive conditions in the presence of a substance, the substance being absent from the said natural environment and able to repress said transactivator.

Preferably, the lethal effect is expressed in the embryos of said offspring. Preferably, the organism is an invertebrate multicellular animal or is as discussed elsewhere.

Also provided is a method of biological control, comprising:
i) breeding a stock of males and female organisms transformed with the expression system according to the present invention under permissive conditions, allowing the survival of males and females, to give a dual sex biological control agent;
ii) optionally before the next step imposing or permitting restrictive conditions to cause death of individuals of one sex and thereby providing a single sex biological control agent comprising individuals of the other sex carrying the conditional lethal genetic system;
iii) releasing the dual sex or single sex biological control agent into the environment at a locus for biological control; and
iv) achieving biological control through expression of the genetic system in offspring resulting from interbreeding of the individuals of the biological control agent with individuals of the opposite sex of the wild population.

Preferably, there is sex-separation prior to organism distribution by expression of a sex specific lethal genetic system.

Preferably, the lethal effect results in killing of greater than 90% of the target class of the progeny of matings between released organisms and the wild population.

Also provided is a method of sex separation comprising:
i) breeding a stock of male and female organisms transformed with the gene expression system under permissive or restrictive conditions, allowing the survival of males and females; and
ii) removing the permissive or restrictive conditions to induce the lethal effect of the lethal gene in one sex and not the other by sex-specific alternative splicing of the lethal gene.

Preferably, the lethal effect results in killing of greater than 90% of the target class of the progeny of matings between released organisms and the wild population.

Also provided is a method to selectively eliminate females from a population. The equivalent for males is also envisaged.

The invention will now be described by reference to the following examples which are meant to be illustrative of embodiments of the invention and are not to be construed as limiting the invention.

### EXAMPLES

### Example 1

Genetically engineered Aedes aegypti strains were generated by insertion of the recombinant DNA (rDNA) construct (FIG. 1) (hereinafter “DSX-tTAV-Red”) into the Ae. aegypti genome. This DNA is comprised of two gene cassettes contained between the 5′ and 3′ fragments of the Trichoplusia ni piggyBac transformation system used to insert them into the insect genome. The gene cassettes are as follows:
1. The Hr5IE1 enhancer and promotor, which derives from Autographa californica nuclear polyhedrosis virus (AcNPV), drives expression of the DsRed2 protein. This protein is a synthetic derivative of a red fluorescent protein sourced from Clontech.
2. A minimal promoter from the Drosophila melanogaster heat shock protein 70 ( [symbol] minipro) gene, downstream of a tetracycline responsive operator (TetO x7), drives expression of the synthetic tetracycline-repressive transcriptional activator protein (tTAV) (Gossen and Bujard 1992) Proc. Natl. Acad. Sci. USA 89(12):5547-5551; Gong et al., (2005) Nat. Biotech. 23:453-456). Expression of tTAV protein is rendered female-specific by the inclusion of portions of the Ae. aegypti doublesex gene (Aeadsx). As the Aeadsx sequences will lead to additional amino acids included on the N-terminus of the tTAV protein, the ubiquitin protein (Ubi) is placed between the Aeadsx and tTAV sequences. Ubiquitin is cleaved through normal cellular processes, and so the Aeadsx-derived and Ubi amino acids are removed, leaving tTAV (Bachmair et al., (1986) Science 234(4773):179-186; Varshaysky, A. (2005) Meth. Enzyinol. 399:777-799).

Together these gene cassettes deliver a strain of Ae. aegypti that, when reared in the presence of tetracycline, development occurs normally in both sexes, but when reared in the absence of tetracycline females do not survive to adulthood and a male-only cohort is produced. Additionally, each insect is marked with the fluorescent DsRed2 protein.

A. Preparation of the Aeadsx Splice control module

The Aeadsx splice control module was engineered from endogenous components of the Ae. aegypti doublesex gene that normally give rise to the sex-specific alternative splicing of the gene. These are exons 4, 5a, 5b and 6; and introns 4, 5 and 6. The sequences of some of these components (introns 4, intron 6 and exon 5b) were manipulated before being integrated in the sex-specific module.

Introns 4 and 6 are too large to be included full length in pDSX-TTAV-RED, natively 14.526 kb and 10.393 kb, respectively. The 5′ and 3′ ends of each intron have been retained, but the central intronic sections removed without losing functionality. The final sizes in the Aeadsx splice control module were 1.750 kb for intron 4, and 1.446 kb for intron 6.

Exon 5b, that partially encodes in the native protein, was modified to allow an open reading frame to span the entire exon so that it would be in-frame with the Leading Peptide (e.g., ubiquitin) encoding sequence which was likewise in frame with a gene of interest. This was accomplished by making one base pair substitution, deleting 1 base pair and inserting 5 base pairs. This manipulation made the reading frame in the F2 transcript protein coding from the engineered start codon (ATG) immediately upstream of exon 4, through exons 4, 5b and 6, and into Leading Peptide/gene of interest. The M (male) splice form includes exon 6 which when spliced to exon 4 causes a frame shift which results in the inclusion of a stop codon prior to the gene of interest coding sequence.

a. Aeadsx Control of tTAV

To generate DSX-tTAV-Red, the Aeadsx splice control module described above was engineered to produce a synthetic, repressible transcriptional activator protein, tTAV. It was engineered to be under the control of a tetracycline responsive composite promoter, engineered by joining 7 repeats of TetO operator sequence from E. coli (TetO x7) with a minimal promoter from the heat shock protein 70 gene of Drosophila melanogaster (DmHsp70 minipro) (Gossen & Bujard, 1992; Gong et al., 2005). The tTAV then acts in a positive feedback loop as the binding of tTAV to TetO drives further expression of that same protein. Without wishing to be bound by any particular theory of operation, it is believed that high level expression is deleterious to cells, likely due to transcriptional “squelching” (Gill and Ptashe, 1988). This feedback loop can be broken by the administration of tetracycline as this molecule is bound by tTAV and thereby rendered unable to bind the operator, TetO.

The feedback loop operates specifically in females due to the addition Aeadsx) splice control module wherein the mRNAs produced in males and females are different due to sex-specific splicing. This, in turn means that the tTAV protein is only correctly encoded by an mRNA produced in females. Only the F2 splice form correctly encodes the tTAV protein.

In addition to the Aeadsx module, the self-limiting gene encodes the D. melanogaster Ubiquitin (Ubi) protein as an N-terminal fusion of the tTAV protein. In the insect, Ubi is predicted to be precisely processed post translation of the protein in order to remove any parts of the polypeptide encoded by the Aeadsx splice control module to leave just the tTAV protein without any further polypeptide sequences at the N-terminus of the protein (Bachmair et al., (1986) Science 234(4773):179-186; Varshaysky, A. (2005) Meth. Enzymol. 399:777-799). Ubiquitin is encoded as a polypeptide in eukaryotes including insects, and relies on precise proteolytic cleavage at the C-terminal residue of the ubiquitin 76-mer sequence to generate free ubiquitin. By fusing alternative sequences to the C-terminus of Ubiquitin, it is possible to take advantage of this cleavage activity to cleave a protein of interest from an N-terminal tag.

### B. Preparation of the DsRed2 Cassette

DSX-TTAV-RED contains a Hr51E1 enhancer and promotor that drives expression of a DsRed2 protein. The fluorescent phenotype is clearly visible in all larval, pupal and adult life stages. This provides an enhancement of the phenotype of self-limiting strain, OX513A, that utilises the Actin5C promoter to produce DsRed2 markings in larval and pupal stages.

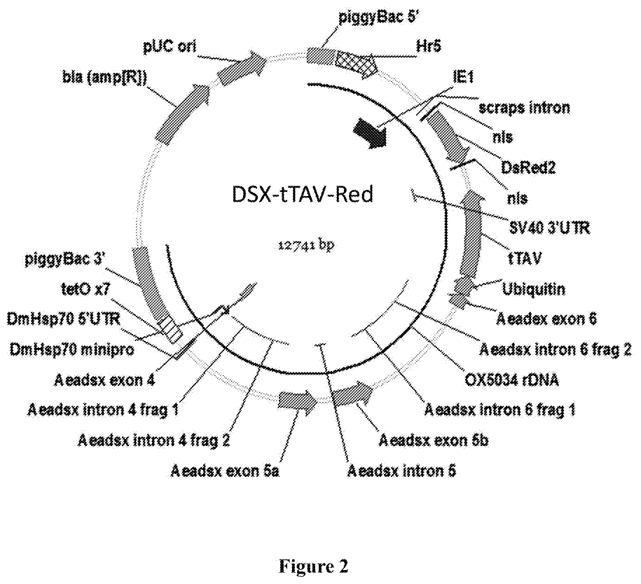

Nuclear localization signal peptide encoding sequences (nls1 and nls2) were engineered on the N-terminal and C-terminal encoding portions of DsRed2 as shown in FIG. 2 based on SV40 NLS sequence (Kalderon et al. (1984) Cell 39(3):499-509). In this example, the nls sequences are shown in SEQ ID NO:20 (nls1) and SEQ ID NO:21 (nls2).

### C. Preparation of the Vector Plasmid

The vector plasmid shown in FIG. 2 is based on cloning vector pKC26-FB2 (Genbank #HQ998855). The plasmid backbone contains the pUC origin of replication and the beta-lactamase ampicillin resistance gene for use in molecular cloning procedures. This plasmid section is not included in the rDNA or incorporated into the insect genome. The vector plasmid also contains the complete rDNA that is incorporated into the insect and includes; the 3′ and 5′ piggyBac element ends derived from Trichoplusia ni, the DsRed2 Cassette containing the gene for the DsRed2 red fluorescence marker protein from Diciyosonia, a synthetic DNA sequence for the tetracycline repressible transcriptional activator tTAV based on a fusion of sequences Gossan and Bujard (1992) Proc. Natl. Acad. Sci. USA 89(12):5547-5551, and a modified Aeadsx splice control module derived from Ae. aegypti (as described above and generally throughout the Specification). A table of the components shown in FIG. 2 is shown in Table 1 where the nature of each component is also described. The plasmid was prepared using routine DNA cloning procedures.

TABLE 1

| | | Location (bp) in | Size | | SEQ |
|---|---|---|---|---|---|
| Component | Source | DSX-tTAV-RED | (bp) | Function | ID NO: |
| piggyBac 5′ | Synthetic (Derived | 1-309 | 309 | Facilitates germline transformation with rDNA | 16 |
| | from Trichoplusia ni) | | | only in the presence of the piggyBac | |
| | | | | transposase. (Carry et al. (1989) Virol. | |
| | | | | 172(1): 156-169, Fraser et al. (1995) Virol. | |
| | | | | 211: 397-407, Fraser et al. (1996) Insect Mol. Biol. | |
| | | | | 5: 141-151). | |
| Hr5 | Baculovirus | 355-863 | 509 | Transcriptional enhancer to stimulate expression | 17 |
| | nucleopolyhedrovirus | | | from the IE1 promoter (Rodems and Friesen | |
| | (AcNPV) | | | (1993) J. Virol. 69(10): 5776-5785) | |
| IE1 | Baculovirus | 924-1553 | 630 | Promoter to drive the expression of DsRed2 | 18 |
| | Autographa | | | protein (Guarino and Dong (1991) J. Virol. | |
| | californica | | | 65(7): 3676-3680) | |
| | nucleopolyhedrovirus | | | | |
| | (AcNPV) | | | | |
| Scraps intron | Drosophila | 1569-1631 | 63 | An intron cloned upstream of the DsRed2 coding | 19 |
| | melanogaster | | | sequence to facilitate transcription of mRNA | |
| | | | | (Field et al. (2005) Development 132(12): | |
| | | | | 2849-2860 | |
| nls | Synthetic sequence | 1668-1709 | 42 | nls: Nuclear Localisation Signal. Synthetic DNA | 20 |
| | | and | and | sequences that encode protein domains at the | |
| | | 2397-2429 | 33 | N- and C-terminal ends of DsRed2 for import into | |
| | | | | the cell nucleus by importins (Lange (2007) J. | |
| | | | | Biol. Chem. 282: 5101-5105 | |
| DsRed2 | Synthetic DNA | 1716-2390 | 675 | Marker gene-a red fluorescent protein | 4 |
| | encoding a variant | | | (Lukyanov et al. (2000) J. Biol. Chem. | |
| | of red fluorescent | | | 2755: 25879-25882; Matz et al. (1999)Nat. | |
| | protein (Clontech) | | | Biotechnol. 17(10): 969-973) | |
| SV40 3′UTR | Synthetic non- | 2455-2682 | 228 | A 3′ untranslated sequence. It contains the | 22 |
| | coding fragment | | | transcription termination and polyadenylation | |
| | based on Simian | | | signals (Clontech Laboratories Inc. 2012 available | |
| | virus (SV40) | | | on the Clontech Website; Brand and Perrimon | |
| | isolated from | | | (1993) Development 118: 401-415) | |
| | pDsRed2-N1 | | | | |
| | (Clontech plasmid) | | | | |
| tTAV | Synthetic fusion | 2715-3725 | 1011 | Tetracycline repressible transcription factor | 3 |
| | tetracycline | | | (Gossen and Bujard (1992) Proc. Natl. Acad. Sci. | |
| | transactivator | | | USA 89(12): 5547-5551; Gong et al. (2005) Nat. | |
| | protein. Optimised | | | Biotech. 23: 453-456). | |
| | for expression in | | | | |
| | insects. | | | | |
| Ubiquitin | Drosophila | 3726-3950 | 225 | Stimulates cleavage of tTAV protein from the | 2 |
| | melanogaster | | | Aeadsx-Ubi that is N-terminally fused | |
| | | | | (Varshavsky, (2005)Meth. Enzymol. 399777-799) | |
| Aeadsx splice | Aedes aegypti | 3951-4101 | 151 | Aeadsx exon 6: 5′end of the native Aeadsx exon 6. | 5 |
| control module | | | | This is a shared exon. | |
| | | 4100-4987 | 886 | Aeadsx intron 6 frag2: The 3′end of intron 6. | 8 |
| | | | | This fragment is used with Aeadsx intron 6 frag1 | |
| | | | | to build the truncated version of intron 6. | |
| | | 4990-5547 | 560 | Aeadsx intron 6 frag1: The 5′end of intron 6. | 9 |
| | | | | This fragment is used with Aeadsx intron 6 frag2 | |
| | | | | to build the truncated version of intron 6. | |
| | | 5548-6016 | 469 | Aeadsx exon 5b: An engineered version of the | 7 |
| | | | | native exon 5b. To open the reading frame | |
| | | | | throughout the whole exon (only 110 nt are | |
| | | | | coding in the native exon), a total of 4 insertions, | |
| | | | | 1 nt deletion and 1 nt change were introduced to | |
| | | | | this exon. This is a female-specific exon. | |
| | | 6017-6225 | 209 | Aeadsx intron 5: The whole sequence of the | 10 |
| | | | | native intron 5. | |
| | | 6226-6683 | 458 | Aeadsx exon 5a: The whole sequence of the | 6 |
| | | | | native exon 5a. This is a female-specific exon. | |
| | | 6684-7267 | 584 | Aeadsx intron 4 frag2: The 3′end of intron 4. | 11 |
| | | | | This fragment is used with Aeadsx intron 4 frag1 | |
| | | | | to build the truncated version of intron 4. | |
| | | 7268-7858 | 591 | Aeadsx intron 4 frag1: The 5′end of intron 4. | 12 |
| | | | | This fragment is used with Aeadsx intron 4 frag2 | |
| | | | | to build the truncated version of intron 4. | |
| | | 7859-7993 | 135 | Aeadsx exon 4: The whole sequence of the | 13 |
| | | | | native Aeadsx exon 4. This is a shared exon. | |
| DmHsp70 minipro | Drosophila | 8002-8131 | 130 | The minimal promoter (43 bp) and the 5′UTR (87 | 23 |
| | melanogaster | | | bp) from the hsp70 gene promotes expression | |
| | | | | when the tTAV is bound to the neighbouring | |
| | | | | TetO operator. | |
| TetO x7 | Synthetic DNA | 8137-8432 | 296 | Binds tTAV in the absence of tetracycline, | 14 |
| | contains 7 repeats | | | facilitating expression by the neighbouring mini- | |
| | of Tn10 tet-operon | | | promoter (Gossen and Bujard 1992). | |
| piggyBac 3′ | Synthetic (Derived | 8461-9325 | 865 | Facilitates germline transformation with rDNA | 15 |
| | from Trichoplusia ni) | | | only in the presence of the piggyBac transposase. | |

### D. Strain Generation

For insertion of the cassettes into Aedes aegypti, Aedes aegypti of the Latin wild type strain (originating from Mexico) were reared under standard insectary conditions [26° C.±2° C.], 70% [±10%] relative humidity and 12 h:12 h light:dark cycle. Mosquito embryos were transformed by standard micro-injection methods (Jasinskiene et al., (1998) Proc. Natl. ACad. Sci. USA 95:7520-7525; Morris, A. C. (1997) “Microinjection of mosquito embryos” In: Crampton, J. M., Beard, C. B., Louis, C. (Eds.), MOLECULAR BIOLOGY OF INSECT DISEASE VECTORS: A METHODS MANUAL. Chapman & Hall, 2-6 Boundary Row, London SE1 8HN, UK, pp. 423-429), injecting a combination of plasmid DNA (concentration of 300 ng/μl of DSX-tTAV-Red, the plasmid depicted in FIG. 2), and piggyBac mRNA (at a concentration of 500 ng/μl) as the source of transposase. The plasmid DNA and the transposase mRNA were reconstituted in an injection buffer (5 mM KCl, 0.1 mM NaH₂PO₄, pH 6.8) made using standard laboratory grade reagents (Handler and James, 1998).

### E. Strain Selection

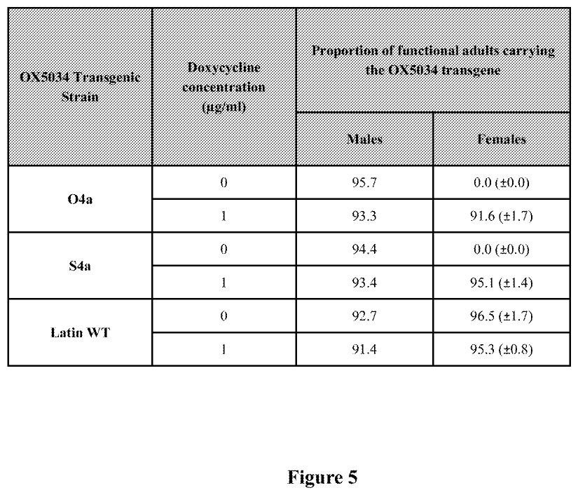

Selection of a transgenic DSX-TTAV-RED strain was carried out by DSX-tTAV-Red adult injection survivors (Generation 0 or G₀) were back crossed to Latin WT. Two G₀ males were crossed to 10 Latin WT females and 6 G₀ females were crossed with 6 Latin WT males. G₁ pupae were screened for DsRed2 fluorescence using a Leica M80 microscope equipped with filters for detection: maximum excitation 563 nm, emission 582 nm. Ten G₁ transgenic families were obtained (8 from male G₀ crosses, 2 from female G₀ crosses) from which 3 individual G₂ males were crossed to WT females, resulting in 20 transgenic strains producing viable eggs. Strains were maintained by crossing G₃ males to Latin WT females. G₄ hemizygous progeny from all strains were assessed for their survivability when reared in the presence and absence of antidote (doxycycline hyclate). Fifteen strains presented the desired phenotype; an unbiased sex ratio in the presence of doxycycline and complete female penetrance in the absence of doxycycline (i.e., no female survival). Strains not showing this phenotype were discarded. Assessment of adult eclosion and survival of individuals carrying the DSX-tTAV-Red transgene suggests that pupae from these 15 strains can successfully eclose into adults. Results from the two relevant strains (O and S) are displayed in FIG. 5.

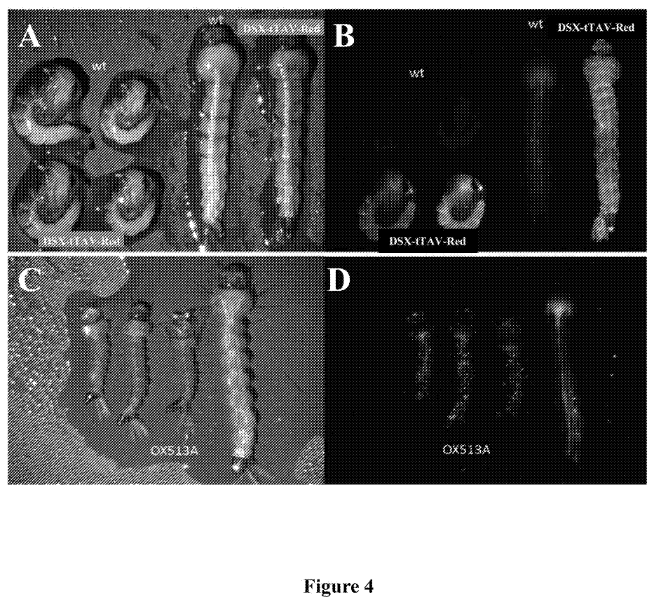

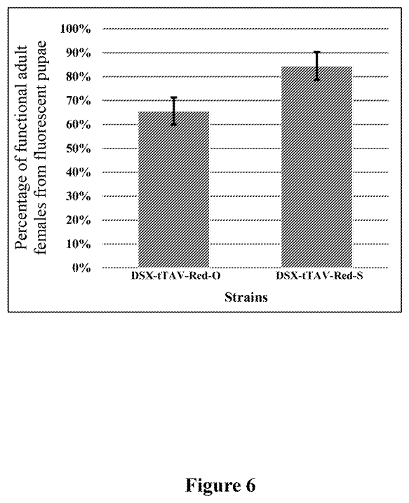

Assessment of the potential zygosity of the strains was carried out by screening the pupae from G₅ hemizygous crosses according to Mendelian genetics (3:1 ratio fluorescent: non fluorescent) where homozygotes are expected to have a brighter fluorescent phenotype. Nine strains presenting with over 10% survival of potential homozygotes were selected for eclosion assessment. Results from the two strains (DSX-tTAV-Red-O and DSX-tTAV-Red-S) are shown in FIG. 6. Nine strains were found to have the expected Mendelian inheritance ratios and these were further evaluated by crossing male and females expressing the bright phenotype (families of 1 male to 2 females) and PCR analysis (genotyping). Progeny of the crosses were screened for fluorescence over 2 generations (Representative Fluorescent larval/pupal stages shown in FIG. 4) resulting in a homozygous DSX-tTAV-Red-O substrain from 16 parents (7 male and 9 female) and DSX-tTAV-Red-S strain from 70 parents (29 male and 41 female). Despite the fact that integration occurred in the same position within the mosquito chromosome for each transgenic event, only substrains O and S showed the desired phenotype. The mosquitoes are not inbred, so there are small variations in the chromosome near the site of integration which may influence whether the inserted genetic material functions or does not. Therefore, we assessed the precise integration point of the vector and designed an assay to detect divergence in the mosquitoes and approximate the variations present in the Aedes aegypti population.

To ensure DSX-tTAV-Red homozygotes successfully eclosed into viable adults, survivorship was assessed. Of the fluorescent pupae, the expected ratio of hemizygotes to homozygotes would be 2:1, according to Mendelian genetics. Two strains, O and S were identified as suitable candidates, with the proportion of potential homozygotes being 15.6% and 34.4%, respectively (FIG. 6). Strain S4a was later found to contain two insertions of the DSX-TTAV-RED transgene. These were separated prior to homozygosis of the strain.

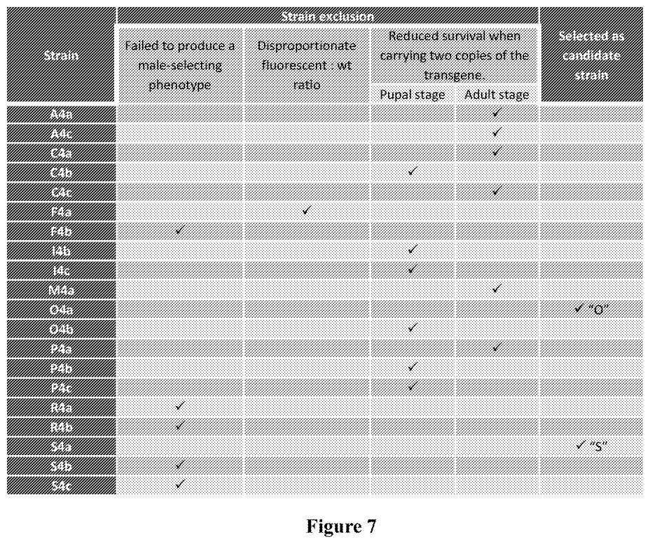

Not all strains tested satisfied all criteria. While not wishing to be bound by any particular the transgenic event and insertion into particular areas of the Aedes aegypti chromosome influences the expression of the gene and observed phenotype. The summary of the strain selection is shown in FIG. 7.

### Example 4

### Protocol for Detection of DSX-tTAV-Red Transgene

This assay was used to detect the presence or absence of the DSX-tTAV-Red transgene in a variety of DSX-tTAV-Red insect samples (field, mass-rearing and laboratory). The same protocol can also be used to provide evidence of stability of the DSX-tTAV-Red transgene over time. Successful amplification of the DSX-tTAV-Red transgene over time provides evidence of its stability, as one primer anneals to the transgene, the other to the flanking genomic sequence, so mobilisation of the transgene results in a negative PCR.

a. Extraction of Genomic DNA

Genomic DNA was isolated from individual insects using the protocol below using the Invitrogen Purelink™ genomic extraction kit.

A solution of 96-100% ethanol is added to PureLink™ Genomic Wash Buffer and PureLink™ Genomic Wash Buffer 2 according to Instructions on each label (Invitrogen) and mixed well.

180 μL of PureLink™ Genomic Digestion Buffer and 20 μL Proteinase K is added to each pool of abdomens. The insect samples are broken up with a sterile pestle, ensuring that the tissue is completely immersed in the buffer mix. The solutions are incubated at 55° C. with occasional vortexing until lysis is complete (1-4 hours). Alternatively, the samples may be placed overnight to digest.

The samples are centrifuged at maximum speed for 3 minutes at room temperature to remove any particulate materials, and the supernatant is transferred to a new microcentrifuge tube. 20 μL RNase A is added to lysate, and mixed well by briefly vortexing, then incubate at room temperature for 2 minutes.

200 μL PureLink™ Genomic Lysis/Binding Buffer is added and mixed well by vortexing to yield a homogenous solution. 200 μL 96-100% ethanol is then added to the lysate. The lysates are mixed well by vortexing to yield a homogenous solution. Alternatively, the Lysis/binding buffer and 100% Ethanol can be mixed before adding.

The lysate (−640 μL) prepared with PureLink™ Genomic Lysis/Binding Buffer and ethanol is added to the PureLink™ Spin Column in a Collection Tube from the kit.

The columns are then centrifuged at 10,000×g for 1 minute at room temperature. The collection tube is discarded and the spin column is placed into a clean PureLink™ Collection Tube supplied with the kit.

500 μL Wash Buffer 1 prepared with ethanol is added to the column and the column is centrifuged at 10,000×g for 1 minute at room temperature. The collection tube is discarded and the spin column is placed into a clean PureLink™ Collection Tube supplied with the kit.

500 μL of Wash Buffer 2 prepared with ethanol is added to the column and the column is centrifuged at maximum speed for 3 minutes at room temperature. The flow through is discarded and the column is re-spun for a further minute at 10,000×g.

The spin column is placed in a sterile 1.5-ml microcentrifuge tube and 100 μL of PureLink™ Genomic Elution Buffer is added to the column. The column is incubated at room temperature for 1 minute then centrifuged at maximum speed for 1 minute at room temperature. The column is then removed and discarded and the purified DNA collected is used or stored the purified DNA at 4° C. (short-term) or −20° C. (long-term).

It was found that for the DSX-TTAV-RED-O strain, the DSX-TTAV-RED rDNA was inserted at supercont1.420 position 324552, between gene AAEL009696 and an exon in gene AAEL009706, while for the DSX-TTAV-RED-S strain, the DSX-TTAV-RED rDNA was inserted at supercont1.19 position 2799615, also known as contig AAGE02001348.1 position 88740.

For the DSX-TTAV-RED-O strain, the insertion of DSX-TTAV-RED rDNA in the Aedes aegypti genome, the rDNA is inserted in a region corresponding to a sequence of SEQ ID NO: 46 between nucleotides 1845 and 1850. The rDNA insert is flanked on the 5′ end by sequence of the Aedes aegypti genome corresponding to nucleotides 1443 to 1845 of SEQ ID NO: 46 (SEQ ID NO: 73) and on the 3′ end by sequence of the Aedes aegypti genome corresponding to nucleotides 1859 to 2222 of SEQ ID NO: 46 (SEQ ID NO: 74).

For the DSX-TTAV-RED-S strain, the insertion of DSX-TTAV-RED rDNA in the Aedes aegypti genome, the rDNA is flanked on the 5′ end by sequence of the Aedes aegypti genome corresponding to SEQ ID NO: 75 and on the 3′ end by sequence of the Aedes aegypti genome corresponding to SEQ ID NO: 76.

### PCR for Genotyping DSX-tTAV-Red-O and DSX-tTAV-Red-S

### A) DSX-tTAV-Red-O

Genotyping for DSX-tTAV-Red-O strain was carried out by Taqman real-time PCR. The DSX-tTAV-Red transgene is quantified by normalising qPCR Ct values to an internal reference gene, IAP. Relative copy number of the DSX-tTAV-Red transgene is calculated by comparing the normalised Ct values for DSX-tTAV-Red in the (known heterozygote) calibrator sample with that in each unknown sample. The relative copy number of DSX-tTAV-Red rDNA in the unknown samples are expected to be ˜1 for hemizygotes and ˜2 for homozygotes, although inefficiencies in amplification under these conditions led to individuals with relative DSX-tTAV-Red copy number >1.2 being considered to be homozygotes.

The PCR was carried out using TaqMan● Gene Expression Master Mix (ThermoFisher Scientific) under the following conditions: initial denaturation and enzyme activation at 95° C. for 10 mins, 43 cycles of denaturation at 94° C. for 11 s, probe annealing at 60° C. for 15 s, primers annealing at 54° C. for 30 s and extension at 60° C. for 30 s.

| Oligonucleotide | | Sequence | Fluorescent |
|---|---|---|---|
| name | Target | (SEQ ID NO) | quencher/label |
| 711-VP16taqF | DSX-tTAV- | CATGCCGACGCGCTAGA | N/A |
| | Red-O | (SEQ ID NO: 47) | |
| 712-VP16taqR | DSX-tTAV- | GGTAAACATCTGCTCAAACTCGAAGTC | N/A |
| | Red-O | (SEQ ID NO: 48) | |
| 2131-VP16probe2 | DSX-tTAV- | FAM-TCGATCTGGACATGTTGGGGGACG- | BHQ1, FAM |
| | Red-O | BHQ1 | |
| | | (SEQ ID NO: 49) | |
| SS1752-AedesF | IAP | CTGCAGTAGTGATGAAGATGAACCA | N/A |
| | | (SEQ ID NO: 50) | |
| SS1753-AedesR | IAP | GGGCGAAAATGCCGTATTGTACTCA | N/A |
| | | (SEQ ID NO: 51) | |
| SS1884-AedesPro | IAP | HEX- | BHQ1, HEX |
| | | AGACACCAGTCGGACTTGCAAAATCTG- | |
| | | BHQ1 | |
| | | (SEQ ID NO: 52) | |

### B. DSX-tTAV-Red-S

Genotyping for DSX-tTAV-Red-S was carried out by endpoint PCR using the oligonucleotides shown below and the following PCR conditions: 94° C., 2 min; 3-5 cycles of 94° C. 15 s, 60° C. 30 s −0.5° C./cycle, 72° C. 15 s; 23 cycles of 94° C. 15 s, 55° C. 30 s, 72° C. 15 s; 72° C. 7 min; 4° C. hold. Wild-type PCR product (240 bp) was the result of amplification with primers SS2326)5034S5R1 and SS2336)5034S3F2. DSX-tTAV-Red PCR product (221 bp) was the result of amplification with primers SS2326)5034S5R3 and TD225)Mod-666-sal.

| Oligonucleotide | | | |
|---|---|---|---|
| name | Target | Sequence | SEQ ID NO |
| SS2326 | DSX-tTAV- | GCTTCATTAAGCAGAAACACTGA | SEQ ID NO: 53 |
| | Red-S | | |
| | 5′ flanking | | |
| | sequence | | |
| TD225)Mod-666- | DSX-tTAV- | TGACAAGCACGCCTCACGGGAG | SEQ ID NO: 55 |
| sal | Red-S | | |
| | transgene | | |
| SS2336)5034S3F2 | DSX-tTAV- | CATCTAACTCTACTTTGTGTGGGAATCA | SEQ ID NO: 54 |
| | Red-S | | |
| | 3′ flanking | | |
| | sequence | | |

Transgene specific gene sequences are amplified by PCR using PCR BIO polymerase as follows:

To amplify the insert in the DSX-tTAV-RED-O substrain, two specific oligonucleotide primers were designed: TD4037 (5′-CTGTTGCTGCGCACGAAACAC-3′; SEQ ID NO: 38) which anneals to the Aedes aegypti genomic DNA, and TD2127 (5′-GTGCCAAAGTTGTTTCTGACTGAC-3′; SEQ ID NO: 39) which anneals to the inserted construct in a region shown in SEQ ID NO: 36. This primer set only amplifies samples containing the DSX-tTAV-RED-O transgene (data not shown).

To amplify the insert in the DSX-tTAV-RED-S substrain, two specific oligonucleotide primers were designed: TD4037 (5′-TGACAAGCACGCCTCACGGGAG-3′; SEQ ID NO:41) which anneals to the Aedes aegypti genomic DNA, and TD225 (5′-GCTTCATTAAGCAGAAACACTGA-3′; SEQ ID NO: 40) which anneals to the inserted construct in a region shown in SEQ ID NO: 37, and produces a product of 221 bp. This primer set only amplifies samples containing the DSX-tTAV-RED-S transgene.

Other methods for amplifying and detecting the transgene are as follows:

### 1. Endpoint PCR Detection Methods

1.1 Detection of DSX-tTAV-Red rDNA

In the first method, PCR primers may be designed such that one primer anneals within the DSX-tTAV-Red rDNA and the other primer anneals to Aedes aegypti DNA in the region flanking the insertion site of the OX5034 rDNA. Such primers can be as close together or as far away (in terms of bp) from one another as desired. For convenience, we generally design for amplicons that are 200-500 bp for agarose gel analysis, 200 bp for qPCR with SybrGreen, and 100 bp for Taqman qPCR.

These primers may be used to amplify a suitable PCR amplicon which may be detected by agarose gel DNA electrophoresis using an intercalating dye such as ethidium bromide.

For example, DSX-tTAV-Red-O rDNA may be detected using the following primers:

| | Primer ID | Sequence |
|---|---|---|
| Primer 1 | 4039)5034Ofla4 | CTGTTGCTGCGCACGAAACAC |
| | | (SEQ ID NO: 56) |
| Primer 2 | 2127)PB4-2 | GTGCCAAAGTTGTTTCTGACTGAC |
| | | (SEQ ID NO: 57) |

For example, DSX-tTAV-Red-S rDNA may be detected using the following primers:

| | Primer ID | Sequence |
|---|---|---|
| Primer 1 | SS2326)5034S5R3 | GCTTCATTAAGCAGAAACACTGA |
| | | (SEQ ID NO: 53) |
| Primer 2 | TD225)Mod-666- | TGACAAGCACGCCTCACGGGAG |
| | sal | (SEQ ID NO: 55) |

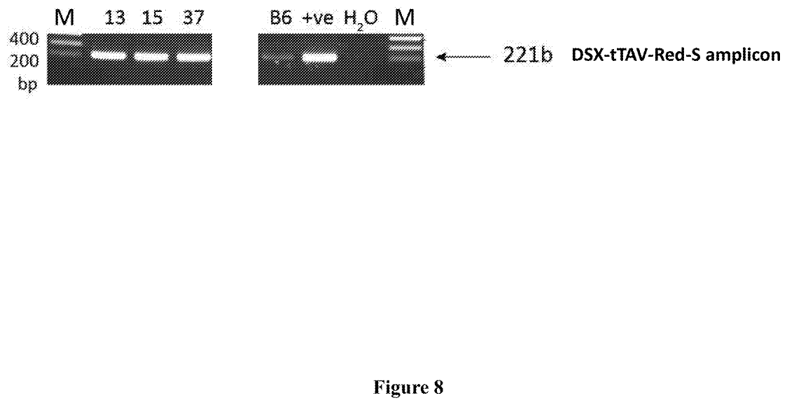

A representative gel showing amplification of PCR products using primers SS2326)5034S5R3 and TD225)Mod-666-sal to amplify across the genomic DNA-transgene rDNA boundary in DSX-tTAV-Red-S with an expected amplicon size of 221 bp is shown in FIG. 8.

### 1.2 Detection of Wild-Type Alleles

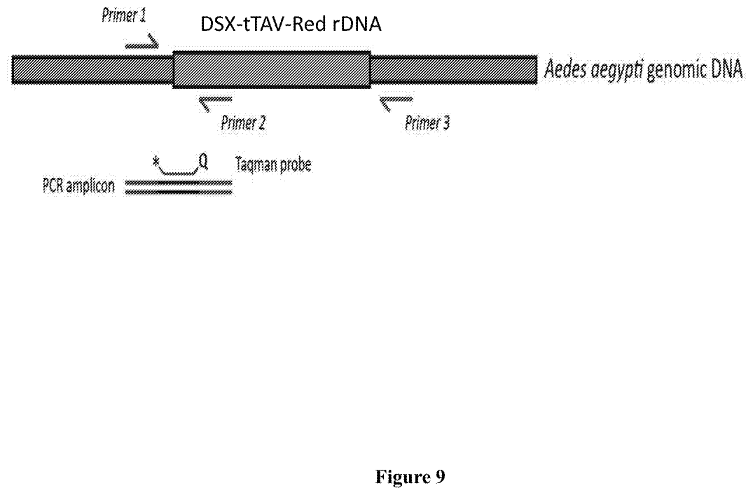

This assay method may also be adapted to detect the absence of the DSX-tTAV-Red rDNA (for example in DSX-tTAV-Red hemizygous individuals or in wild/wild-type mosquitoes) by designing a PCR reaction using primers annealing either side of the insertion site of the DSX-tTAV-Red rDNA (i.e., Primer 1 and Primer 3, FIG. 9). However, the presence of natural variation in the Aedes aegypti genome means that multiple primer sets may be required to amplify the various wild-type alleles that exist within a population (e.g., 3 wild-type alleles have been discovered in the lab population), and the discovery of further wild-type alleles will necessitate the design of further primer sets along the lines outlined here.

DSX-tTAV-Red-O wild-type alleles:

| | Primer ID | Sequence |
|---|---|---|
| Wild-type | | |
| allele 1 | | |
| Primer 1 | TD4037)5034Ofla2 | GATGGTCCCTAGAAACAGCTTTCC |
| | | (SEQ ID NO: 58) |
| Primer 3 | TD4039)5034Ofla4 | CTGTTGCTGCGCACGAAACAC |
| | | (SEQ ID NO: 59) |
| Wild-type | | |
| allele 2 | | |
| Primer 1 | TD4305)OX5034OWtF2 | TCGATCAACTAACTGAAATCGATGA (SEQ |
| | | ID NO: 60) |
| Primer 3 | TD4324)OX5034Wt2R | CCTAAGACCGTTAACATTTCAAGTGAC |
| | | (SEQ ID NO: 61) |
| Wild-type | | |
| allele 3 | | |
| Primer 1 | TD4393)OX5034Oal2F | CTTCGAGAGTAAGCGGAAACTCC |
| | | (SEQ ID NO: 62) |
| Primer 3 | TD4394)OX5034Oal2R | AGTATTAGCATCCGAAGCTCATGAC |
| | | (SEQ ID NO: 63) |

DSX-tTAV-S wild-type alleles:

| | Primer ID | Sequence |
|---|---|---|
| Wild-type | | |
| allele 1 | | |
| Primer 1 | SS2326)5034S5R1 | GCTTCATTAAGCAGAAACACTGA |
| | | (SEQ ID NO: 64) |
| Primer 3 | and SS2336)5034S3F2 | CATCTAACTCTACTTTGTGTGGGAATCA |
| | | (SEQ ID NO: 54) |

The PCR reaction mix for endpoint PCR detection methods is as follows:
12.8 μL MilliQ water
4 μL Biotaq buffer
0.5 μL 10× BSA
0.25 μL Primer 1 (10 μM)
0.25 μL Primer 2/Primer 3 (10 μM)
0.2 μL PCRBIO Taq polymerase (PCR Biosystems)
2 μl template gDNA at a concentration of approximately 10 ng/uL

### PCR Cycling:

Step 1) 94° C. 2 min
Step 2) 94° C. 15 s
Step 3) 60° C. 30 s -0.5° C./cycle
Step 4) 72° C. 15 s
Step 5) Repeat Steps 2 to 4 nine more times.
Step 6) 94° C. 15 s
Step 7) 55° C. 30 s
Step 8) 72° C. 15 s
Step 9) Repeat Steps 6 to 8 nineteen more times
Step 10) 72° C. 7 min
Step 11) 4° C. hold

PCR reaction mixtures are analysed on an appropriate agarose gel containing ethidium bromide or a similar DNA intercalating dye such as SYBR-safe.

2. SYBR-Green qPCR Detection Methods

In a variation on the endpoint PCR method, amplification may be detected by quantitative real-time PCR (qPCR) using an intercalating due such as SYBR-Green.

For qPCR amplification/detection, primers are designed to amplify a product of around 200 bp, using standard parameters:
Length 18-40 by
Predicted Tₘ approx 55° C.,
GC content between 30-70%
No significant secondary structure: dimer/hairpin dG <3.

Primers for DSX-tTAV-Red-O rDNA:

| | | | Amplicon size |
|---|---|---|---|
| | Primer ID | Sequence | (bp) |
| Primer 1 | TD4039)5034Ofla4 | CTGTTGCTGCGCACGAAACAC | 219 |
| | | (SEQ ID NO: 56) | |
| Primer 2 | SS218)PB3 | CAGACCGATAAAACACATGCGTCA | |
| | | (SEQ ID NO: 65) | |

Primers for DSX-tTAV-Red-S rDNA:

| | | | Amplicon size |
|---|---|---|---|
| | Primer ID | Sequence | (bp) |
| Primer 1 | TD225)Mod-666- | TGACAAGCACGCCTCACGGGAG | 218 |
| | sal | (SEQ ID NO: 55) | |
| Primer 2 | SS2326)5034S5R1 | GCTTCATTAAGCAGAAACACTGA | |
| | | (SEQ ID NO: 64) | |

Primers for DSX-tTAV-Red-O wild-type alleles:

| | | | Amplicon size |
|---|---|---|---|
| | Primer ID | Sequence | (bp) |
| Wild- | | | |
| type | | | |
| allele 1 | | | |
| Primer 1 | SS2399)OX5034OWT1R | TCGACTCATGGAGGTTTCACTG | 177 |
| | | (SEQ ID NO: 66) | |
| Primer 3 | SS2398)OX5034WT1F | ATGCGTTGCATTGTTATTCAATG | |
| | | (SEQ ID NO: 67) | |
| Wild- | | | |
| type | | | |
| allele 2 | | | |
| Primer 1 | TD4324)OX5034Wt2R | CCTAAGACCGTTAACATTTCAAGTGA | 333 |
| | | C | |
| | | (SEQ ID NO: 61) | |
| Primer 3 | SS2401)OX5034WT2F2 | AAATATCAGCCTCAAATAAGCACTT | |
| | | (SEQ ID NO: 68) | |
| Wild- | | | |
| type | | | |
| allele 3 | | | |
| Primer 1 | TD4394)OX5034Oal2R | AGTATTAGCATCCGAAGCTCATGAC | 330 |
| | | (SEQ ID NO: 63) | |
| Primer 3 | TD4393)OX5034Oal2F | CTTCGAGAGTAAGCGGAAACTCC | |
| | | (SEQ ID NO: 62) | |

Primers for DSX-tTAV-Red-S wild-type allele:

| | | | Amplicon size |
|---|---|---|---|
| | Primer ID | Sequence | (bp) |
| Wild- | | | |
| type | | | |
| allele 1 | | | |
| Primer 1 | 2335)5034S3F1 | CTATAGCTTTCTGGTGTACGGAATA | 218 |
| | | GAG | |
| | | (SEQ ID NO: 69) | |
| Primer 3 | SS2327)5034S5R2 | GGTCTCATAAGTATAACTCTGCACA | |
| | | GAG (SEQ ID NO: 70) | |

Aedes aegypti endogenous control Primers and Probe (IAP1):

| | Primer ID | Sequence |
|---|---|---|
| Primer 1 | SS2320)AedesF2 | TGCAGTAGTGATGAAGATGAACCA (SEQ ID |
| | | NO: 71) |
| Primer 2 | SS2321)AedesR2 | CGAAAATGCCGTATTGTACTCA (SEQ ID |
| | | NO: 72) |

Reaction mix per well (using qPCR kit, SuperMix-UDG Platinum SYBR Green Cat#10633863 from Thermofisher):
4.1 μl MilliQ Water
0.4 μL Primer 1 (10 μM)
0.4 μL Primer 2 (10 μM)
0.1 μl ROX
10 μl SYBR Mastermix
5 μl template gDNA, approx. 1 ng/uL

### PCR Cycling:

Step 1) 50° C. 2 mins
Step 2) 95° C. 2 min
Step 3) 95° C. 15 s
Step 4) 60° C. 30 s
Step 5) Repeat Steps 3 to 4 thirty-nine more times.

Cycle threshold (Ct) values are converted to concentration using a standard calibration curve, normalised to an endogenous control PCR (e.g. Aedes aegypti IAP1 gene, Table 9)) and assessed against predetermined limits of detection and limits of quantification to determine whether the OX5034 rDNA or wild-type alleles are present in the insect sample.

3. Taqman qPCR Detection Methods

PCR primers may be designed such that one primer anneals within the DSX-tTAV-Red rDNA and the other primer anneals to Aedes aegypti DNA in the region flanking the insertion site of the DSX-tTAV-Red rDNA. These primers may be used to amplify a PCR amplicon which may be detected by quantitative real-time PCR (qPCR) using a Taqman probe designed to anneal to the PCR amplicon. Using this method, the presence and copy number of the transgene may be detected, such that it is possible to differentiate mosquitoes homozygous for the DSX-tTAV-Red rDNA from those hemizygous for the DSX-tTAV-Red rDNA and also from wild-type/wild mosquitoes.

Primers and probes are designed according to the following rules:

Probes: no G as 5′ nucleotide
More Cs than Gs
Tₘ 68-70° C.

Primers are designed to amplify a product of approx. 100 bp (less than 150 bp), using the following rules:
Length 18-40 bp
Tm 58-60° C. (or 5-10° C. lower than the Tm of the probe)
More A/Ts than G/Cs in the last 5 nucleotides (no GC clamp)
GC between 30-70%
No significant secondary structure: dimer/hairpin dG <3.

For example, DSX-tTAV-Red-0 rDNA may be detected using the following primers and Taqman probe:

| | Primer ID | Sequence |
|---|---|---|
| Primer 1 | 2323)5034OtaqR | AAATGAAATTGCAAGTCCACTTT (SEQ ID |
| | | NO: 77) |
| Primer 2 | 2322)PB5taqF | GCGTCAATTTTACGCAGACTATC (SEQ ID |
| | | NO: 78) |
| Probe | 2325)5034OtaqPr | FAM-ACACCCGGCACGGTAAAATGTCA- |
| | | BHQ1 (SEQ ID NO: 79) |

For example, DSX-tTAV-Red-S rDNA may be detected using the following primers and Taqman probe:

| | Primer ID | Sequence |
|---|---|---|
| Primer 1 | 2408)5034StaqR | AGCAGAAACACTGAATTTTCAAAG (SEQ ID |
| | | NO: 80) |
| Primer 2 | 2322)PB5taqF | GCGTCAATTTTACGCAGACTATC (SEQ ID |
| | | NO: 81) |
| Probe | 2407)5034Staqpr | FAM- |
| | | ATGATGCGGAAGCGTAATCTTTACCCA- |
| | | BHQ1 (SEQ ID NO: 82) |

Thus this reaction may be carried out as a multiplexed Taqman qPCR reaction.

The DSX-tTAV-Red rDNA may be detected by using a FAM-labelled rDNA probe, while a HEX-labelled endogenous control probe may be used to detect an endogenous Aedes aegypti single copy gene PCR amplicon, e.g., IAP1 (inhibitor of apoptosis gene 1). Relative copy number may be calculated by first normalising DSX-tTAV-Red rDNA Ct (cycle threshold) values to endogenous control Ct values, and then to DSX-tTAV-Red rDNA Ct values obtained for a known OX5034 rDNA homozygous individual.

Primers for Aedes aegypti endogenous control primers and probe (IAP1):

| | Primer ID | Sequence |
|---|---|---|
| Primer 1 | SS2320)AedesF2 | TGCAGTAGTGATGAAGATGAACCA |
| | | (SEQ ID NO: 83) |
| Primer 2 | SS2321)AedesR2 | CGAAAATGCCGTATTGTACTCA (SEQ ID |
| | | NO: 84) |
| Probe | 551884)AedesPro | HEX- |
| | | AGACACCAGTCGGACTTGCAAAATCTG- |
| | | BHQ1 (SEQ ID NO: 85) |

Reaction mix per well (using Applied Biosystems Taqman Gene expression master mix, cat #4369016 from Thermofisher):
1.4 μl MilliQ H₂O
0.6 μl OX5034 rDNA Primer 1 (10 μM)
0.6 μl OX5034 rDNA Primer 2 (10 μM)
0.6 μl endogenous control Primer 1 (10 μM)
0.6 μl endogenous control Primer 2 (10 μM)
10 μl Gene expression Master mix
0.6 μl OX5034 rDNA probe (10 μM)
0.6 μl endogenous control probe (10 μM)
5 μl Template gDNA (approx. 1 ng/uL)

### PCR Cycling:

Step 1) 95° C. for 10 mins
Step 2)94° C. for 15 secs
Step 3) 60° C. for 1 min (read fluorescence at the end of this step)
Step 4) Repeat steps 2 and 3 thirty-nine more times.

### EMBODIMENTS

Embodiment 1. A doublesex (dsx) splice control module polynucleotide comprising, from 5′ to 3′: i. an exon 4 of dsx; ii. a truncated intron 4 of dsx comprising a 5′ terminal fragment of the dsx intron 4 and a 3′ fragment of the dsx intron 4; iii. an exon 5a of dsx; iv. an intron 5 of dsx; v. a modified exon 5b of dsx; vi. a truncated intron 6 of dsx comprising a 5′ terminal fragment of the dsx intron 6 and a 3′ fragment of the dsx intron 6; and vii. a 5′ fragment of exon 6.

Embodiment 2. The dsx splice control module of Embodiment 1 wherein said dsx is derived from Aedes aegypti (Aeadsx).

Embodiment 3. The dsx splice control module of Embodiment 1 or 2 wherein said modified exon 5b is modified to create an open reading frame for the entire exon.

Embodiment 4. The dsx splice control module of Embodiment 3 wherein said modified exon 5b comprises at least one substitution, insertion, and/or deletion to form an open reading frame for the entire exon.

Embodiment 5. The dsx splice control module of any of Embodiments 1 to 4 wherein said splice control module is spliced on a sex-specific basis when introduced into an insect.

Embodiment 6. The dsx splice control module of Embodiment 5 the insect is of the Order selected from the group consisting of Diptera, Phthiraptera, Hemiptera, Hymenoptera, Strepsiptera, Lepidoptera, and Coleoptera.

Embodiment 7. The dsx splice control module of Embodiment 6 wherein said insect is a Diptera of a species selected from the group consisting of Ceratitis capitata, Anastrepha ludens, Bactrocera dorsalis, Bactrocera oleae, Bactrocera cucurbitae, Ceratitis rosa, Rhagoletis cerasi, Bactrocera tyroni, Bactrocera zonata, Anastrepha suspense, and Anastrepha obliqua.

Embodiment 8. The dsx splice control module of Embodiment 6 wherein said Diptera is a mosquito of a genus selected from the group consisting of Stegomyia, Aedes, Anopheles and Culex.

Embodiment 9. The dsx splice control module of Embodiment 8 wherein said mosquito is a species selected from the group consisting of Aedes aegypti, Aedes albopictus, Anopheles stephensi, Anopheles albimanus and Anopheles gambiae.

Embodiment 10. The dsx splice control module of Embodiment 6 wherein said insect is a Calliphoridae species selected from the group consisting of Cochliomyia hominivorax, Chrysomya bezziana, and Lucilia cuprina).

Embodiment 11. The dsx splice control module of Embodiment 6 wherein said Lepidoptera insect is a species selected from the group consisting of Cydia pomonella, Bombyx mori, Pectinophora gossypiella, Plutella xylostella, Lymantria dispar, Amyelois transitella, Anarsia lineatella, Tryporyza incertulas, and Heliothinae spp.

Embodiment 12. The dsx splice control module of Embodiment 6 wherein said Coleoptera insect is a species selected from the group consisting of Popilla japonica, Graphognatus spp., Anthonomous grandis, Diabrotica spp. and Leptinotarsa decemlineata.

Embodiment 13. A gene expression system comprising a polynucleotide comprising a doublesex (dsx) splice control module of any of Embodiments 1 to 5 operably linked to a polynucleotide encoding a heterologous protein.

Embodiment 14. The gene expression system of Embodiment 13 wherein said dsx splice control module is derived from Aedes aegypti (Aeadsx).

Embodiment 15. The gene expression system of Embodiment 13 or 14 wherein said heterologous protein is a protein that is lethal, deleterious or sterilizing effect to an insect.

Embodiment 16. The gene expression system of Embodiment 15 wherein said protein that is lethal or deleterious to an insect is a protein selected from the group consisting of a synthetic tetracycline repressive transcriptional activator protein (tTAV), an apoptosis-inducing factor, Hid, Reaper (Rpr), and Nipp1Dm.

Embodiment 17. The gene expression system of Embodiment 16 wherein said tTAV is selected from the group consisting of tTAV, tTAV2 or tTAV3.

Embodiment 18. The gene expression system of Embodiment 13 wherein said polynucleotide encoding said heterologous protein further comprises a polynucleotide sequence encoding a Fusion Leader Polypeptide fused in frame to the 5′ and of said polynucleotide encoding said tTAV.

Embodiment 19. The gene expression system of Embodiment 18 wherein said Fusion Leader Polypeptide is ubitquitin.

Embodiment 20. The gene expression system of any of Embodiments 13-19 further comprising a 5′ untranslated region (5′UTR) operably linked 5′ of said splice control module.

Embodiment 21. The gene expression system of Embodiment 20 wherein said 5′UTR comprises a promoter operable in an insect.

Embodiment 22. The gene expression system of Embodiment 21 wherein said promoter is a Drosophila melanogaster minimal HSP70 promoter (DmHsp70).

Embodiment 23. The gene expression system of Embodiment 21 or 22 wherein said 5′UTR further comprises a tetracycline responsive operator.

Embodiment 24. The gene expression system of any of Embodiments to 13 to 23 further comprising a 3′ untranslated region (3′UTR) operably linked 3′ of said tTAV.

Embodiment 25. The gene expression system of Embodiment 24 wherein said 3′UTR is an SV40 3′UTR.

Embodiment 26. An expression vector plasmid comprising the gene expression system of any of Embodiments 13 to 25.

Embodiment 27. The expression vector plasmid of Embodiment 26 further comprising a polynucleotide encoding a fluorescent marker protein.

Embodiment 28. The expression vector plasmid of Embodiment 27 wherein said fluorescent marker protein is DsRed2.

Embodiment 29. The expression vector plasmid of Embodiment 27 wherein said polynucleotide encoding said fluorescent marker protein is operably linked to a promoter.

Embodiment 30. The expression vector plasmid of Embodiment 29 wherein said promoter is an IE1 promoter.

Embodiment 31. The expression vector plasmid of any of Embodiments 26 to 30 further comprising piggyBac transposable element ends to direct incorporation of said expression vector plasmid into the chromosome of an insect.

Embodiment 32. A genetically engineered insect comprising a gene expression system incorporated into a chromosome of said insect, said gene expression system comprising a polynucleotide construct comprising: i. a doublesex (dsx) splice control module wherein said splice control module comprises the components from 5′ to 3′: i. an exon 4; ii. a truncated intron 4 of dsx comprising a 5′ terminal fragment of the dsx intron 4 and a 3′ fragment of the dsx intron 4; iii. an exon 5a; iv. an intron 5 of dsx; v. a modified exon 5b of said dsx; vi. a truncated intron 6 of dsx comprising a 5′ terminal fragment of the dsx intron 6 and a 3′ fragment of the dsx intron 6; and vii. a 5′ fragment of exon 6; ii. a polynucleotide encoding ubiquitin fused in frame to the 5′ and of a polynucleotide encoding tTAV positioned 3′ of said splice control module; iii. a tetO element; and iv. a 5′UTR positioned 5′ of said splice control module wherein said 5′UTR comprises a promoter.

Embodiment 33. The genetically engineered insect of Embodiment 32 wherein said insect is a mosquito.

Embodiment 34. The genetically engineered insect of Embodiment 33 wherein said mosquito is a mosquito of the genus Aedes, Anopheles, or Culex.

Embodiment 35. The genetically engineered insect of Embodiment 34 wherein said mosquito is Aedes aegypti.

Embodiment 36. The genetically engineered insect of Embodiment 32 wherein said gene expression system further comprises a polynucleotide encoding a fluorescent protein.

Embodiment 37. The genetically engineered insect of Embodiment 36 wherein said fluorescent protein is DsRed2.

Embodiment 38. The genetically engineered insect of Embodiment 36 wherein said fluorescent protein is operably linked to a promoter.

Embodiment 39. The genetically engineered insect of Embodiment 38 wherein said fluorescent protein is DsRed2 and said polynucleotide encoding said fluorescent protein is operably linked to an IE1 promoter.

Embodiment 40. A method of producing genetically engineered insects comprising modifying an insect's chromosome by inserting a gene expression system, wherein said gene expression system comprises: i. a doublesex (dsx) splice control module wherein said splice control module comprises the components from 5′ to 3′: i. an exon 4; ii. a truncated intron 4 of dsx comprising a 5′ terminal fragment of the dsx intron 4 and a 3′ fragment of the dsx intron 4; iii. an exon 5a; iv. an intron 5 of dsx; v. a modified exon 5b of said dsx; vi. a truncated intron 6 of dsx comprising a 5′ terminal fragment of the dsx intron 6 and a 3′ fragment of the dsx intron 6; and vii. a 5′ fragment of exon 6; ii. a polynucleotide encoding ubiquitin fused in frame to the 5′ and of a polynucleotide encoding tTAV positioned 3′ of said splice control module; iii. a tetO element; and iv. a 5′UTR positioned 5′ of said splice control module wherein said 5′UTR comprises a promoter.

Embodiment 41. The method of Embodiment 40 wherein said insect is a mosquito.

Embodiment 42. The method of Embodiment 41 wherein said mosquito is a mosquito of the genus Aedes, Anopheles, or Culex.

Embodiment 43. The method of Embodiment 42 wherein said mosquito is Aedes aegypti.

Embodiment 44. The method of Embodiment 40 wherein said gene expression system further comprises a polynucleotide encoding a fluorescent protein.

Embodiment 45. The method of Embodiment 44 wherein said fluorescent protein is DsRed2.

Embodiment 46. The method of Embodiment 44 wherein said fluorescent protein is operably linked to a promoter.

Embodiment 47. The method of Embodiment 46 wherein said fluorescent protein is DsRed2 and said polynucleotide encoding said fluorescent protein is operably linked to an IE1 promoter.

Embodiment 48. A method of selectively rearing male genetically engineered insects comprising, rearing a genetically engineered insect of any of Embodiments 32 to 39 wherein said rearing is in the absence of tetracycline.

Embodiment 49. A male genetically engineered male insect produced by the method of Embodiment 48.

Embodiment 50. A method of reducing a wild insect population comprising contacting said wild insect population with a plurality of the male genetically engineered insects of Embodiment 49 wherein said male genetically engineered insects mate with wild female insects of the same species.

Embodiment 51. The method of Embodiment 50 wherein said insect is a mosquito.

Embodiment 52. The method of Embodiment 51 wherein said mosquito is a mosquito of the genus Aedes, Anopheles, or Culex.

Embodiment 53. The method of Embodiment 52 wherein said mosquito is Aedes aegypti.

Embodiment 54. An Aedes aegypti chromosomal target site located on Aedes aegypti supercontig 1.420 position 324552, between gene AAEL009696 and an exon in gene AAEL009706 wherein the target site comprises a heterologous nucleic acid.

Embodiment 55. The Aedes aegypti chromosomal target site of Embodiment 54 wherein the target site is located between nucleotides 1845 and 1850 of SEQ ID NO: 46.

Embodiment 56. The Aedes aegypti chromosomal target site of Embodiment 54 wherein the target site is flanked 5′ by nucleotides 1443 to 1845 of SEQ ID NO: 46 and flanked 3′ by nucleotides 1859 to 2222 of SEQ ID NO: 46.

Embodiment 57. A method of making a transgenic Aedes aegypti mosquito comprising inserting a heterologous nucleic acid at a position on Aedes aegypti supercont1.420 position 324552, between gene AAEL009696 and an exon in gene AAEL009706.

Embodiment 58. The method of Embodiment 57 wherein the heterologous nucleic acid is inserted on Aedes aegypti between nucleotides 1845 and 1850 of SEQ ID NO: 46 or between SEQ ID NO: 73 and SEQ ID NO: 74.

Embodiment 59. The method of Embodiment 57 wherein the inserted heterologous nucleic acid is flanked 5′ by nucleotides 1443 to 1845 of SEQ ID NO: 46 and flanked 3′ by nucleotides 1859 to 2222 of SEQ ID NO:46.

Embodiment 60. An Aedes aegypti chromosomal target site located on Aedes aegypti supercontig 1.19 position 2799615.

Embodiment 61. The Aedes aegypti chromosomal target site of Embodiment 60 wherein the target site is flanked 5′ by nucleotides of SEQ ID NO: 75 and flanked 3′ by nucleotides of SEQ ID NO: 76.

Embodiment 62. A method of making a transgenic Aedes aegypti mosquito comprising inserting a heterologous nucleic acid at a position on Aedes aegypti supercontig 1.19 position 2799615.

Embodiment 63. The method of Embodiment 62 wherein the heterologous nucleic acid is inserted on Aedes aegypti gDNA between a nucleotide comprising SEQ ID NO: 75 and a nucleotide comprising SEQ ID NO: 76.

Embodiment 64. The method of Embodiment 62 wherein the inserted heterologous nucleic acid is flanked 5′ by nucleotides of SEQ ID NO:75 and flanked 3′ by nucleotides of SEQ ID NO:76. Embodiment 65. A genetically engineered Aedes aegypti mosquito comprising a heterologous nucleic acid inserted at a position on Aedes supercont 1.420 position 324552, between gene AAEL009696 and an exon in gene AAEL009706 wherein said heterologous nucleic acid comprises gene expression system comprising a splice control module operatively linked to a polynucleotide encoding a Fusion Leader Polypeptide fused in frame to the 5′ of a polynucleotide encoding a gene of interest to be differentially expressed, and wherein said polynucleotide encoding said Fusion Leader Polypeptide is positioned 3′ of said splice control module; a tetO element, and a 5′UTR positioned 5′ of said splice control module wherein said 5′UTR comprises a promotor.

Embodiment 66. A genetically engineered Aedes aegypti mosquito comprising a heterologous nucleic acid inserted at a position on Aedes aegypti supercontig 1.19 position 2799615 wherein said heterologous nucleic acid comprises gene expression system comprising a splice control module operatively linked to a polynucleotide encoding a Fusion Leader Polypeptide fused in frame to the 5′ of a polynucleotide encoding a gene of interest to be differentially expressed, and wherein said polynucleotide encoding said Fusion Leader Polypeptide is positioned 3′ of said splice control module; a tetO element; and a 5′UTR positioned 5′ of said splice control module wherein said 5′UTR comprises a promoter.

Embodiment 67. A genetically engineered Aedes aegypti mosquito of Embodiment 65 or 66 wherein said splice control module is derived from a gene selected from the group consisting of tra, dsx, and Actin-4.

Embodiment 68. A method of detecting the presence of a DNA molecule selected from the group consisting of SEQ ID NO: 36 and SEQ ID NO: 37 in a DNA sample, the method comprising: (a) extracting a DNA sample from an insect; (b) contacting the DNA sample with a DNA molecule that can act as a primer identified within 3 kb of the insertion site upstream or downstream of the DSX-tTAV-Red insertions sites, wherein said DNA molecule is a DNA sequence that hybridizes under stringent hybridization conditions with the DNA molecule selected from the group consisting of the sequences adjacent to the DSX-tTAV-Red inserts and does not hybridize under the stringent hybridization conditions with a DNA sample not containing the DNA molecule identified as the sequences adjacent to the DSX-tTAV-Red insertion site; (c) using a second primer that is DNA molecule that can act as a primer identified within an rDNA comprising the sequence of SEQ ID NO:36 or an rDNA comprising the sequence of SEQ ID NO:37, wherein said DNA molecule is a DNA sequence that hybridizes under stringent hybridization conditions with the DNA molecule selected from the group consisting of the rDNA comprising the sequence of SEQ ID NO:36 or the rDNA comprising the sequence of SEQ ID NO:37 insert and does not hybridize under the stringent hybridization conditions with a DNA sample not containing the DNA molecule comprising the sequence of SEQ ID NO:36 or SEQ ID NO:37; i. where the hybridization of DNA sequences capable of acting as primers allows the specific amplification of DNA from DSX-tTAV-Red strains such that the product is specific for the DSX-tTAV-Red-O or the DSX-tTAV-Red-S strain through DNA amplification methods and detection of said amplified DNA, ii. or where a DNA probe specific for strain DSX-tTAV-Red-O and DSX-tTAV-Red -S may allow the specific detection of DSX-tTAV-Red-O and DSX-tTAV-Red-S strains through hybridization without further DNA amplification; (d) subjecting the sample and probe to stringent hybridization conditions; and detecting hybridization of the probe to the DNA through DNA amplification using two primers or through other techniques relying on hybridization of sequences specific for DSX-tTAV-Red-S and DSX-tTAV-Red-O.

Embodiment 69. A DNA molecule comprising any of the primers provided in the Specification.

Embodiment 70. A DNA detection kit comprising: at least one DNA molecule of sufficient length of contiguous nucleotides homologous or complementary to SEQ ID NO: 36 or SEQ ID NO: 37 that functions as a DNA primer or probe specific for DSX-tTAV-Red-O and DSX-tTAV-Red-S and their progeny.

1. A genetically engineered insect comprising a gene expression system incorporated into a chromosome of said insect, said gene expression system comprising:
(a) a doublesex (dsx) splice control module wherein said splice control module comprises the components from 5′ to 3′:
i. an exon 4 of dsx;
ii. a truncated intron 4 of dsx comprising a 5′ terminal fragment of the dsx intron 4 and a 3′ fragment of the dsx intron 4;
iii. an exon 5a of dsx;
iv. an intron 5 of dsx;
v. a modified exon 5b of dsx;
vi. a truncated intron 6 of dsx comprising a 5′ terminal fragment of the dsx intron 6 and a 3′ fragment of the dsx intron 6; and
vii. a 5′ fragment of exon 6 of dsx;
(b) a polynucleotide sequence encoding a heterologous protein, wherein said polynucleotide sequence is positioned 3′ of said splice control module;
(c) a tetO element; and
(d) a 5′UTR positioned 5′ of said splice control module, wherein said 5′UTR comprises a promoter.

2. The genetically engineered insect of claim 1, wherein said modified exon 5b is modified with at least one substitution, insertion, and/or deletion to create an open reading frame for the entire exon, and wherein said splice control module is spliced on a sex-specific basis in said insect.

3. The genetically engineered insect of claim 1, wherein said insect is of the Order selected from the group consisting of Diptera, Phthiraptera, Hemiptera, Hymenoptera, and Strepsiptera.

4. The genetically engineered insect of claim 1, wherein said insect is of a species selected from the group consisting of Ceratitis capitata, Anastrepha ludens, Bactrocera dorsalis, Bactrocera oleae, Bactrocera cucurbitae, Ceratitis rosa, Rhagoletis cerasi, Bactrocera tyroni, Bactrocera zonata, Anastrepha suspense, and Anastrepha obliqua.

5. The genetically engineered insect of claim 1, wherein said insect is a mosquito of the genus Stegomyia, Aedes, Anopheles, or Culex.

6. The genetically engineered insect of claim 1, wherein said insect is of a species selected from the group consisting of Aedes aegypti, Aedes albopictus, Anopheles stephensi, Anopheles albimanus, and Anopheles gambiae.

7. The genetically engineered insect of claim 1, wherein said insect is Aedes aegypti.

8. The genetically engineered insect of claim 1, wherein said heterologous protein is lethal, deleterious, or sterilizing to the insect.

9. The genetically engineered insect of claim 1, wherein said heterologous protein is a protein selected from the group consisting tTAV, tTAV2, tTAV3, an apoptosis-inducing factor, Hid, Reaper (Rpr), and Nipp1Dm.

10. The genetically engineered insect of claim 1, wherein said gene expression system further comprises a polynucleotide sequence encoding a ubiquitin Fusion Leader Polypeptide fused in frame to the 5′ end of said polynucleotide sequence encoding said heterologous protein.

11. The genetically engineered insect of claim 1, wherein said gene expression system comprises a polynucleotide encoding a ubiquitin fused in frame to the 5′ end of a polynucleotide encoding tTAV.

12. The genetically engineered insect of claim 1, wherein said promoter is a Drosophila melanogaster minimal HSP70 promoter (DmHsp70).

13. The genetically engineered insect of claim 1, wherein said gene expression system further comprises a 3′ untranslated region (3′UTR) operably linked 3′ of said polynucleotide sequence encoding said heterologous protein.

14. The genetically engineered insect of claim 13, wherein said 3′UTR is an SV40 3′UTR.

15. The genetically engineered insect of claim 1, wherein said gene expression system further comprises a polynucleotide encoding a fluorescent protein.

16. The genetically engineered insect of claim 1, wherein said insect is a male.

17. The genetically engineered insect of claim 1, wherein said insect is an Aedes aegypti mosquito, and wherein said gene expression system is inserted at a position on Aedes aegypti supercontig 1.420 position 324552, between gene AAEL009696 and an exon in gene AAEL009706.

18. The genetically engineered insect of claim 1, wherein said insect is an Aedes aegypti mosquito, and wherein said gene expression system is inserted at a position on Aedes aegypti supercontig 1.19 position 2799615.

19. A method of producing genetically engineered insects comprising modifying an insect's chromosome by inserting a gene expression system, wherein said gene expression system comprises:
(a) a doublesex (dsx) splice control module wherein said splice control module comprises the components from 5′ to 3′:
i. an exon 4 of dsx;
ii. a truncated intron 4 of dsx comprising a 5′ terminal fragment of the dsx intron 4 and a 3′ fragment of the dsx intron 4;
iii. an exon 5a of dsx;
iv. an intron 5 of dsx;
v. a modified exon 5b of dsx;
vi. a truncated intron 6 of dsx comprising a 5′ terminal fragment of the dsx intron 6 and a 3′ fragment of the dsx intron 6; and
vii. a 5′ fragment of exon 6 of dsx;
(b) a polynucleotide encoding ubiquitin fused in frame to the 5′ end of a polynucleotide encoding tTAV positioned 3′ of said splice control module;
(c) a tetO element; and
(d) a 5′UTR positioned 5′ of said splice control module, wherein said 5′UTR comprises a promoter.

20. A method of reducing a wild insect population comprising contacting said wild insect population with a plurality of the male genetically engineered insects of claim 16, wherein said male genetically engineered insects are mosquitos of the genus Aedes, Anopheles, or Culex, and wherein said insects mate with wild female insects of the same species.
